(19) United States
(12) Patent Application Publication
(10) Pub. No.: US 2024/0197871 A1
(43) Pub. Date: Jun. 20, 2024

(54) COMBINATION OF A CAR T CELL THERAPY AND AN IMMUNOMODULATORY COMPOUND FOR TREATMENT OF LYMPHOMA

(71) Applicant: Juno Therapeutics, Inc., Seattle, WA (US)

(72) Inventors: Nikolaus Sebastian TREDE, Seattle, WA (US); Tim PULHAM, Boudry (CH); Fan WU, Summit, NJ (US); Michael POURDEHNAD, San Francisco, CA (US); Joseph Andrew DESTEFANO, Summit, NJ (US); Maria Soraya CARRANCIO ANTON, San Diego, CA (US); Tonia Jane BUCHHOLZ, San Francisco, CA (US); Michael PORTS, Seattle, WA (US); Jim QIN, Seattle, WA (US); Ronald DUBOWY, Seattle, WA (US); Nicolas BRAND, Boudry (CH)

(73) Assignee: Juno Therapeutics, Inc., Seattle, WA (US)

(21) Appl. No.: 18/284,800

(22) PCT Filed: Mar. 29, 2022
(86) PCT No.: PCT/US2022/022358

§ 371 Date: Sep. 28, 2023

Related U.S. Application Data
(60) Provisional application No. 63/277,134, filed on Nov. 8, 2021.
(60) Provisional application No. 63/167,599, filed on Mar. 29, 2021.

(51) Int. Cl. A61K 39/00; A61K 31/5377; A61K 31/675; A61K 31/7076; A61P 35/00; C07K 14/705; C07K 14/725; C07K 16/28

(52) U.S. Cl. CPC A61K 39/4611; A61K 31/5377; A61K 31/675; A61K 31/7076; A61K 39/4631; A61K 39/464412; A61P 35/00; C07K 14/7051; C07K 14/70521; C07K 14/70578; C07K 16/2803; A61K 2239/26; A61K 2239/38; A61K 2239/39; C07K 2319/02; C07K 2319/03

(57) ABSTRACT

Provided are methods, compositions, uses and articles of manufacture of combination therapies involving immunotherapies, such as adoptive cell therapy, e.g., T cell therapy, and the use of (S)-2-(2,6-dioxopiperidin-3-yl)-4-((2-fluoro-4-((3-morpholinoazetidin-1-yl)methyl)benzyl)amino)isoindoline-1,3-dione, or an enantiomer or mixture of enantiomers thereof, or a pharmaceutically acceptable salt, solvate, hydrate, co-crystal, clathrate, or polymorph thereof, for treating subjects with disease and conditions such as certain B cell malignancies, and related methods, compositions, uses and articles of manufacture. The cells generally express recombinant receptors such as chimeric antigen receptors (CARs). In some embodiments, the disease or condition is a non-Hodgkin lymphoma (NHL), such as relapsed or refractory NHL or specific NHL subtype.

### CROSS-REFERENCE TO RELATED APPLICATIONS

This application claims priority to U.S. Provisional Application No. 63/167,599, filed Mar. 29, 2021, and U.S. Provisional Application No. 63/277,134, filed Nov. 8, 2021, the contents of each of which are hereby incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 735042024540SeqList.txt, created Mar. 27, 2022, which is 36 kilobytes in size. The information in the electronic format of the Sequence Listing is incorporated by reference in its entirety.

### FIELD

The present disclosure relates in some aspects to methods, compositions, uses, and articles of manufacture of combination therapies involving immunotherapies, such as adoptive cell therapy, e.g., T cell therapy, and the use of (S)-2-(2,6-dioxopiperidin-3-yl)-4-((2-fluoro-4-((3-morpholinoazetidin-1-yl)methyl)benzyl)amino)isoindoline-1,3-dione, or an enantiomer or mixture of enantiomers thereof, or a pharmaceutically acceptable salt, solvate, hydrate, co-crystal, clathrate, or polymorph thereof, for treating subjects with disease and conditions such as certain B cell malignancies, and related methods, compositions, uses, and articles of manufacture. The T cell therapy includes cells that express recombinant receptors such as chimeric antigen receptors (CARs). In some embodiments, the disease or condition is a non-Hodgkin lymphoma (NHL), such as relapsed or refractory NHL or specific NHL subtype.

### BACKGROUND

Various strategies are available for immunotherapy, for example administering engineered T cells for adoptive therapy. For example, strategies are available for engineering T cells expressing genetically engineered antigen receptors, such as CARs, and administering compositions containing such cells to subjects. Improved strategies are needed to improve efficacy of the cells, for example, improving the persistence, activity, and/or proliferation of the cells upon administration to subjects. Provided are methods, compositions, kits, and systems that meet such needs.

### SUMMARY

Provided herein in some embodiments is a method of treating a cancer expressing CD19, the method comprising administering to a subject having a cancer expressing CD19 a combination therapy comprising: (i) a T cell therapy comprising a dose of engineered cells comprising T cells expressing a chimeric antigen receptor (CAR) directed against the cancer; and (ii) a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof.

In some of any of the provided methods, the cancer expressing CD19 is a lymphoma.

Also provided herein in some embodiments is a method of treating a lymphoma, the method comprising administering to a subject having a cancer that is a lymphoma a combination therapy comprising: (i) a T cell therapy comprising a dose of engineered cells comprising T cells expressing a chimeric antigen receptor (CAR) that binds cluster of differentiation 19 (CD19); and (ii) a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof.

In some of any of the provided methods, the administration of the compound is initiated prior to the administration of the T cell therapy.

Also provided herein in some embodiments is a method of treating a cancer expressing CD19, the method comprising administering to a subject having a cancer expressing CD19 a T cell therapy comprising a dose of engineered cells comprising T cells expressing a chimeric antigen receptor (CAR) that targets the cancer, wherein the T cell therapy is administered in a combination therapy with a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof, wherein the administration of the compound is initiated prior to the administration of the T cell therapy.

In some of any of the provided methods, the cancer expressing CD19 is a lymphoma.

Also provided herein in some embodiments is a method of treating a lymphoma, the method comprising administering to a subject having a cancer that is a lymphoma a T cell therapy comprising a dose of engineered cells comprising T cells expressing a chimeric antigen receptor (CAR) that binds cluster of differentiation 19 (CD19), wherein the T cell therapy is administered in a combination therapy with a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof, wherein the administration of the compound is initiated prior to the administration of the T cell therapy.

In some of any of the provided methods, the administration of the compound is initiated subsequently to the administration of the T cell therapy.

Also provided herein in some embodiments is method of treating a cancer expressing CD19, the method comprising administering to a subject having a cancer expressing CD19 a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof, wherein the compound is administered in a combination therapy with a T cell therapy comprising a dose of engineered cells comprising T cells expressing a chimeric antigen receptor (CAR) that targets the cancer, wherein the administration of the compound is initiated subsequently to the administration of the T cell therapy.

In some of any of the provided methods, the cancer expressing CD19 is a lymphoma.

Also provided herein in some embodiments is method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof, wherein the compound is administered in a combination therapy with a T cell therapy comprising a dose of engineered cells comprising T cells expressing a chimeric antigen receptor (CAR) that binds cluster of differentiation 19 (CD19), wherein the administration of the compound is initiated subsequently to the administration of the T cell therapy.

In some of any of the provided methods, the T cell therapy is administered on Day 1 of the combination therapy.

In some of any of the provided methods, the administration of the compound is initiated between Day 1 and Day 29, inclusive, of the combination therapy.

In some of any of the provided methods, the compound is administered as a plurality of doses, wherein each dose is between at or about 0.1 mg and at or about 0.6 mg, inclusive.

In some of any of the provided methods, the compound is administered as a plurality of intermittent doses administered no more than once weekly.

Also provided herein in some embodiments is a method of treating a lymphoma, the method comprising administering to a subject having a lymphoma a combination therapy comprising: (i) a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and (ii) a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

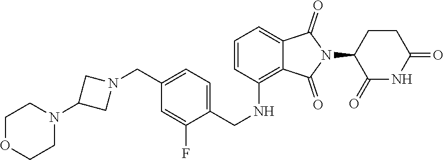 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein the compound is administered in an intermittent (i.e., non-daily) dosing regimen.

Also provided herein in some embodiments is a method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

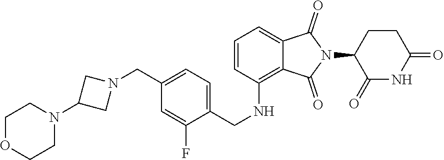 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy, and the compound is administered in an intermittent (i.e., non-daily) dosing regimen.

In some of any of the provided methods, the compound is administered as a plurality of intermittent doses. In some of any of the provided methods, the compound is administered no more than once weekly.

In some of any of the provided methods, each dose of the compound is between at or about 0.1 mg and at or about 0.6 mg, inclusive.

In some of any of the provided methods, the administration of the compound is initiated between Day 1 and Day 29, inclusive, of the combination therapy.

Also provided herein in some embodiments is a method of treating a lymphoma, the method comprising administering to a subject having a lymphoma a combination therapy comprising: (i) a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and (ii) a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

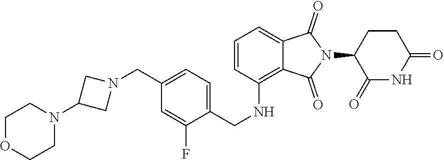 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein the compound is administered as a plurality of intermittent doses administered no more than once weekly, wherein each dose is between at or about 0.1 mg and at or about 0.6 mg, inclusive, wherein the administration of the compound is initiated between Day 1 and Day 29, inclusive, of the combination therapy.

Also provided herein in some embodiments is a method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

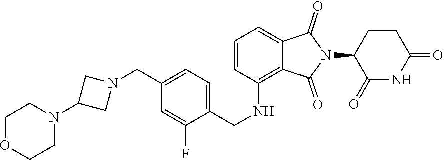 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein: the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and the compound is administered as a plurality of intermittent doses administered no more than once weekly, wherein each dose is between at or about 0.1 mg and at or about 0.6 mg, inclusive, and wherein the administration of the compound is initiated between Day 1 and Day 29, inclusive, of the combination therapy.

In some of any of the provided methods, the administration of the compound is initiated between Day 1 and Day 22, inclusive. In some of any of the provided methods, the administration of the compound is initiated between Day 1 and Day 15, inclusive. In some of any of the provided methods, the administration of the compound is initiated between Day 8 and Day 15, inclusive.

In some of any of the provided methods, the administration of the compound is initiated at or about Day 1. In some of any of the provided methods, the administration of the compound is initiated at or about Day 8. In some of any of the provided methods, the administration of the compound is initiated at or about Day 15.

In some of any of the provided methods, each dose of the plurality of intermittent doses is the same.

In some of any of the provided methods, the compound is administered once weekly. In some of any of the provided methods, the compound is administered once every 7 days (Q7D). In some of any of the provided methods, the compound is administered once every two weeks. In some of any of the provided methods, the compound is administered once every 14 days (Q14D).

In some of any of the provided methods, the compound is administered for at least 12 weeks after the administration of the T cell therapy. In some of any of the provided methods, the compound is administered for up to 12 weeks after the administration of the T cell therapy.

In some of any of the provided methods, the compound is administered on Days 1, 8, 15, 22, 29, 36, 43, 50, 57, 64, 71, 78, and 85. In some of any of the provided methods, the compound is administered on Days 8, 15, 22, 29, 36, 43, 50, 57, 64, 71, 78, and 85. In some of any of the provided methods, the compound is administered on Days 15, 22, 29, 36, 43, 50, 57, 64, 71, 78, and 85. In some of any of the provided methods, the compound is administered on Days 8, 22, 36, 50, 64, and 78.

In some of any of the provided methods, the dose of the compound is between at or about 0.3 mg and at or about 0.6 mg, inclusive. In some of any of the provided methods, the dose of the compound is at or about 0.6 mg.

In some of any of the provided methods, the dose of the compound is between at or about 0.2 mg and at or about 0.4 mg, inclusive. In some of any of the provided methods, the dose of the compound is at or about 0.4 mg. In some of any of the provided methods, the dose of the compound is less than 0.4 mg. In some of any of the provided methods, the dose of the compound is at or about 0.3 mg. In some of any of the provided methods, the dose of the compound is at or about 0.2 mg.

Also provided herein in some embodiments is a method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

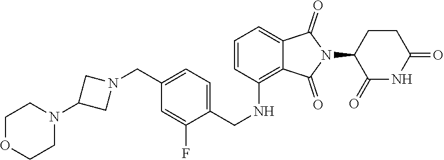 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein: the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and the compound is administered as a plurality of intermittent doses administered once every seven days (Q7D) and is administered on Days 8, 15, 22, 29, 36, 43, 50, 57, 64, 71, 78, and 85, wherein each dose is 0.3 mg.

Also provided herein in some embodiments is a method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

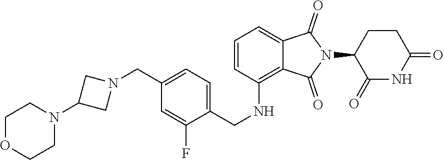 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein: the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and the compound is administered as a plurality of intermittent doses administered once every seven days (Q7D) and is administered on Days 1, 8, 15, 22, 29, 36, 43, 50, 57, 64, 71, 78, and 85, wherein each dose is 0.3 mg.

Also provided herein in some embodiments is a method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

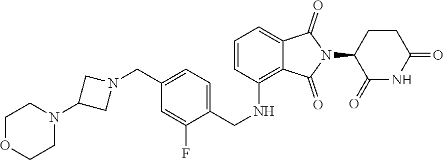 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein: the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and the compound is administered as a plurality of intermittent doses administered once every seven days (Q7D) and is administered on Days 15, 22, 29, 36, 43, 50, 57, 64, 71, 78, and 85, wherein each dose is 0.3 mg.

Also provided herein in some embodiments is a method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

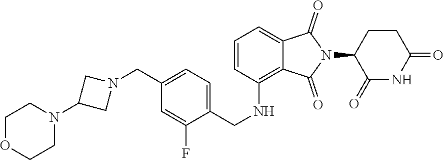 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein: the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and the compound is administered as a plurality of intermittent doses administered once every seven days (Q7D) and is administered on Days 8, 15, 22, 29, 36, 43, 50, 57, 64, 71, 78, and 85, wherein each dose is 0.4 mg.

Also provided herein in some embodiments is a method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

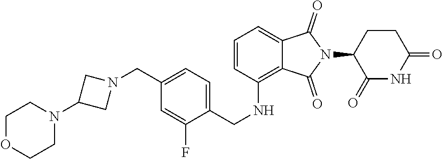 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein: the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and the compound is administered as a plurality of intermittent doses administered once every 14 days (Q14D) and is administered on Days 8, 22, 36, 50, 64, and 78, wherein each dose is 0.3 mg.

Also provided herein in some embodiments is a method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

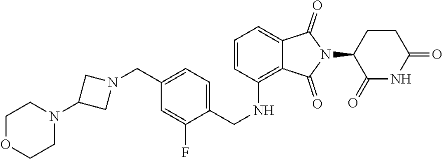 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein: the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and the compound is administered as a plurality of intermittent doses administered once every seven days (Q7D) and is administered on Days 8, 15, 22, 29, 36, 43, 50, 57, 64, 71, 78, and 85, wherein each dose is 0.2 mg.

Also provided herein in some embodiments is a method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

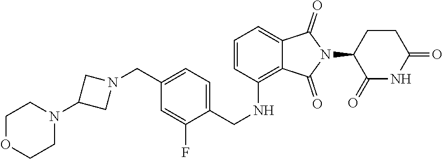 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein: the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and the compound is administered as a plurality of intermittent doses administered once every seven days (Q7D) and is administered on Days 8, 15, 22, 29, 36, 43, 50, 57, 64, 71, 78, and 85, wherein each dose is 0.6 mg.

In some of any of the provided methods, the compound is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof.

In some of any of the provided methods, the compound is or comprises a pharmaceutically acceptable salt of (S)-2-(2,6-dioxopiperidin-3-yl)-4-((2-fluoro-4-((3-morpholinoazetidin-1-yl)methyl)benzyl)amino)isoindoline-1,3-dione. In some of any of the provided methods, the compound is or comprises a hydrate of (S)-2-(2,6-dioxopiperidin-3-yl)-4-((2-fluoro-4-((3-morpholinoazetidin-1-yl)methyl)benzyl)amino)isoindoline-1,3-dione. In some of any of the provided methods, the compound is or comprises a solvate of (S)-2-(2,6-dioxopiperidin-3-yl)-4-((2-fluoro-4-((3-morpholinoazetidin-1-yl)methyl)benzyl)amino)isoindoline-1,3-dione. In some of any of the provided methods, the compound is or comprises (S)-2-(2,6-dioxopiperidin-3-yl)-4-((2-fluoro-4-((3-morpholinoazetidin-1-yl)methyl)benzyl)amino)isoindoline-1,3-dione.

In some of any of the provided methods, at the time of the initiation of the administration of the compound, the subject does not exhibit a severe toxicity following the administration of the T cell therapy. In some of any of the provided methods, the severe toxicity is severe cytokine release syndrome (CRS), optionally grade 3 or higher CRS, prolonged grade 3 or higher CRS, grade 4 CRS, or grade 5 CRS; and/or the severe toxicity is severe neurotoxicity, optionally grade 3 or higher neurotoxicity, prolonged grade 3 or higher neurotoxicity, grade 4 neurotoxicity, or grade 5 neurotoxicity. In some embodiments, the severe toxicity is severe CRS. In some embodiments, the severe toxicity is severe neurotoxicity.

In some of any of the provided methods, the administration of the compound is suspended and/or the dose of the compound is modified, optionally lowered, if the subject exhibits a toxicity following the administration of the compound, optionally a hematologic toxicity. In some of any of the provided methods, the administration of the compound is suspended if the subject exhibits a toxicity following the administration of the compound. In some of any of the provided methods, the dose of the compound is lowered if the subject exhibits a toxicity following the administration of the compound. In some of any of the provided methods, the toxicity is a hematological toxicity. In some of any of the provided methods, the toxicity is severe thrombocytopenia, optionally grade 4 thrombocytopenia or prolonged grade 4 thrombocytopenia. In some of any of the provided methods, the toxicity is severe neutropenia, optionally grade 4 neutropenia, prolonged grade 4 neutropenia, or febrile neutropenia, optionally grade 3 or higher febrile neutropenia or prolonged grade 3 or higher febrile neutropenia.

In some of any of the provided methods, the administration of the compound is restarted after the subject no longer exhibits the toxicity.

In some of any of the provided methods, the lymphoma expresses CD19.

In some of any of the provided methods, the lymphoma is a B-cell malignancy. In some of any of the provided methods, the lymphoma is a relapsed/refractory lymphoma. In some of any of the provided methods, the lymphoma is an aggressive lymphoma. In some of any of the provided methods, the lymphoma is a non-Hodgkin lymphoma (NHL), optionally wherein the NHL comprises aggressive NHL; diffuse large B cell lymphoma (DLBCL); DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally follicular lymphoma Grade 3B (FL3B); and/or high-grade B-cell lymphoma with MYC and BCL2 and/or BCL6 rearrangements with DLBCL histology (double/triple hit).

In some of any of the provided methods, the CD19 is a human CD19.

In some of any of the provided methods, the chimeric antigen receptor (CAR) comprises an extracellular antigen-recognition domain that specifically binds to the CD19 and an intracellular signaling domain comprising an ITAM. In some of any of the provided methods, the intracellular signaling domain comprises a signaling domain of a CD3-zeta (CD3ζ) chain, optionally a human CD3-zeta chain. In some of any of the provided methods, the intracellular signaling domain comprises a signaling domain of a human CD3-zeta (CD3ζ) chain. In some of any of the provided methods, the chimeric antigen receptor (CAR) further comprises a costimulatory signaling region. In some of any of the provided methods, the costimulatory signaling region comprises a signaling domain of CD28 or 4-1BB, optionally human CD28 or human 4-1BB. In some of any of the provided methods, the costimulatory signaling region comprises a signaling domain of human CD28. In some of any of the provided methods, the costimulatory signaling region comprises a signaling domain of human 4-1BB.

In some of any of the provided methods, the CAR comprises an scFv specific for the CD19; a transmembrane domain; a cytoplasmic signaling domain derived from a costimulatory molecule, which optionally is or comprises a 4-1BB, optionally human 4-1BB; and a cytoplasmic signaling domain derived from a primary signaling ITAM-containing molecule, which optionally is or comprises a CD3zeta signaling domain, optionally a human CD3zeta signaling domain; and optionally wherein the CAR further comprises a spacer between the transmembrane domain and the scFv. In some of any of the provided methods, the CAR comprises an scFv specific for the CD19; a transmembrane domain; a cytoplasmic signaling domain derived from human 4-1BB; a cytoplasmic signaling domain that is or comprises a human CD3zeta signaling domain; and a spacer between the transmembrane domain and the scFv.

In some of any of the provided methods, the CAR comprises, in order, an scFv specific for the CD19; a transmembrane domain; a cytoplasmic signaling domain derived from a costimulatory molecule, which optionally is or comprises a 4-1BB signaling domain, optionally a human 4-1BB signaling domain; and a cytoplasmic signaling domain derived from a primary signaling ITAM-containing molecule, which optionally is a CD3zeta signaling domain, optionally human CD3zeta signaling domain. In some of any of the provided methods, the CAR comprises, in order, an scFv specific for the CD19; a transmembrane domain; a cytoplasmic signaling domain that is or comprises a human 4-1BB signaling domain; and a cytoplasmic signaling domain that is a human CD3zeta signaling domain.

In some of any of the provided methods, the CAR comprises, in order, an scFv specific for the CD19; a spacer; a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain. In some of any of the provided methods, the CAR comprises, in order, an scFv specific for the CD19; a spacer; a transmembrane domain, a cytoplasmic signaling domain that is a 4-1BB signaling domain, and a cytoplasmic signaling domain that is or comprises a CD3zeta signaling domain.

In some of any of the provided methods, the spacer is a polypeptide spacer that comprises or consists of all or a portion of an immunoglobulin hinge or a modified version thereof or comprises about 15 amino acids or less. In some of any of the provided methods, the spacer comprises or consists of all or a portion of an immunoglobulin hinge, optionally an IgG4 hinge, or a modified version thereof and/or comprises about 15 amino acids or less. In some embodiments, the spacer comprises or consists of all or a portion of an immunoglobulin hinge, such as an IgG4, or a modified version thereof. In some of any of the provided methods, the spacer comprises or consists of all or a portion of an IgG4 hinge or a modified version thereof. In some of any of the provided methods, the spacer comprises about 15 amino acids or less. In some of any of the provided methods, the spacer is at or about 12 amino acids in length. In some of any of the provided methods, the spacer is at or about 12 amino acids in length. In some of any of the provided methods, the spacer has or consists of the sequence of SEQ ID NO: 1; a sequence encoded by SEQ ID NO: 2, SEQ ID NO: 30, SEQ ID NO: 31, SEQ ID NO: 32, SEQ ID NO: 33, SEQ ID NO: 34; or a variant of any of the foregoing having at least 85%, 86%, 87%, 88%, 89%, 90%, 91%, 92%, 93%, 94%, 95%, 96%, 97%, 98%, 99% or more sequence identity thereto. In some of any of the provided methods, the spacer has or consists of the sequence of SEQ ID NO: 1. In some embodiments, the spacer has a sequence encoded by SEQ ID NO: 2.

In some of any of the provided methods, the cytoplasmic signaling domain derived from a costimulatory molecule comprises the sequence set forth in SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto. In some of any of the provided methods, the cytoplasmic signaling domain derived from a primary signaling ITAM-containing molecule comprises the sequence of any of SEQ ID NO: 13-15 or a variant thereof having at least 85%, 86%, 87%, 88%, 89%, 90%, 91%, 92%, 93%, 94%, 95%, 96%, 97%, 98%, 99% or more sequence identity thereto.

In some of any of the provided methods, the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37); and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40). In some of any of the provided methods, the scFv comprises a CDRL1 sequence of RASQDISKYLN (SEQ ID NO: 35), a CDRL2 sequence of SRLHSGV (SEQ ID NO: 36), and a CDRL3 sequence of GNTLPYTFG (SEQ ID NO: 37); and/or a CDRH1 sequence of DYGVS (SEQ ID NO: 38), a CDRH2 sequence of VIWGSETTYYNSALKS (SEQ ID NO: 39), and a CDRH3 sequence of YAMDYWG (SEQ ID NO: 40). In some of any of the provided methods, the scFv comprises a CDRL1 sequence of RASQDISKYLN (SEQ ID NO: 35), a CDRL2 sequence of SRLHSGV (SEQ ID NO: 36), and a CDRL3 sequence of GNTLPYTFG (SEQ ID NO: 37); and a CDRH1 sequence of DYGVS (SEQ ID NO: 38), a CDRH2 sequence of VIWGSETTYYNSALKS (SEQ ID NO: 39), and a CDRH3 sequence of YAMDYWG (SEQ ID NO: 40). In some of any of the provided methods, the scFv comprises a variable light chain region comprising a CDRL1 sequence of FMC63, a CDRL2 sequence of FMC63, a CDRL3 sequence of FMC63, and a variable heavy chain region comprising a CDRH1 sequence of FMC63, a CDRH2 sequence of FMC63, and a CDRH3 sequence of FMC63. In some of any of the provided methods, the scFv comprises a variable heavy chain region of FMC63 and a variable light chain region of FMC63. In some embodiments, the scFv comprises a VH comprising the amino acid sequence set forth in SEQ ID NO: 41, and a VL comprising the amino acid sequence set forth in SEQ ID NO: 42. In some of any of the provided methods, the scFv comprises a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63. In some of any of the provided methods, the scFv comprises a variable heavy chain region of FMC63 and a variable light chain region of FMC63. In some of any of the provided methods, the scFv comprises a VH comprising SEQ ID NO: 41, and a VL comprising the amino acid sequence set forth as SEQ ID NO: 42. In some of any of the provided methods, the scFv has the sequence of amino acids set forth in SEQ ID NO: 43.

In some of any of the provided methods, the dose of engineered cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, or 5×10⁷ to 1×10⁸ total CAR-expressing T cells, each inclusive. In some of any of the provided methods, the dose of engineered cells comprises from or from about 1×10⁵ to 5×10⁸ total CAR-expressing T cells, inclusive. In some of any of the provided methods, the dose of engineered cells comprises from or from about 1×10⁶ to 2.5×10⁸ total CAR-expressing T cells, inclusive. In some of any of the provided methods, the dose of engineered cells comprises from or from about 5×10⁶ to 1×10⁸ total CAR-expressing T cells, inclusive. In some of any of the provided methods, the dose of engineered cells comprises from or from about 1×10⁷ to 2.5×10⁸ total CAR-expressing T cells, inclusive. In some of any of the provided methods, the dose of engineered cells comprises from or from about 5×10⁷ to 1×10⁸ total CAR-expressing T cells, inclusive.

In some of any of the provided methods, the dose of engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some of any of the provided methods, the dose of engineered cells comprises at least or at least about 1×10⁵ CAR-expressing cells. In some of any of the provided methods, the dose of engineered cells comprises at least or at least about 2.5×10⁵ CAR-expressing cells. In some of any of the provided methods, the dose of engineered cells comprises at least or at least about 5×10⁵ CAR-expressing cells. In some of any of the provided methods, the dose of engineered cells comprises at least or at least about 1×10⁶ CAR-expressing cells. In some of any of the provided methods, the dose of engineered cells comprises at least or at least about 2.5×10⁶ CAR-expressing cells. In some of any of the provided methods, the dose of engineered cells comprises at least or at least about 5×10⁶ CAR-expressing cells. In some of any of the provided methods, the dose of engineered cells comprises at least or at least about 1×10⁷ CAR-expressing cells. In some of any of the provided methods, the dose of engineered cells comprises at least or at least about 2.5×10⁷ CAR-expressing cells. In some of any of the provided methods, the dose of engineered cells comprises at least or at least about 5×10⁷ CAR-expressing cells. In some of any of the provided methods, the dose of engineered cells comprises at least or at least about 1×10⁸ CAR-expressing cells. In some of any of the provided methods, the dose of engineered cells comprises at least or at least about 2.5×10⁸ CAR-expressing cells. In some of any of the provided methods, the dose of engineered cells comprises at least or at least about 5×10⁸ CAR-expressing cells.

In some of any of the provided methods, the dose of engineered cells comprises at or about 1×10⁸ CAR-expressing T cells.

In some of any of the provided methods, the dose of engineered cells is administered parenterally, optionally intravenously. In some of any of the provided methods, the dose of engineered cells is administered intravenously.

In some of any of the provided methods, the T cells are primary T cells obtained from the subject. In some of any of the provided methods, the T cells are autologous to the subject. In some of any of the provided methods, the T cells are allogeneic to the subject.

In some of any of the provided methods, the dose of engineered cells comprises CD4+ T cells expressing the CAR and CD8+ T cells expressing the CAR and the administration of the dose comprises administering a plurality of separate compositions, said plurality of separate compositions comprising a first composition comprising one of the CD4+ T cells and the CD8+ T cells and a second composition comprising the other of the CD4+ T cells and the CD8+ T cells.

In some of any of the provided methods, the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or the initiation of administration of the first composition and the initiation of administration of the second composition are carried out between about 1 minute and about 1 hour apart or between about 5 minutes and about 30 minutes apart. In some of any of the provided methods, the first composition and second composition are administered no more than 2 hours, no more than 1 hour, no more than 30 minutes, no more than 15 minutes, no more than 10 minutes or no more than 5 minutes apart.

In some of any of the provided methods, the first composition comprises the CD4+ T cells. In some of any of the provided methods, the first composition comprises the CD8+ T cells.

In some of any of the provided methods, the first composition is administered prior to the second composition.

In some of any of the provided methods, prior to the administration of the T cell therapy, the subject has been preconditioned with a lymphodepleting therapy comprising the administration of fludarabine and/or cyclophosphamide. In some of any of the provided methods, the method further comprises, immediately prior to the administration of the T cell therapy, administering a lymphodepleting therapy to the subject comprising the administration of fludarabine and/or cyclophosphamide.

In some of any of the provided methods, the lymphodepleting therapy comprises administration of cyclophosphamide at about 200-400 mg/m², inclusive, optionally at or about 300 mg/m², and/or fludarabine at about 20-40 mg/m², optionally 30 mg/m², daily for 2-4 days, optionally for 3 days, or wherein the lymphodepleting therapy comprises administration of cyclophosphamide at about 500 mg/m². In some of any of the provided methods, the lymphodepleting therapy comprises administration of cyclophosphamide at or about 300 mg/m² and fludarabine at about 30 mg/m² daily for 3 days; and/or the lymphodepleting therapy comprises administration of cyclophosphamide at or about 500 mg/m² and fludarabine at about 30 mg/m² daily for 3 days.

In some of any of the provided methods, the subject is a human.

In some of any of the provided methods, at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or at least 60, 70, 80, 90, or 95% of subjects achieving a CR by six months remain in response, remain in CR, and/or survive or survive without progression, for at or greater than 3 months and/or at or greater than 6 months and/or at or greater than nine months; and/or at least 50%, at least 60% or at least 70% of the subjects treated according to the method achieve objective response (OR) optionally wherein the OR is durable, or is durable in at least 60, 70, 80, 90, or 95% of subjects achieving the OR, for at or greater than 6 months or at or greater than 9 months; and/or at least 60, 70, 80, 90, or 95% of subjects achieving an OR by six months remain in response or surviving for at or greater than 3 months and/or at or greater than 6 months.

In some of any of the provided methods, at or immediately prior to the time of the administration of the dose of engineered cells, the subject has relapsed following remission after treatment with, or become refractory to, one or more prior therapies for the NHL, optionally one, two, or three prior therapies other than another dose of engineered cells expressing the CAR. In some of any of the provided methods, at or immediately prior to the time of the administration of the dose of engineered cells, the subject has relapsed following remission after treatment with, or become refractory to, one prior therapy for the NHL. In some of any of the provided methods, at or immediately prior to the time of the administration of the dose of engineered cells, the subject has relapsed following remission after treatment with, or become refractory to, two prior therapies for the NHL. In some of any of the provided methods, at or immediately prior to the time of the administration of the dose of engineered cells, the subject has relapsed following remission after treatment with, or become refractory to, three prior therapies for the NHL. In some of any of the provided methods, the one or more prior therapies do not comprise another dose of engineered cells expressing the CAR.

In some of any of the provided methods, at or prior to the administration of the dose of engineered cells: the subject is or has been identified as having a double/triple hit lymphoma; the subject is or has been identified as having a chemorefractory lymphoma, optionally a chemorefractory DLBCL; and/or the subject has not achieved complete remission (CR) in response to a prior therapy. In some of any of the provided embodiments, the subject has not achieved complete remission (CR) in response to a prior therapy.

In some of any of the provided methods, the administration of the compound: reverses an exhaustion phenotype in CAR-expressing T cells in the subject; prevents, inhibits, or delays the onset of an exhaustion phenotype in CAR-expressing T cells in the subject; reduces the level or degree of an exhaustion phenotype in CAR-expressing T cells in the subject; or reduces the percentage or the total number of CAR-expressing T cells in the subject that have an exhaustion phenotype.

In some of any of the provided methods, the initiation of the administration of the compound is carried out subsequently to the administration of the T cell therapy and, following administration of the compound or initiation thereof, the subject exhibits a restoration or rescue of an antigen- or tumor-specific activity or function of the CAR-expressing T cells in said subject, optionally wherein said restoration, rescue, and/or initiation of administration of said compound, is at a point in time after CAR-expressing T cells in the subject or in the blood of the subject have exhibited an exhausted phenotype.

In some of any of the provided methods, the administration of the compound comprises administration at an amount, frequency and/or duration effective to: (a) effect an increase in antigen-specific or antigen receptor-driven activity of naïve or non-exhausted T cells in the subject, which optionally comprise T cells expressing said CAR, following exposure of the T cells to CD19 antigen or to an antigen receptor-specific agent as compared to the absence of said administration of said compound; or (b) prevent, inhibit or delay the onset of an exhaustion phenotype, in naïve or non-exhausted T cells in the subject, which optionally comprise T cells expressing said CAR, following exposure of the T cells to CD19 antigen or to an antigen receptor-specific agent, as compared to the absence of said administration of said compound; or (c) reverse an exhaustion phenotype in exhausted T cells, optionally comprising T cells expressing said CAR, in the subject, as compared to the absence of said administration of said compound.

In some of any of the provided methods, the administration of the compound comprises administration at an amount, frequency and/or duration effective (i) to effect said increase in activity and (ii) to prevent, inhibit or delay said onset of said exhaustion phenotype and/or reverse said exhaustion phenotype.

In some of any of the provided methods, the T cells in the subject comprise T cells expressing said CAR and/or said antigen is CD19, e.g., the exposure is to CD19 antigen.

In some of any of the provided methods, the exhaustion phenotype, with reference to a T cell or population of T cells, comprises: an increase in the level or degree of surface expression on the T cell or T cells, or in the percentage of said population of T cells exhibiting surface expression, of one or more exhaustion markers, optionally 2, 3, 4, 5 or 6 exhaustion markers, compared to a reference T cell population under the same conditions; or a decrease in the level or degree of an activity exhibited by said T cells or population of T cells upon exposure to a CD19 antigen or antigen receptor-specific agent, compared to a reference T cell population, under the same conditions.

In some of any of the provided methods, the increase in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more. In some of any of the provided methods, the decrease in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

In some of any of the provided methods, the reference T cell population is a population of T cells known to have a non-exhausted phenotype, is a population of naïve T cells, is a population of central memory T cells, or is a population of stem central memory T cells, optionally from the same subject, or of the same species as the subject, from which the T cell or T cells having the exhaustion phenotype are derived. In some of any of the provided methods, the reference T cell population (a) is a subject-matched population comprising bulk T cells isolated from the blood of the subject from which the T cell or T cells having the exhaustion phenotype is derived, optionally wherein the bulk T cells do not express the CAR and/or (b) is obtained from the subject from which the T cell or T cells having the exhaustion phenotype is derived, prior to receiving administration of a dose of T cells expressing the CAR. In some of any of the provided methods, the reference T cell population is a composition comprising a sample of the T cell therapy, e.g., a sample of T cells expressing the CAR, or pharmaceutical composition comprising T cells expressing the CAR, prior to its administration to the subject, optionally wherein the composition is a cryopreserved sample.

In some of any of the provided methods, the one or more exhaustion markers is an inhibitory receptor. In some of any of the provided methods, the one or more exhaustion markers is selected from among PD-1, CTLA-4, TIM-3, LAG-3, BTLA, 2B4, CD160, CD39, VISTA, and TIGIT.

In some of any of the provided methods, the activity is one or more of proliferation, cytotoxicity or production of one or a combination of inflammatory cytokines, optionally wherein the one or a combination of cytokines is selected from the group consisting of IL-2, IFN-gamma and TNF-alpha.

In some of any of the provided methods, the exposure to said CD19 antigen or antigen receptor-specific agent comprises incubation with the CD19 antigen or antigen receptor-specific agent, optionally an agent that binds the antigen-binding domain of the CAR. In some of any of the provided methods, the exposure to the CD19 antigen or antigen receptor-specific agent comprises exposing the T cells to CD19 antigen-expressing target cells, optionally cells of the B cell malignancy.

Also provided are combination therapies comprising a T cell therapy comprising a dose of engineered cells comprising T cells expressing a chimeric antigen receptor (CAR) that targets a cancer expressing CD19 and a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof for use in a method of treating the cancer in accord with any of the provided methods.

Also provided is a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof for use in a method of treating a cancer expressing CD19 in accord with any of the provided methods.

Also provided are T cell therapies comprising a dose of engineered cells comprising T cells expressing a chimeric antigen receptor (CAR) that targets a cancer expressing CD19 for use in a method of treating the cancer in accord with any of the provided methods.

Also provided are uses of a combination therapy comprising a T cell therapy comprising a dose of engineered cells comprising T cells expressing a chimeric antigen receptor (CAR) that targets a cancer expressing CD19 and a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof for treating the cancer in accord with any of the provided methods.

Also provided are uses of a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof for treating a cancer expressing CD19 in accord with any of the provided methods.

Also provided are uses of a T cell therapy comprising a dose of engineered cells comprising T cells expressing a chimeric antigen receptor (CAR) that targets a cancer expressing CD19 for treating the cancer in accord with any of the provided methods.

Also provided are uses of a combination therapy comprising a T cell therapy comprising a dose of engineered cells comprising T cells expressing a chimeric antigen receptor (CAR) that targets a cancer expressing CD19 and a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof for the manufacture of a medicament for treating the cancer in accord with any of the provided methods.

Also provided are uses of a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt, solvate, hydrate, co-crystal, clathrate, or polymorph thereof for the manufacture of a medicament for treating a cancer expressing CD19 in accord with any of the provided methods.

Also provided are uses of a T cell therapy comprising a dose of engineered cells comprising T cells expressing a chimeric antigen receptor (CAR) that targets a cancer expressing CD19 for the manufacture of a medicament for treating the cancer in accord with any of the provided methods.

In some of any of the provided embodiments, the CAR binds cluster of differentiation 19 (CD19). In some embodiments, the cancer expressing CD19 is a lymphoma.

In some of any of the provided embodiments, the compound is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof.

### BRIEF DESCRIPTION OF THE DRAWINGS

FIG. 1 shows expression levels of Aiolos and Ikaros transcription factors in anti-CD19 CAR T cells following stimulation with a CAR-specific anti-idiotypic antibody in the presence of various immunomodulatory compounds.

FIG. 2A shows the percentage of live anti-CD19 CAR T cells following stimulation with a CAR-specific anti-idiotypic antibody in the presence of various immunomodulatory compounds.

FIGS. 2B and 2C show the number of cell doublings of anti-CD19 CAR T cells following stimulation with a CAR-specific anti-idiotypic antibody in the presence of various immunomodulatory compounds.

FIGS. 2D and 2E show the percentage of anti-CD19 CAR T cells in G1 phase of the cell cycle following stimulation with a CAR-specific anti-idiotypic antibody in the presence of various immunomodulatory compounds.

FIG. 2F shows cytokine levels of anti-CD19 CAR T cells following stimulation with a CAR-specific anti-idiotypic antibody in the presence of various immunomodulatory compounds. Log₂ fold-change values greater than zero are indicated with a “+”.

FIG. 2G shows cell number of RL tumor cells treated with anti-CD19 CAR T cells and Compound C.

FIG. 2H shows cell number of RL tumor cells treated with anti-CD19 CAR T cells and Compound 2.

FIG. 3A shows cytokine production of anti-CD19 CAR T cells following chronic stimulation.

FIG. 3B shows cytolytic function of anti-CD19 CAR T cells following chronic stimulation.

FIG. 3C shows representative images of CD19-expressing tumor spheroids at day 9 of co-culture with anti-CD19 CAR T cells that were stimulated in the presence of Compound C (concurrent treatment).

FIGS. 3D and 3E show the volume of CD19-expressing tumor spheroids co-cultured with anti-CD19 CAR T cells that were stimulated in the presence of Compound C after chronic stimulation (concurrent treatment).

FIG. 3F shows IFNγ concentration in the supernatant of the co-cultured cells.

FIG. 3G shows Ikaros expression in anti-CD19 CAR T cells after six days of chronic stimulation in the presence of Compound C or Compound 2.

FIG. 3H depicts volcano plots showing differentially expressed genes in chronically stimulated anti-CD19 CAR T cells concurrently treated with Compound C.

FIG. 3I shows a comparison of effects on gene expression profile (log 2-fold changes) induced by Compound C concurrent treatment on anti-CD19 CAR T cells that had been chronically stimulated.

FIG. 3J shows KEGG pathway enrichment analysis of differentially expressed genes in chronically stimulated anti-CD19 CAR T cells after concurrent treatment with Compound C.

FIG. 4A shows representative images of CD19-expressing tumor spheroids at day 9 of co-culture with anti-CD19 CAR T cells in the presence of Compound C (rescue treatment).

FIGS. 4B and 4C show the volume of CD19-expressing tumor spheroids co-cultured with anti-CD19 CAR T cells in the presence of Compound C (rescue treatment).

FIG. 4D shows IFNγ concentration in the supernatant of the co-cultured cells.

FIG. 4E depicts volcano plots showing differentially expressed genes in chronically stimulated anti-CD19 CAR T cells following rescue treatment with Compound C.

FIG. 4F shows a comparison of effects on gene expression profile (log 2-fold changes) induced by Compound C rescue treatment on anti-CD19 CAR T cells that had been chronically stimulated.

FIG. 4G shows KEGG pathway enrichment analysis of differentially expressed genes in chronically stimulated anti-CD19 CAR T cells after rescue treatment with Compound C.

FIG. 5A shows tumor cell number during re-challenge of CAR T cells chronically stimulated in the presence of Compound C (concurrent treatment).

FIG. 5B shows tumor cell number during re-challenge of CAR T cells in the presence of Compound C (rescue treatment).

FIG. 6A shows expected plasma concentration levels of Compound C based on a once every seven days (Q7D) dosing regimen.

FIGS. 6B and 6C show Ikaros levels in anti-CD19 CAR T cells following 24-hour (FIG. 6B) or six-day exposure (FIG. 6C) to Compound C.

FIG. 6D shows predicted absolute neutrophil count (ANC) profiles based on a Q7D dosing regimen of Compound C, as generated using four different pharmacodynamic models.

FIG. 7A shows tumor cell number during re-challenge of CAR T cells chronically stimulated in the presence of Compound C, including for CAR T cells that were chronically stimulated in the presence of Compound C for one day at a high concentration of Compound C, then for five days at a low concentration of Compound C.

FIGS. 7B and 7C show cytolytic activity and CD27/CCR7 expression, respectively, of CAR T cells chronically stimulated in the presence of Compound C, including for CAR T cells that were chronically stimulated in the presence of Compound C for one day at a high concentration of Compound C, then for six days at a low concentration of Compound C.

### DETAILED DESCRIPTION

Provided herein are methods and uses of a cell therapy, such as a cell therapy containing engineered T cells (e.g., CAR-T cells) and a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

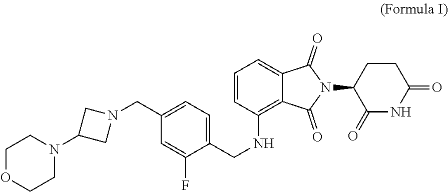 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, stereoisomer, tautomer, or racemic mixtures thereof (Compound C), and compositions thereof, for the treatment of subjects with a cancer or proliferative disease. In some aspects, the cell therapy is a T cell therapy. In some aspects, the cell therapy is an adoptive T cell therapy comprising T cells that specifically recognize and/or target an antigen associated with the cancer or proliferative disease, such as an antigen associated with a B cell malignancy, e.g., a lymphoma such as Non Hodgkin Lymphoma (NHL) or a subtype thereof. In some aspects, the cell therapy includes T cells engineered with a chimeric antigen receptor (CAR) comprising an antigen binding domain that binds, such as specifically binds, to the antigen. In some cases, the antigen targeted by the cell therapy is CD19. In some aspects, the cancer, e.g., the lymphoma, expresses CD19. Also provided herein are combinations and articles of manufacture, such as kits, that contain a composition comprising the cell therapy and/or a composition comprising Compound C, and uses of such compositions and combinations to treat or prevent diseases, conditions, and disorders, including cancers, such as a lymphoma.

(S)-2-(2,6-dioxopiperidin-3-yl)-4-((2-fluoro-4-((3-morpholinoazetidin-1-yl)methyl)benzyl)amino)isoindoline-1,3-dione (Compound C) is a an oral cereblon-modulating agent (CELMOD). Cereblon functions as a substrate receptor for a CRL4 ubiquitin E3 ligase, and the binding of cereblon-modulating compounds induces the recruitment, ubiquitination, and destruction of key target substrates, such as Ikaros family zinc finger proteins 1 and 3 (IKZF1 and IKZF3, also known as Ikaros and Aiolos, respectively), to mediate cellular effects. As shown herein, the administration of Compound C leads to profound degradation of Ikaros and Aiolos, more so than with other CELMoDs in its class (e.g., lenalidomide, avadomide, and iberdomide). However, a potential side effect of Ikaros and Aiolos degradation is the development of neutropenia secondary to neutrophil maturation arrest. Neutropenia can develop as a consequence of the depletion of Ikaros, a critical transcriptional factor controlling hematopoiesis, and is due to late-stage maturation arrest of neutrophil progenitors

Cell therapies, such as T cell-based therapies, for example adoptive T cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of diseases and disorders such as B cell malignancy. The engineered expression of recombinant receptors, such as chimeric antigen receptors (CARs), on the surface of T cells enables the redirection of T-cell specificity. In clinical studies, CAR-T cells, for example anti-CD19 CAR-T cells, have produced durable, complete responses in both leukemia and lymphoma patients (Porter et al. (2015) Sci Transl Med., 7:303ra139; Kochenderfer (2015) J. Clin. Oncol., 33: 540-9; Lee et al. (2015) Lancet, 385:517-28; Maude et al. (2014) N Engl J Med, 371:1507-17).

In certain contexts, available approaches for adoptive cell therapy may not always be entirely satisfactory. For example, although CAR T cell persistence can be detected in many subjects with lymphoma, fewer complete responses (CRs) have been observed in subjects with NHL compared to subjects with ALL. More specifically, while higher overall response rates of up to 80% (CR rate 47% to 60%) have been reported after CAR T cell infusion, responses in some are transient, and subjects have been shown to relapse in the presence of persistent CAR T cells (Neelapu, 58th Annual Meeting of the American Society of Hematology (ASH): 2016; San Diego, CA, USA. Abstract No. LBA-6.2016; Abramson, Blood. 2016 Dec. 1; 128(22):4192). Another study reported a long-term CR rate of 40% (Schuster, Ann Hematol. 2016 October; 95(11):1805-10).

In some aspects, an explanation for this is the immunological exhaustion of circulating CAR-expressing T cells and/or changes in T lymphocyte populations. This is because, in some contexts, optimal efficacy can depend on the ability of the administered cells to have the capability to become activated, to expand, to exert various effector functions, including cytotoxic killing and secretion of various factors such as cytokines, to persist, including long-term, to differentiate, transition, or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and to avoid or reduce exhaustion, anergy, peripheral tolerance, terminal differentiation, and/or differentiation into a suppressive state.

In some aspects, the exposure, persistence, and functions of engineered cells is reduced or declines after administration to the subject. In some aspects, following long-term stimulation or exposure to antigen and/or exposure under conditions in the tumor microenvironment, T cells can over time become hypofunctional and/or exhibit features associated with exhausted state. In some aspects, this reduces the persistence and efficacy of the T cells against antigen and limits their ability to be effective. Yet, observations indicate that, in some cases, the administered cells expressing the recombinant receptors can re-expand and/or be re-activated in vivo (e.g., show increased number of cells or duration over time) to improve efficacy and therapeutic outcomes in adoptive cell therapy. There is a need for methods to improve the efficacy and function of CAR T cells, particularly to minimize, reduce, prevent, or reverse hypofunctional or exhausted states.

The provided methods are based on observations that treatment with Compound C can improve T cell function, including functions related to the ability to produce one or more cytokines, cytotoxicity, expansion, proliferation, and persistence of T cells. In some aspects, the provided methods enhance or modulate proliferation and/or activity of T cell activity associated with administration of a cell therapy (e.g. CAR-expressing T cells). It is found that such methods and uses provide for or achieve improved or greater T cell functionality, and thereby improved anti-tumor efficacy.

It also is found herein that, in addition to potentiating T cell function, treatment with Compound C can reverse, delay, or prevent T cell exhaustion, including by increasing T cell signaling and/or altering one or more genes that are differentially regulated following chronic stimulation. While, in some cases, agents that increase or potentiate T cell activity may drive the cells to an exhausted state, it is found herein that activity of Compound C to exert a potentiating effect on T cell activity is decoupled from T cell exhaustion. Moreover, observations herein show that Compound C exhibits activity to rescue T cells from T cell exhaustion, such as by restoring or partially restoring one or more T cell activities after a cell has shown features of exhaustion. Remarkably, results herein show that T cells that have been chronically stimulated and exhibit features of exhausted T cells are able to recover activity or have their activity restored or partially restored following exposure to Compound C. The observations herein support that the provided methods may also achieve improved or more durable responses as compared to certain alternative methods, such as in particular groups of subjects treated.

It also is shown herein that low-dose Compound C treatment (e.g., 1 nM) is able to profoundly degrade both Ikaros and Aiolos expression in chronically stimulated CAR T cells. However, it is also shown herein that higher doses of Compound C (e.g., 10 and 100 nM) can lead to sustained target degradation of Ikaros and Aiolos and result in reduced cytolytic function. Such results suggest that excessive, sustained target degradation of Ikaros and Aiolos may have a counter-productive effect and result in CAR T cell dysfunction. Based on these results, and as Compound C can more profoundly degrade Ikaros and Aiolos than can other CELMOD compounds that target Ikaros and Aiolos, it was hypothesized herein that a single weekly dose would be sufficient to effect deep, but transient degradation of Ikaros and Aiolos, thus potentially allowing for recovery of Ikaros and/or Aiolos expression between doses. As shown herein, pharmacokinetic modeling of Compound C at a weekly dose (Q7D) of 0.3 mg demonstrated rapid Cₘₐₓ (approximately 20 nM) and biphasic elimination of Compound C, with a Cₘᵢₙ on Day 7 of approximately 1 nM. Modeling also showed that a weekly dose of 0.3 mg would not substantially affect neutrophil count. Also as shown herein in pre-clinical models, anti-CD19 CAR T cytolytic activity against CD19+ target cells was most pronounced following exposure to transient (one-day) high concentrations of Compound C (20-100 nM) followed by wash-out and chronic low concentrations of Compound C (five days at 1 nM), this high-low concentration exposure being selected to model the pharmacokinetic profile of Compound C at 0.3 mg once every seven days (Q7D). Conversely, higher chronic concentrations of Compound C had detrimental effects on cytolytic function.

Thus, based on the results shown herein, cell therapies, e.g., CAR T cell therapies, in combination with intermittent (e.g., once every seven days) dosing of Compound C may provide a useful therapeutic approach for preventing premature exhaustion of CAR T cells, enhancing and prolonging T cell functionality, and leading to a more profound and durable response in cancers like lymphoma, while also reducing risk of patient development of neutropenia secondary to neutrophil maturation arrest.

In some embodiments, T cell function (including functions related to the expansion, proliferation, and persistence of T cells) of an engineered T cell therapy administered in accordance with the provided methods are improved by Compound C. In some embodiments, the methods are advantageous by virtue of administering T cell therapy, such as a composition including cells for adoptive cell therapy, e.g., such as a T cell therapy (e.g. CAR-expressing T cells) in combination with Compound C. In some aspects, the provided methods and uses provide for or achieve improved or more durable responses or efficacy as compared to certain alternative methods. In some aspects, the provided methods enhance or modulate proliferation and/or activity of T cell activity associated with administration of the T cell therapy (e.g. CAR-expressing T cells). In particular embodiments, combination therapy with Compound C may provide a useful therapeutic approach for enhancing and prolonging the activity of CAR T cells across B cell malignancies by modulating the tumor microenvironment, by improving persistent anti-tumor function of CAR T cells.

In some embodiments, Compound C is administered to the subject a sufficient time after receiving a lymphodepleting therapy, such that myelosuppressive effects of Compound C and the lymphodepleting therapy are minimized.

In some embodiments, the provided methods are used at a time at which a T cell therapy (e.g. CAR T cells) may exhibit or are likely to exhibit features of exhaustion. In some embodiments, an exhaustion phenotype is evident after T cells, having reached peak expansion, begin to decline in number in the blood of the subject. In some embodiments, the methods of exposing or contacting T cells of a T cell therapy (CAR T cells) with Compound C are carried out at a time at which the T cells exhibit an increase in a hypofunctional or exhausted state compared to at the time just prior to exposure of the T cells to an antigen (baseline) or to a time point at which the cells have been exposed to the antigen but are continuing to proliferate and have not yet reached peak expansion. In some embodiments, an increase in hypofunctional or exhausted state can be determined by increased expression of an exhaustion marker compared to the previous earlier timepoint. In some embodiments, the increase in the hypofunctional or exhausted state, such as increase in expression of an exhaustion marker, is at a time following administration of the T cell therapy (e.g. CAR T cells) to a subject having a disease or condition associated with the antigen targeted by the T cell therapy. The T cells, such as T cells in peripheral blood after administration to a subject, can be monitored for markers of T cell activation or exhaustion such as PD-1, TIM-3, and LAG-3.

In some embodiments, the provided methods call for the administration of a T cell therapy, e.g. CAR T cells, and the initiation of administration of Compound C at a time prior to the CAR T cells exhibiting or being likely to exhibit an exhaustion phenotype. In some embodiments, the administration of Compound C is initiated at a timepoint at which CAR T cells are still expanding or are still capable of expanding. In some embodiments, the administration of Compound C is initiated at a timepoint prior to or suspected to be prior to the presence of peak CAR T cell numbers in the blood of the subject. In some aspects, the initiation of Compound C administration at this timepoint potentiates CAR T cell function. In some aspects, the initiation of Compound C administration at this timepoint also delays or prevents CAR T cell exhaustion.

In some embodiments, the administration of Compound C is initiated at a time that is or that is suspected or likely to be before or about at a time peak CAR-T cells are present in the blood of the subject, e.g. within 21 days after initiation of administration of the T cell. In some cases, peak CAR-T cells present within 11-15 days following administration of CAR T cells. In some embodiments, the administration of Compound C is initiated at a time that is 1 to 15 days, e.g. at or about 1 day or 8 days or 15 days after initiation of administration of the cell therapy. In some embodiments, Compound C is administered at a time when the subject does not exhibit a severe toxicity following the administration of the cell therapy.

In some embodiments, the provided methods do not result in a high rate or likelihood of toxicity or toxic outcomes, or reduces the rate or likelihood of toxicity or toxic outcomes, such as neurotoxicity (NT), cytokine release syndrome (CRS), or hematological toxicities, such as neutropenia, such as compared to certain other cell therapies or immunomodulatory drug regimens.

In some embodiments, the methods do not result in, or do not increase the risk of, certain hematological toxicities, such as neutropenia or thrombocytopenia. In some embodiments, no more than 50% of subjects exhibit a neutropenia higher than grade 3, such as a prolonged grade 3 neutropenia or a grade 4 neutropenia, and/or a thrombocytopenia higher than grade 3, such as a grade 3 or grade 4 thrombocytopenia. In some embodiments, at least 50% of subjects treated according to the method (e.g. at least 60%, at least 70%, at least 80%, at least 90% or more of the subjects treated) do not exhibit a severe neutropenia or a severe thrombocytopenia of grade 3 or higher than grade 3.

In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxicity. In some embodiments, no more than 50% of subjects treated (e.g. at least 60%, at least 70%, at least 80%, at least 90% or more of the subjects treated) exhibit a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells.

In some aspects, the dose, timing of doses, or number of doses is not expected to cause severe toxicity, such as Grade 4 neutropenia or Grade 4 thrombocytopenia. In some aspects, the provided methods minimize or avoid toxicity following administration of the T cell therapy and/or Compound C to a subject, for instance throughout intermittent dosing of Compound C. In some aspects, the methods provided herein involve administering doses that are substantially lower, and given at a reduced frequency, than doses that may be used for Compound C in monotherapy approaches. In some aspects, the methods provided herein involve administering fewer doses than may be used for Compound C in monotherapy approaches. In some aspects, the methods provided herein involve administering intermittent doses of Compound C, with longer time periods between doses of Compound C than may be used for Compound C in monotherapy approaches.

In some cases, Compound C is administered at a time that it can efficiently/effectively boost or prime the cells. In some embodiments, the administration of Compound C is initiated at or before peak or maximum level of the cells of the cell therapy is detectable in the blood of the subject. In some embodiments, the provided methods can potentiate T cell therapy, e.g. CAR-T cell therapy, which, in some aspects, can improve outcomes for treatment. In some embodiments, the methods are particularly advantageous in subjects in which the cells of the T cell therapy exhibit weak expansion, have become exhausted, exhibit a reduced or decreased persistence in the subject and/or in subjects that have a cancer that is resistant or refractory to other therapies, and/or is an aggressive or high-risk cancer.

In some embodiments, a subject having received administration of a T cell therapy, e.g. CAR-T cell, is monitored for the presence, absence or level of T cells of the therapy in the subject, such as in a biological sample of the subject, e.g. in the blood of the subject. In some embodiments, the provided methods result in genetically engineered cell with increased persistence and/or better potency in a subject to which it is administered. In some embodiments, the persistence of genetically engineered cells, such as CAR-expressing T cells, in the subject is greater as compared to that which would be achieved by alternative methods, such as those involving administration of a T cell therapy but in the absence of administration of Compound C. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the recombinant receptor (e.g., CAR-expressing cells) in the blood or serum or organ or tissue (e.g., disease site) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the recombinant receptor (e.g. CAR-expressing cells) can be used to distinguish the administered cells from endogenous cells in a subject.

In some embodiments, Compound C is administered for a period of time to enhance, increase, or optimize durability of response. In some aspects, the provided methods are based on observations that subjects who achieve or are in complete remission (CR) at 3 months are more likely to sustain the response longer term, such as survive or survive without progression for greater than or greater than about three months, four months, five months, six months, seven months, eight months, nine months, ten months, eleven months or twelve months after ending the treatment or after first achieving a complete response (CR) following administration of the combination therapy. In some aspects, the methods are carried out to administer Compound C, such as in a particular intermittent dosing regimen as described, for a period of time that is at least 3 months or about three months after initiation of administration of the T cell therapy.

In some aspects, the provided methods and uses provide for or achieve improved or more durable responses or efficacy as compared to certain alternative methods, e.g. methods that include administration of the T cell therapy or Compound C as a monotherapy or without administration as a combination therapy together as described herein, such as in particular groups of subjects treated. In some embodiments, the methods are advantageous by virtue of administering T cell therapy, such as a composition including cells for adoptive cell therapy, e.g., such as a T cell therapy (e.g. CAR-expressing T cells), and Compound C. In some embodiments, such responses are observed in high risk patients with poor prognosis, such as those having high-risk disease, e.g., high-risk NHL. In some aspects, the methods treat subjects having a form of aggressive and/or poor prognosis B-cell non-Hodgkin lymphoma (NHL), such as NHL that has relapsed or is refractory (R/R) to standard therapy or has a poor prognosis. In some embodiments, subjects treated according to the provided methods have diffuse large B-cell lymphoma (DLBCL) or follicular lymphoma.

In some embodiments, at least 35%, at least 40%, at least 50%, at least 55%, at least 60%, at least 65%, at least 70%, or at least 75% or more of the subjects treated according to the provided methods, and/or with the provided articles of manufacture, kits or compositions, achieve a complete response (CR). In some embodiments, the subject is in CR and exhibits minimum residual disease (MRD). In some embodiments, the subject is in CR and is MRD−. In some embodiments, at least 50%, at least 60%, at least 70%, at least 80%, or at least 90% of the subjects treated according to the provided methods, and/or with the provided articles of manufacture, kits or compositions, achieve an objective response of a partial response (PR). In some embodiments, at least 60%, at least 70%, at least 80%, at least 90%, at least 95% or more of the subjects treated according to the provided methods, and/or with the provided articles of manufacture, kits or compositions, achieve a CR or PR at six months, at seven months, at eight months, at nine months, at ten months, at eleven months or a year after initiation of administration of the cell therapy.

In some embodiments, by three months, four months, five months, six months, seven months, eight months, nine months, ten months, eleven months or twelve months or more after initiation of administration of the cell therapy, at least 60%, at least 70%, at least 80%, at least 90%, at least 95% or more of the subjects treated according to the provided methods, and/or with the provided articles of manufacture, kits or compositions, remain in response, such as remain in CR or an objective response (OR). In some embodiments, such response, such as CR or OR, is durable for at least three months, four months, five months, six months, seven months, eight months, nine months, ten months, eleven months, twelve months or more such as in at least or about at least 60%, at least 70%, at least 80%, at least 90%, at least 95% or more of the subjects treated according to the provided methods or in such subjects who achieve a CR by three months, four months, five months or six months. In some embodiments, at least 60%, at least 70%, at least 80%, at least 90%, at least 95% or more of the subjects treated according to the provided methods, and/or with the provided articles of manufacture, kits or compositions, or such subjects who achieve a CR by three months, four months, five months or six months survive or survive without progression for greater than or greater than about six months, seven months, eight months, nine months, ten months, eleven months, twelve months or longer.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. COMBINATION THERAPY

Provided herein are methods and uses of engineered cells, such as T cells (e.g., CAR-T cells) and (S)-2-(2,6-dioxopiperidin-3-yl)-4-((2-fluoro-4-((3-morpholinoazetidin-1-yl)methyl)benzyl)amino)isoindoline-1,3-dione or a compound of Formula I

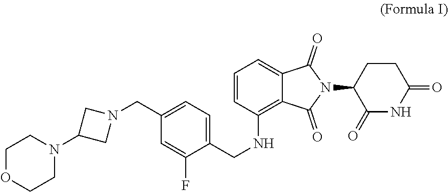 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, stereoisomer, tautomer or racemic mixtures thereof (Compound C), including compositions thereof, for the treatment of subjects with cancer. In some embodiments, the methods are for treating a subject with a lymphoma. In some embodiments, the lymphoma is a B cell malignancy. In some aspects, the lymphoma is a non-Hodgkin lymphoma (NHL). In some embodiments, the cancer, e.g., the lymphoma, expresses CD19. In some aspects, the methods and uses provide for or achieve improved response and/or more durable responses or efficacy, e.g., in particular groups of subjects treated, as compared to certain alternative methods.

Methods of preparing Compound C are described in US 2019/0322647, which is incorporated herein by reference in its entirety.

In some embodiments, the methods and uses include 1) administering to the subject a cell therapy that includes T cells expressing genetically engineered cell surface receptors (e.g., recombinant antigen receptors), for instance chimeric receptors such as chimeric antigen receptors (CARs), that recognize an antigen expressed by, associated with, and/or specific to the lymphoma (e.g., B cell malignancy, such as NHL) and/or cell type from which the lymphoma is derived, and 2) administering to the subject Compound C. In some embodiments, administration of Compound C is initiated after (subsequently to) administering the cell therapy or after (subsequently) to initiating administration of the cell therapy. In some cases, Compound C is administered to a subject that has received administration of a cell therapy. The methods can involve administering one or more doses of engineered cells and more than one dose of Compound C to the subject.

The combination therapy, e.g., including engineered cells expressing a recombinant receptor, such as a chimeric antigen receptor (CAR) and Compound C or compositions comprising the engineered cells and/or Compound C described herein are useful in a variety of therapeutic, diagnostic and prophylactic indications. For example, the combinations are useful in treating a variety of diseases and disorders in a subject. Such methods and uses include therapeutic methods and uses, for example, involving administration of the engineered cells, Compound C and/or compositions containing one or both, to a subject having a disease, condition, or disorder, such as a tumor or cancer. In some embodiments, the engineered cells, Compound C and/or compositions containing one or both are administered in an effective amount to effect treatment of the disease or disorder. Uses include uses of the engineered cells, Compound C and/or compositions containing one or both in such methods and treatments, and in the preparation of a medicament in order to carry out such therapeutic methods. In some embodiments, the methods are carried out by administering the engineered cells, Compound C, and/or compositions containing one or both, to the subject having or suspected of having the disease or condition. In some embodiments, the methods thereby treat the disease or condition or disorder in the subject. In some embodiments, the engineered cells are any as described in Section II.

In some embodiments, the combination therapy is administered to a subject having a lymphoma. In some embodiments, the combination therapy is administered to a subject having a particular B cell malignancy. The B cell malignancy that is treated can be any in which expression of an antigen is associated with and/or involved in the etiology of the B cell malignancy, e.g. causes, exacerbates or otherwise is involved in the B cell malignancy. Exemplary B cell malignancies can include diseases or conditions associated with malignancy or transformation of cells (e.g. a cancer). Exemplary antigens, which include antigens associated with various B cell malignancies that can be treated, are described herein. In particular embodiments, the chimeric antigen receptor specifically binds to an antigen associated with the disease or condition. In some embodiments, antigens targeted by the receptors include antigens associated with a B cell malignancy, such as any of a number of known B cell marker. In some embodiments, the antigen is expressed by or on B cells, including human B cells. In some embodiments, the antigen targeted by the receptor is CD20, CD19, CD22, ROR1, CD45, CD21, CD5, CD33, Igkappa, Iglambda, CD79a, CD79b or CD30. In some embodiments, the antigen is CD19 and the chimeric antigen receptor specifically binds CD19. In some embodiments, the CD19 antigen is a human CD19. In some embodiments, the lymphoma includes B cells expressing CD19. It is understood that description of any of the methods provided herein in which the CAR-expressing T cells are specific to CD19 also can be carried out by targeting of another B cell antigen or an antigen associated with or expressed on cells of a T cell malignancy, such as any described above.

In some embodiments, the B cell malignancy to be treated include leukemia and lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, and diffuse large B-cell lymphoma (DLBCL). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histiocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the methods involve treating a subject having a lymphoma, such as a non-Hodgkin lymphoma (NHL) by administering antigen receptor-expressing cells (e.g. CAR-expressing cells) and Compound C. In some embodiments, Compound C is administered after or subsequent to administering the recombinant receptor-expressing cells (e.g. CAR-expressing cells), such as after or subsequent to initiating administration of the recombinant receptor-expressing cells (e.g. CAR-expressing cells).

In some embodiments, NHL can be staged based on the Lugano classification (see, e.g., Cheson et al., (2014) JCO 32(27):3059-3067; Cheson, B. D. (2015) Chin Clin Oncol 4(1):5). In some cases, the stages are described by Roman numerals I through IV (1-4), and limited stage (I or II) lymphomas that affect an organ outside the lymph system (an extranodal organ) are indicated by an E. Stage I represents involvement in one node or a group of adjacent nodes, or a single extranodal lesions without nodal involvement (IE). Stage 2 represents involvement in two or more nodal groups on the same side of the diaphragm or stage I or II by nodal extent with limited contiguous extranodal involvement (IIE). Stage III represents involvement in nodes on both sides of the diaphragm or nodes above the diaphragm with spleen involvement. Stage IV represents involvement in additional non-contiguous extralymphatic involvement. In addition, “bulky disease” can be used to describe large tumors in the chest, in particular for stage II. The extent of disease is determined by positron emission tomography (PET)-computed tomography (CT) for avid lymphomas, and CT for non-avid histologies.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). In some embodiments, the subject has an ECOG status of less than or equal to 1. The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of selfcare; see e.g., Sørensen et al., (1993) Br J Cancer 67(4) 773-775. The criteria reflective of the ECOG performance status are described in Table 1 below:

TABLE 1
ECOG Performance Status Criteria

| Grade | ECOG performance status |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | e.g., light house work, office work |
| 2 | Ambulatory and capable of all selfcare but unable to carry |
| | out any work activities; up and about more than 50% of |
| | waking hours |
| 3 | Capable of only limited selfcare; confined to bed or chair |
| | more than 50% of waking hours |
| 4 | Completely disabled; cannot carry on any selfcare; totally |
| | confined to bed or chair |
| 5 | Dead |

In some embodiments, the subject has or has been identified as having a double/triple hit lymphoma or a lymphoma of the double/triple hit molecular subtypes. In some embodiments, the lymphoma is a double hit lymphoma characterized by the presence of MYC (myelocytomatosis oncogene), BCL2 (B-cell lymphoma 2), and/or BCL6 (B-cell lymphoma 6) gene rearrangements (e.g., translocations). In some embodiments, the lymphoma is a triple hit lymphoma characterized by the presence of MYC, BCL2, and BCL6 gene rearrangements; see, e.g., Aukema et al., (2011) Blood 117:2319-2331. In some aspects of such embodiments the subject is ECOG 0-1. In aspects, the therapy is indicated for such subjects and/or the instructions indicate administration to a subject within such population. In some embodiments, based on the 2016 WHO criteria (Swerdlow et al., (2016) Blood 127(20):2375-2390), double/triple hit lymphoma can be considered high-grade B-cell lymphoma, with MYC and BCL2 and/or BCL6 rearrangements with DLBCL histology (double/triple hit).

In some embodiments, the combination therapy is administered to subjects who are or are likely to be or who are predicted to be poor responders and/or who do not, are likely not to and/or who are predicted not to respond or do not respond within a certain time and/or to a certain extent to treatment with a cell therapy (e.g. CAR+ T cells). In some embodiments, the combination therapy is administered to subjects who do not or are not likely to or are not predicted to exhibit a complete response or overall response, such as within 1 month, within two months or within three months after initiation of administration of a cell therapy. In some embodiments, the combination therapy is administered to subjects who exhibit or are likely to exhibit or who are predicted to exhibit progressive disease (PD), such as within 1 month, two months or three months, following administration of the cell therapy. In some embodiments, a subject is likely or predicted not to exhibit a response or a certain response based on a plurality of similarly situated subjects so treated or previously treated with the cell therapy.

In some embodiments, the provided methods involve treating a specific group or subset of subjects, e.g., subjects identified as having high-risk disease, e.g., high-risk NHL. In some aspects, the methods treat subjects having a form of aggressive and/or poor prognosis B-cell non-Hodgkin lymphoma (NHL), such as NHL that has relapsed or is refractory (R/R) to standard therapy has a poor prognosis. In some cases, the overall response rate (ORR) to available therapies, to a standard of care, or to a reference therapy for the disease and/or patient population for which the therapy is indicated, is less than 40% and/or the complete response (CR) is less than 20%. In some embodiments, in chemorefractory DLBCL, the ORR with a reference or available treatment or standard-of-care therapy is about 26% and the CR is about 8% (Crump et al. Outcomes in refractory aggressive diffuse large B-cell lymphoma (DLBCL): Results from the international SCHOLAR study. ASCO 2016 [Abstract 7516]). In some aspects, the provided methods, compositions, uses and articles of manufacture achieve improved and superior responses to available therapies.

In some embodiments, the methods and uses for treatment of subjects described herein involves selecting or identifying a particular group or subset of subjects, e.g., based on specific types of disease, diagnostic criteria, prior treatments and/or response to prior treatments. In some embodiments, the methods involve treating a subject having relapsed following remission after treatment with, or become refractory to, one or more prior therapies; or a subject that has relapsed or is refractory (R/R) to one or more prior therapies, e.g., one or more lines of standard therapy including those as described herein.

In some embodiments, the subject has been subject to more than one, two three, four, five, or six prior therapies. In some embodiments, the subject has been subject to one prior therapy. In some embodiments, the subject has been subject to about two to four prior therapies. In some embodiments, the subject has been subject to about five to six prior therapies. In some embodiments, the subject has been subject to more than six prior therapies.

In some embodiments, the subject has been previously treated with a therapy or a therapeutic agent targeting the lymphoma, e.g., NHL, prior to administration of the cells expressing the recombinant receptor. In some embodiments, the subject has been previously treated with a cell therapy (e.g., CAR+ T cells). In some embodiments, the subject has been previously treated with a hematopoietic stem cell transplantation (HSCT), e.g., allogenic HSCT or autogenic HSCT. In some embodiments, the subject has had poor prognosis after treatment with standard therapy and/or has failed one or more lines of previous therapy. In some embodiments, the subject has been treated or has previously received at least or about at least or about 1, 2, 3, 4, 5, 6, or 7 other therapies for treating the NHL other than a lymphodepleting therapy. In some embodiments, the subject has been previously treated with chemotherapy or radiation therapy. In some aspects, the subject is refractory or non-responsive to the other therapy or therapeutic agent. In some embodiments, the subject has persistent or relapsed disease, e.g., following treatment with another therapy or therapeutic intervention, including chemotherapy or radiation.

In some embodiments, the combination therapy is administered to subjects that have progressed on a prior treatment. In some embodiments, the combination therapy is administered to subjects that have stopped responding to a prior therapy. In some embodiments, the combination therapy is administered to subjects that have relapsed following a remission after a prior treatment. In some embodiments, the combination therapy is administered to subjects that are refractory to a prior treatment. In some embodiments, the combination therapy is administered to subjects that have less than an optimal response (e.g., a complete response, a partial response or a stable disease) to a prior therapy.

In some embodiments, the subjects are refractory to last prior therapy. In some embodiments, the subjects have a relapse to last prior therapy. The status is refractory if a subject achieved less than a partial response to last prior therapy. In some embodiments, the subjects have a prior chemotherapy. In some embodiments, the subjects are chemorefractory to the prior chemotherapy. In some embodiments, the subjects are chemosensitive to the prior therapy. The status is chemorefractory is a subject achieved stable disease (SD) or progressive disease (PD) to last chemotherapy-containing regimen or relapsed less than 12 months after autologous stem cell transplant. Otherwise the status is chemosensitive.

In some embodiments, the prior treatment or therapy comprises a CD20-targeted agent. In some embodiments, the prior treatment or therapy comprises an anthracycline. In some embodiments, the prior treatment or therapy comprises a cell therapy (e.g., a T cell therapy, e.g. a CAR T cell therapy).

In some embodiments, the methods, uses and articles of manufacture involve, or are used for treatment of subjects involving, selecting or identifying a particular group or subset of subjects, e.g., based on specific types of disease, diagnostic criteria, prior treatments and/or response to prior treatments, such as any group of subjects as described. In some embodiments, the methods involve treating a subject having relapsed following remission after treatment with, or become refractory to, one or more prior therapies; or a subject that has relapsed or is refractory (R/R) to one or more prior therapies, e.g., one or more lines of standard therapy, e.g., a cell therapy (e.g., CAR+ T cells). In some embodiments, the methods involve treating subjects having diffuse large B-cell lymphoma (DLBCL), not otherwise specified (NOS; de novo and transformed from indolent), primary mediastinal (thymic) large B-cell lymphoma (PMBCL) or follicular lymphoma grade 3B (FL3B), EBV positive DLBCL, or EBV positive NOS. In some embodiments, the methods involve treating a subject that has an Eastern Cooperative Oncology Group Performance Status (ECOG) of less than 1, such as 0-1. In some embodiments, the methods treat a poor-prognosis population or of DLBCL patients or subject thereof that generally responds poorly to therapies or particular reference therapies, such as one having one or more, such as two or three, chromosomal translocations (such as so-called “double-hit” or “triple-hit” lymphoma, which is high grade B-cell lymphoma with MYC and BCL2 and/or BCL6 rearrangements with DLBCL histology; having translocations MYC/8q24 loci, usually in combination with the t (14; 18) (q32; q21) bcl-2 gene or/and BCL6/3q27 chromosomal translocation; see, e.g., Xu et al. (2013) Int J Clin Exp Pathol. 6(4): 788-794), and/or one having relapsed, optionally relapsed within 12 months, and/or one having been deemed chemorefractory.

In some embodiments, the subject has DLBCL that is a germinal center-like (GCB) DLBCL. In some embodiments, the subject has a non-germinal center-like (non-GCB) DLBCL. In some embodiments, the subject has double-hit lymphoma (DHL). In some embodiments, the subject has a triple-hit lymphoma (THL). In some embodiments, the subject is positive for the expression of a gene indicative of the responsiveness of the treatment with Compound C. In some embodiments, the subject is negative for the expression of the gene. See Blood 2017 130:4118.

In some embodiments, the antigen receptor (e.g. CAR) specifically binds to a target antigen associated with the disease or condition, such as associated with NHL. In some embodiments, the antigen associated with the disease or disorder is selected from CD20, CD19, CD22, ROR1, CD45, CD21, CD5, CD33, Igkappa, Iglambda, CD79a, CD79b or CD30. In some embodiments, the antigen is CD19. In some embodiments, the CD19 antigen is a human CD19.

In some embodiments, the methods include administration of the cell therapy and Compound C to a subject, which is, at risk for, or suspected of having a B cell malignancy.

In some embodiments, the methods include administration of cells to a subject selected or identified as having a certain prognosis or risk of NHL. Non-Hodgkin lymphoma (NHL) can be a variable disease. Some subjects with NHL may survive without treatment while others may require immediate intervention. In some cases, subjects with NHL may be classified into groups that may inform disease prognosis and/or recommended treatment strategy. In some cases, these groups may be “low risk,” “intermediate risk,” “high risk,” and/or “very high risk” and patients may be classified as such depending on a number of factors including, but not limited to, genetic abnormalities and/or morphological or physical characteristics. In some embodiments, subjects treated in accord with the methods, and/or with the articles of manufacture or compositions, are classified or identified based on the risk of NHL. In some embodiments, the subject is one that has high risk NHL.

In some embodiments, the subject to be treated includes a group of subjects with aggressive NHL, in particular, with diffuse large B-cell lymphoma (DLBCL), not otherwise specified (NOS; de novo and transformed from indolent), T cell/histiocyte-rich large B-cell lymphoma, primary mediastinal (thymic) large B-cell lymphoma (PMBCL), follicular lymphoma grade 3B (FL3B), EBV positive DLBCL, EBV positive NOS, or high grade B-cell lymphoma with MYC and BCL2 and/or BCL6 rearrangements with DLBCL histology (“double-hit” or “triple-hit” lymphoma). In some embodiments, the subject's disease has relapsed or been refractory to at least two prior lines of therapy. In some embodiments, the prior therapy comprises a CD20-targeted agent and/or an anthracycline. In some embodiments, the subjects have a ECOG score of 0-1 at screening. In some embodiments, the subjects have positron emission tomography (PET)-positive disease as per Lugano Classification (Cheson, 2014). In some embodiments, the subject may optionally have previously been treated with allogenic stem cell transplantation (SCT).

In some embodiments, the subject is an adult. In some embodiments, the subjects are male. In some embodiments, the subjects are female. In some embodiments, the subjects are at least 40 years old at the time they are administered the combination therapy (e.g., at the time they are administered the cell therapy). In some embodiments, the subjects are less than 40 years old at the time they are administered the combination therapy (e.g., at the time they are administered the cell therapy). In some embodiments, the subjects are about 40-65 years old at the time they are administered the combination therapy (e.g., at the time they are administered the cell therapy). In some embodiments, the subjects are at least 65 years old at the time they are administered the combination therapy (e.g., at the time they are administered the cell therapy).

### A. Administration of Cell Therapy

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors. Exemplary engineered cells for administering as a cell therapy in the provided methods are described in Section II.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4 vs CD8 T cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some instances, the subject is administered about 500 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy. In some instances, the subject is administered about 500 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose. In some embodiments, the subject is administered 300 mg/m² of cyclophosphamide and 30 mg/m² of fludarabine both daily for 3 days prior to initiation of the cell therapy. In some embodiments, the subject is administered 500 mg/m² of cyclophosphamide and 30 mg/m² of fludarabine both daily for 3 days prior to initiation of the cell therapy.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

### 1. Compositions and Formulations

In some embodiments, the dose of cells of the cell therapy, such as a T cell therapy comprising cells engineered with a recombinant antigen receptor, e.g. CAR or TCR, is provided as a composition or formulation, such as a pharmaceutical composition or formulation. Such compositions can be used in accord with the provided methods and/or with the provided articles of manufacture or compositions, such as in the treatment of a B cell malignancy.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the cell therapy, such as engineered T cells (e.g. CAR T cells), are formulated with a pharmaceutically acceptable carrier. In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat the disease or condition, such as a therapeutically effective or prophylactically effective amount. Therapeutic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### 2. Dosing

In some embodiments, a dose of cells is administered to subjects in accord with the provided methods, and/or with the provided articles of manufacture or compositions. In some embodiments, the size or timing of the doses is determined as a function of the particular disease or condition (e.g., cancer, e.g., B cell malignancy) in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, the dose of cells is a flat dose of cells or fixed dose of cells such that the dose of cells is not tied to or based on the body surface area or weight of a subject.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸ 2×10⁸, 3×10⁸, or 4×10⁸ total such cells, or the range between any two of the foregoing values. In some embodiments, where the subject is a human, the dose includes between about 1×10⁶ and 3×10⁸ total recombinant receptor (e.g., CAR)-expressing cells, e.g., in the range of about 1×10⁷ to 2×10⁸ such cells, such as 1×10⁷, 5×10⁷, 1×10⁸ or 1.5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁵ to 5×10⁸ total recombinant receptor (e.g. CAR)-expressing T cells or total T cells, 1×10⁵ to 1×10⁸ total recombinant receptor (e.g. CAR)-expressing T cells or total T cells, from or from about 5×10⁵ to 1×10⁷ total recombinant receptor (e.g. CAR)r-expressing T cells or total T cells, or from or from about 1×10⁶ to 1×10⁷ total recombinant receptor (e.g. CAR)-expressing T cells or total T cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 1×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷ or 1×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷ or 1×10⁸ total recombinant receptor-expressing CD8+ T cells.

In some embodiments, for example, where the subject is human, the CD4+ T cells of the dose, including in a dose including CD4+ and CD8+ T cells, includes between about 1×10⁶ and 1×10⁸ total recombinant receptor (e.g., CAR)-expressing CD4+ cells, e.g., in the range of about 5×10⁶ to 1×10⁸ such cells, such cells 1×10⁷, 2.5×10⁷, 5×10⁷, 7.5×10⁷ or 1×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD4+ T cells, 1×10⁷ to 2.5×10⁷ total recombinant receptor-expressing CD4+ T cells, from or from about 1×10⁷ to 0.75×10⁸ total recombinant receptor-expressing CD4+ T cells, each inclusive. In some embodiments, the dose of cells comprises the administration of or about 1×10⁷, 2.5×10⁷, 5×10⁷ 7.5×10⁷ or 1×10⁸ total recombinant receptor-expressing CD4+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some embodiments, the first composition and the second composition is mixed prior to the administration into the subject. In some embodiments, the first composition and the second composition is mixed shortly (e.g., within 6 hours, 5 hours, 4 hours, 3 hours, 2 hours, 1.5 hours, 1 hour, or 0.5 hour) before the administration, In some embodiments, the first composition and the second composition is mixed immediately before the administration.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

### B. Administration of Compound C

In some embodiments of the methods, compositions, combinations, kits or articles of manufacture provided herein, the combination therapy comprises administering Compound C having the chemical name (S)-2-(2,6-dioxopiperidin-3-yl)-4-((2-fluoro-4-((3-morpholinoazetidin-1-yl)methyl)benzyl)amino)isoindoline-1,3-dione and/or that has structure of Formula I:

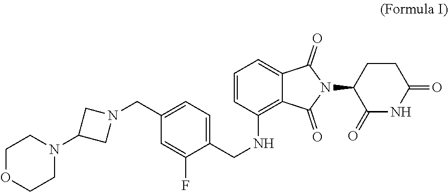 [SMILES: O=C1CC[C@H](N2C(=O)c3cccc(NCc4ccc(CN5CC(N6CCOCC6)C5)cc4F)c3C2=O)C(=O)N1]

or an enantiomer or a mixture of enantiomers thereof, or a pharmaceutically acceptable salt, solvate, hydrate, co-crystal, clathrate, or polymorph thereof.

In some embodiments, Compound C is an enantiomer or a mixture of enantiomers of (S)-2-(2,6-dioxopiperidin-3-yl)-4-((2-fluoro-4-((3-morpholinoazetidin-1-yl)methyl)benzyl)amino)isoindoline-1,3-dione, or a pharmaceutically acceptable salt, solvate, hydrate, co-crystal, clathrate, or polymorph of (S)-2-(2,6-dioxopiperidin-3-yl)-4-((2-fluoro-4-((3-morpholinoazetidin-1-yl)methyl)benzyl)amino)isoindoline-1,3-dione. In some embodiments, Compound C is a solvate of (S)-2-(2,6-dioxopiperidin-3-yl)-4-((2-fluoro-4-((3-morpholinoazetidin-1-yl)methyl)benzyl)amino)isoindoline-1,3-dione. In some embodiments, Compound C is a hydrate of (S)-2-(2,6-dioxopiperidin-3-yl)-4-((2-fluoro-4-((3-morpholinoazetidin-1-yl)methyl)benzyl)amino)isoindoline-1,3-dione. In some embodiments, Compound C is a pharmaceutically acceptable salt of (S)-2-(2,6-dioxopiperidin-3-yl)-4-((2-fluoro-4-((3-morpholinoazetidin-1-yl)methyl)benzyl)amino)isoindoline-1,3-dione. In some embodiments, Compound C is (S)-2-(2,6-dioxopiperidin-3-yl)-4-((2-fluoro-4-((3-morpholinoazetidin-1-yl)methyl)benzyl)amino)isoindoline-1,3-dione. In some embodiments, Compound C has the structure of Formula I.

In certain embodiments, Compound C is a solid. In certain embodiments, Compound C is hydrated. In certain embodiments, Compound C is solvated. In certain embodiments, Compound C is anhydrous. In certain embodiments, Compound C is nonhygroscopic.

In certain embodiments, Compound C is amorphous. In certain embodiments, Compound C is crystalline.

In some embodiments, Compound C is a pharmaceutically acceptable salt of (S)-2-(2,6-dioxopiperidin-3-yl)-4-((2-fluoro-4-((3-morpholinoazetidin-1-yl)methyl)benzyl)amino)isoindoline-1,3-dione. As used herein, the term “pharmaceutically acceptable salt” refers to a salt prepared from a pharmaceutically acceptable non-toxic acid or base including an inorganic acid and base and an organic acid and base. Suitable pharmaceutically acceptable base addition salts of Compound C include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

In certain embodiments, Compound C is a hydrochloride salt of (S)-2-(2,6-dioxopiperidin-3-yl)-4-((2-fluoro-4-((3-morpholinoazetidin-1-yl)methyl)benzyl)amino)isoindoline-1,3-dione, or an enantiomer or a mixture of enantiomers thereof; or a pharmaceutically acceptable solvate, hydrate, co-crystal, clathrate, or polymorph thereof. In certain embodiments, the hydrochloride salt is a solid. In certain embodiments, the hydrochloride salt is anhydrous. In certain embodiments, the hydrochloride salt is nonhygroscopic. In certain embodiments, the hydrochloride salt is amorphous. In certain embodiments, the hydrochloride salt is crystalline.

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. Methods involving administration of any such isomeric forms of Compound C are included within the embodiments provided herein, including administration of mixtures thereof.

In some embodiments, Compound C provided herein contains one chiral center, and can exist as a mixture of enantiomers, e.g., a racemic mixture. This disclosure encompasses the use of stereomerically pure forms of such a compound, as well as the use of mixtures of those forms. For example, mixtures comprising equal or unequal amounts of the enantiomers of Compound C provided herein may be used in methods and compositions disclosed herein. These isomers may be asymmetrically synthesized or resolved using standard techniques such as chiral columns or chiral resolving agents. See, e.g., Jacques, J., et al, Enantiomers, Racemates and Resolutions (Wiley-Interscience, New York, 1981); Wilen, S. H., et al, Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E L. Eliel, Ed., Univ. of Notre Dame Press, Notre Dame, I N, 1972).

It is to be understood that the chiral centers of Compound C may undergo epimerization in vivo. As such, one of skill in the art will recognize that in the case of epimerization in vivo, administration of Compound C in its (R) form may be equivalent to administration of Compound C in its (S) form.

Optically active (+) and (−), (R)- and (S)-, or (D)- and (L)-isomers may be prepared using chiral synthons or chiral reagents, or resolved using conventional techniques, such as chromatography on a chiral stationary phase.

In some embodiments, Compound C is a solvate of (S)-2-(2,6-dioxopiperidin-3-yl)-4-((2-fluoro-4-((3-morpholinoazetidin-1-yl)methyl)benzyl)amino)isoindoline-1,3-dione. The term “solvate” means a physical association of a compound with one or more solvent molecules, whether organic or inorganic. This physical association includes hydrogen bonding. In certain instances, the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. In some embodiments, Compound C is a hydrate of (S)-2-(2,6-dioxopiperidin-3-yl)-4-((2-fluoro-4-((3-morpholinoazetidin-1-yl)methyl)benzyl)amino)isoindoline-1,3-dione. Methods of solvation are known in the art.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

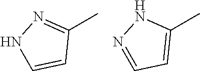

As readily understood by one skilled in the art, a wide variety of functional groups and other structures may exhibit tautomerism, and administration of any tautomer of Compound C is within the scope of the methods provided herein.

It should also be noted that Compound C as used in the provided methods can contain unnatural proportions of atomic isotopes at one or more of the atoms. For example, Compound C may be radiolabeled with radioactive isotopes, such as for example tritium (3H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g, in vivo imaging agents. Methods involving administration of any isotopic variation of Compound C, whether radioactive or not, are intended to be encompassed within the scope of the methods provided herein. In some embodiments, provided herein are methods involving administration of isotopologues of Compound C, for example deuterium, carbon-13 (¹³C), and/or nitrogen-15 (¹⁵N) enriched compounds. As used herein, “deuterated” means a compound wherein at least one hydrogen (H) has been replaced by deuterium (indicated by D or 2H), that is, the compound is enriched in deuterium in at least one position.

It is understood that, independently of stereomerical or isotopic composition, Compound C can be administered in the form of any of the pharmaceutically acceptable salts described herein. Equally, it is understood that the isotopic composition may vary independently from the stereomerical composition of Compound C. Further, the isotopic composition, while being restricted to those elements present in Compound C or salt thereof, may otherwise vary independently from the selection of the pharmaceutically acceptable salt of Compound C.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

### 1. Compositions and Formulations

In some embodiments of the combination therapy methods, compositions, combinations, kits and uses provided herein, the combination therapy can be administered in one or more compositions, e.g., a pharmaceutical composition containing Compound C.

In some embodiments, the composition, e.g., a pharmaceutical composition containing Compound C can include carriers such as a diluent, adjuvant, excipient, or vehicle with which Compound C and/or the cells are administered. Examples of suitable pharmaceutical carriers are described in “Remington's Pharmaceutical Sciences” by E. W. Martin. Such compositions will contain a therapeutically effective amount of Compound C, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular inhibitor and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. The compositions containing Compound C can also be lyophilized.

In some embodiments, the pharmaceutical compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration. In some embodiments, it is administered by multiple bolus administrations, for example, over a period of no more than 3 days, or by continuous infusion administration.

In some embodiments, the administration can be local, topical or systemic depending upon the locus of treatment. In some embodiments local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. In some embodiments, compositions also can be administered with other biologically active agents, either sequentially, intermittently or in the same composition. In some embodiments, administration also can include controlled release systems including controlled release formulations and device controlled release, such as by means of a pump. In some embodiments, the administration is oral.

In some embodiments, Compound C are typically formulated and administered in unit dosage forms or multiple dosage forms. Each unit dose contains a predetermined quantity of therapeutically active Compound C sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of Compound C. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

### 2. Dosing

In some embodiments, the provided combination therapy methods involve initiating administration of Compound C prior to, subsequently to, during, during the course of, simultaneously, near simultaneously, sequentially, concurrently and/or intermittently with the initiation of the cell therapy, such as a T cell therapy (e.g., CAR-expressing T cells). In some embodiments, initiation of administration of Compound C in the provided combination therapy methods is after or subsequent to initiation of administration of the T cell therapy.

In some embodiments, the administration of Compound C is initiated after (subsequent to) the initiation of the cell therapy, such as a T cell therapy (e.g., CAR-expressing T cells). In some embodiments, administration of Compound C is initiated at or before peak or maximum level of the cells of the T cell therapy is detectable in the blood of the subject.

In some cases, initiation of administration Compound C is carried out at or within a week, such as within 1, 2 or 3 days before (i) a time in which peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (iv) the subject exhibits increased tumor burden as compared to tumor burden at a time prior to or after administration of the cells and prior to initiation of administration of Compound C. In certain aspects, the provided methods are carried out to enhance, increase or potentiate T cell therapy in subjects to improve response to the T cell therapy, e.g. presence of T cells and/or reduction in tumor burden.

In some embodiments, the cell therapy, such as a T cell therapy (e.g., CAR-expressing T cells), is administered on Day 1 of the combination therapy.

In some embodiments, the administration of Compound C is initiated after (subsequent to) the initiation of the cell therapy. In some embodiments, the administration of Compound C is initiated after (subsequent to) the initiation of the T cell therapy and as early as the same day of the initiation of the T cell therapy (i.e., as early as Day 1 of the combination therapy). In some embodiments, the administration of Compound C is initiated between Day 1 and Day 29, inclusive, of the combination therapy. In some embodiments, the administration of Compound C is initiated between Day 1 and Day 22, inclusive, of the combination therapy. In some embodiments, the administration of Compound C is initiated between Day 1 and Day 15, inclusive, of the combination therapy. In some embodiments, the administration of Compound C is initiated between Day 2 and Day 15, inclusive, of the combination therapy. In some embodiments, the administration of Compound C is initiated between Day 3 and Day 15, inclusive, of the combination therapy. In some embodiments, the administration of Compound C is initiated between Day 4 and Day 15, inclusive, of the combination therapy. In some embodiments, the administration of Compound C is initiated between Day 5 and Day 15, inclusive, of the combination therapy. In some embodiments, the administration of Compound C is initiated between Day 6 and Day 15, inclusive, of the combination therapy. In some embodiments, the administration of Compound C is initiated between Day 7 and Day 15, inclusive, of the combination therapy. In some embodiments, the administration of Compound C is initiated between Day 8 and Day 15, inclusive, of the combination therapy.

In some embodiments, the administration of Compound C is initiated at or about Day 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the combination therapy. In some embodiments, the administration of Compound Cis initiated at or about Day 1 of the combination therapy. In some embodiments, the administration of Compound C is initiated at or about Day 2 of the combination therapy. In some embodiments, the administration of Compound C is initiated at or about Day 3 of the combination therapy. In some embodiments, the administration of Compound C is initiated at or about Day 4 of the combination therapy. In some embodiments, the administration of Compound C is initiated at or about Day 5 of the combination therapy. In some embodiments, the administration of Compound C is initiated at or about Day 5 of the combination therapy. In some embodiments, the administration of Compound C is initiated at or about Day 7 of the combination therapy. In some embodiments, the administration of Compound C is initiated at or about Day 8 of the combination therapy. In some embodiments, the administration of Compound C is initiated at or about Day 9 of the combination therapy. In some embodiments, the administration of Compound C is initiated at or about Day 10 of the combination therapy. In some embodiments, the administration of Compound C is initiated at or about Day 11 of the combination therapy. In some embodiments, the administration of Compound C is initiated at or about Day 12 of the combination therapy. In some embodiments, the administration of Compound C is initiated at or about Day 13 of the combination therapy. In some embodiments, the administration of Compound C is initiated at or about Day 14 of the combination therapy. In some embodiments, the administration of Compound C is initiated at or about Day 15 of the combination therapy. In some embodiments, the administration of Compound C is initiated at or about Day 16 of the combination therapy. In some embodiments, the administration of Compound C is initiated at or about Day 17 of the combination therapy. In some embodiments, the administration of Compound C is initiated at or about Day 18 of the combination therapy. In some embodiments, the administration of Compound C is initiated at or about Day 19 of the combination therapy. In some embodiments, the administration of Compound C is initiated at or about Day 20 of the combination therapy. In some embodiments, the administration of Compound C is initiated at or about Day 21 of the combination therapy. In some embodiments, the administration of Compound C is initiated at or about Day 22 of the combination therapy.

In some embodiments, the administration of Compound C is initiated prior to the administration of the cell therapy, e.g., T cell therapy. In some embodiments, the administration of Compound C is initiated within one month prior to the administration of the cell therapy. In some embodiments, the administration of Compound C is initiated within four weeks prior to the administration of the cell therapy. In some embodiments, the administration of Compound C is initiated within three weeks prior to the administration of the cell therapy. In some embodiments, the administration of Compound C is initiated within two weeks prior to the administration of the cell therapy. In some embodiments, the administration of Compound C is initiated within one week prior to the administration of the cell therapy. In some embodiments, the administration of Compound C is initiated within six days prior to the administration of the cell therapy. In some embodiments, the administration of Compound C is initiated within five days prior to the administration of the cell therapy. In some embodiments, the administration of Compound C is initiated within four days prior to the administration of the cell therapy. In some embodiments, the administration of Compound C is initiated within three days prior to the administration of the cell therapy. In some embodiments, the administration of Compound C is initiated within two days prior to the administration of the cell therapy. In some embodiments, the administration of Compound C is initiated within one day prior to the administration of the cell therapy.

In some embodiments, the administration of Compound C is initiated one month prior to the administration of the cell therapy. In some embodiments, the administration of Compound C is initiated four weeks prior to the administration of the cell therapy. In some embodiments, the administration of Compound C is initiated three weeks prior to the administration of the cell therapy. In some embodiments, the administration of Compound C is initiated two weeks prior to the administration of the cell therapy. In some embodiments, the administration of Compound C is initiated one week prior to the administration of the cell therapy. In some embodiments, the administration of Compound C is initiated six days prior to the administration of the cell therapy. In some embodiments, the administration of Compound C is initiated five days prior to the administration of the cell therapy. In some embodiments, the administration of Compound C is initiated four days prior to the administration of the cell therapy. In some embodiments, the administration of Compound C is initiated three days prior to the administration of the cell therapy. In some embodiments, the administration of Compound C is initiated two days prior to the administration of the cell therapy. In some embodiments, the administration of Compound C is initiated one day prior to the administration of the cell therapy.

In some embodiments, the administration of Compound C is initiated following the administration of the lymphodepleting therapy and prior to the administration of the cell therapy. In some embodiments, the cell therapy is administered two to seven days following the end of the administration of the lymphodepleting therapy. In some embodiments, the administration of Compound C is initiated on the last day of the lymphodepleting therapy. In some embodiments, the administration of Compound C is initiated within seven days following the end of the administration of the lymphodepleting therapy. In some embodiments, the administration of Compound C is initiated within six days following the end of the administration of the lymphodepleting therapy. In some embodiments, the administration of Compound C is initiated within five days following the end of the administration of the lymphodepleting therapy. In some embodiments, the administration of Compound C is initiated within four days following the end of the administration of the lymphodepleting therapy. In some embodiments, the administration of Compound C is initiated within three days following the end of the administration of the lymphodepleting therapy. In some embodiments, the administration of Compound C is initiated within two days following the end of the administration of the lymphodepleting therapy. In some embodiments, the administration of Compound C is initiated within one day following the end of the administration of the lymphodepleting therapy.

In some embodiments, the administration of Compound C is initiated seven days following the end of the administration of the lymphodepleting therapy. In some embodiments, the administration of Compound C is initiated six days following the end of the administration of the lymphodepleting therapy. In some embodiments, the administration of Compound C is initiated five days following the end of the administration of the lymphodepleting therapy. In some embodiments, the administration of Compound C is initiated four days following the end of the administration of the lymphodepleting therapy. In some embodiments, the administration of Compound C is initiated three days following the end of the administration of the lymphodepleting therapy. In some embodiments, the administration of Compound C is initiated two days following the end of the administration of the lymphodepleting therapy. In some embodiments, the administration of Compound C is initiated one day following the end of the administration of the lymphodepleting therapy.

In some embodiments, at the time at which the subject is first administered Compound C and/or at any subsequent time after initiation of the administration, the subject does not exhibit a sign or symptom of a severe toxicity, such as severe cytokine release syndrome (CRS) or severe toxicity. In some embodiments, the administration of Compound C is at a time at which the subject does not exhibit a sign or symptom of severe CRS and/or does not exhibit grade 3 or higher CRS, such as prolonged grade 3 CRS or grade 4 or 5 CRS. In some embodiments, the administration of Compound C is at a time at which the subject does not exhibit a sign or symptom of severe neurotoxicity and/or does not exhibit grade 3 or higher neurotoxicity, such as prolonged grade 3 neurotoxicity or grade 4 or grade 5 neurotoxicity. In some aspects, between the time of the initiation of the administration of the T cell therapy and the time of the administration of Compound C, the subject has not exhibited severe CRS and/or has not exhibited grade 3 or higher CRS, such as prolonged grade 3 CRS or grade 4 or 5 CRS. In some instances, between the time of the initiation of the administration of the T cell therapy and the time of the administration of Compound C, the subject has not exhibited severe neurotoxicity and/or does not exhibit grade 3 or higher neurotoxicity, such as prolonged grade 3 neurotoxicity or grade 4 or 5 neurotoxicity.

In some embodiments, Compound C is administered in an intermittent (i.e., non-daily) dosing regimen. In some embodiments, Compound C is administered as a plurality of intermittent doses. In some embodiments, each dose of the plurality of intermittent doses of Compound C is the same. In other embodiments, the plurality of intermittent doses of Compound C can be of different amounts.

In some embodiments, the dose of Compound C is between at or about 0.1 mg and at or about 1.0 mg, such as between at or about 0.1 mg and 0.9 mg, between at or about 0.1 mg and 0.8 mg, between at or about 0.1 mg and 0.7 mg, between at or about 0.1 mg and 0.6 mg, between at or about 0.1 mg and 0.5 mg, between at or about 0.1 mg and 0.4 mg, between at or about 0.1 mg and 0.3 mg, between at or about 0.1 mg and 0.2 mg, between at or about 0.2 mg and 1.0 mg, between at or about 0.2 mg and 0.9 mg, between at or about 0.2 mg and 0.8 mg, between at or about 0.2 mg and 0.7 mg, between at or about 0.2 mg and 0.6 mg, between at or about 0.2 mg and 0.5 mg, between at or about 0.2 mg and 0.4 mg, between at or about 0.2 mg and 0.3 mg, between at or about 0.3 mg and 1.0 mg, between at or about 0.3 mg and 0.9 mg, between at or about 0.3 mg and 0.8 mg, between at or about 0.3 mg and 0.7 mg, between at or about 0.3 mg and 0.6 mg, between at or about 0.3 mg and 0.5 mg, between at or about 0.3 mg and 0.4 mg, between at or about 0.4 mg and 1.0 mg, between at or about 0.4 mg and 0.9 mg, between at or about 0.4 mg and 0.8 mg, between at or about 0.4 mg and 0.7 mg, between at or about 0.4 mg and 0.6 mg, between at or about 0.4 mg and 0.5 mg, between at or about 0.5 mg and 1.0 mg, between at or about 0.5 mg and 0.9 mg, between at or about 0.5 mg and 0.8 mg, between at or about 0.5 mg and 0.7 mg, between at or about 0.5 mg and 0.6 mg, between at or about 0.6 mg and 1.0 mg, between at or about 0.6 mg and 0.9 mg, between at or about 0.6 mg and 0.8 mg, between at or about 0.6 mg and 0.7 mg, between at or about 0.7 mg and 1.0 mg, between at or about 0.7 mg and 0.9 mg, between at or about 0.7 mg and 0.8 mg, between at or about 0.8 mg and 1.0 mg, between at or about 0.8 mg and 0.9 mg, or between at or about 0.8 mg and 1.0 mg, each inclusive. In some embodiments, the dose of Compound C is between at or about 0.1 mg and at or about 0.6 mg. In some embodiments, the dose of Compound C is between at or about 0.1 mg and at or about 0.5 mg. In some embodiments, the dose of Compound C is between at or about 0.1 mg and at or about 0.4 mg. In some embodiments, the dose of Compound C is between at or about 0.1 mg and at or about 0.3 mg. In some embodiments, the dose of Compound C is at or about 0.1 mg. In some embodiments, the dose of Compound C is at or about 0.2 mg. In some embodiments, the dose of Compound C is at or about 0.3 mg. In some embodiments, the dose of Compound C is at or about 0.4 mg. In some embodiments, the dose of Compound C is at or about 0.5 mg. In some embodiments, the dose of Compound C is at or about 0.6 mg. In any of the preceding embodiments, each dose of the plurality of intermittent doses of Compound C is the same.

In some embodiments, Compound C as part of an intermittent dosing regimen is administered no more than once every five days. In some embodiments, Compound C is administered no more than once every six days. In some embodiments, Compound C is administered no more than once every seven days. In some embodiments, Compound C is administered no more than once every eight days. In some embodiments, Compound C is administered no more than once every nine days. In some embodiments, Compound C is administered no more than once every 10 days. In some embodiments, Compound C is administered no more than once every 11 days. In some embodiments, Compound C is administered no more than once every 12 days. In some embodiments, Compound C is administered no more than once every 13 days. In some embodiments, Compound C is administered no more than once every 14 days.

In some embodiments, Compound C as part of an intermittent dosing regimen is administered once every five days. In some embodiments, Compound C is administered once every six days. In some embodiments, Compound C is administered once weekly. In some embodiments, Compound C is administered once every seven days (Q7D). In some embodiments, Compound C is administered once every eight days. In some embodiments, Compound C is administered once every nine days. In some embodiments, Compound C is administered once every 10 days. In some embodiments, Compound C is administered once every 11 days. In some embodiments, Compound C is administered once every 12 days. In some embodiments, Compound C is administered once every 13 days. In some embodiments, Compound C is administered once every two weeks. In some embodiments, Compound C is administered once every 14 days (Q14D).

In some embodiments, Compound C is administered for a period of time subsequent to initiation of administration of the cell therapy. In some embodiments, Compound C is administered for at least one week after initiation of administration of the cell therapy. In some embodiments, Compound C is administered for at least two weeks after initiation of administration of the cell therapy. In some embodiments, Compound C is administered for at least three weeks after initiation of administration of the cell therapy. In some embodiments, Compound C is administered for at least four weeks after initiation of administration of the cell therapy. In some embodiments, Compound C is administered for at least five weeks after initiation of administration of the cell therapy. In some embodiments, Compound C is administered for at least six weeks after initiation of administration of the cell therapy. In some embodiments, Compound C is administered for at least seven weeks after initiation of administration of the cell therapy. In some embodiments, Compound C is administered for at least eight weeks after initiation of administration of the cell therapy. In some embodiments, Compound C is administered for at least nine weeks after initiation of administration of the cell therapy. In some embodiments, Compound C is administered for at least 10 weeks after initiation of administration of the cell therapy. In some embodiments, Compound C is administered for at least 11 weeks after initiation of administration of the cell therapy. In some embodiments, Compound C is administered for at least 12 weeks after initiation of administration of the cell therapy. In some embodiments, Compound C is administered for up to 12 weeks after initiation of administration of the cell therapy. In any of the preceding embodiments, Compound C is administered for up to 12 weeks after initiation of administration of the cell therapy. Thus, with reference to a combination therapy in which the cell therapy is administered on Day 1 of the combination therapy, in some embodiments Compound C is not administered later than Day 85 of the combination therapy.

In some embodiments, Compound C is administered on Days 1, 8, 15, 22, 29, 36, 43, 50, 57, 64, 71, 78, and 85 of the combination therapy. In some embodiments, Compound C is administered on Days 8, 15, 22, 29, 36, 43, 50, 57, 64, 71, 78, and 85 of the combination therapy. In some embodiments, Compound C is administered on Days 15, 22, 29, 36, 43, 50, 57, 64, 71, 78, and 85 of the combination therapy. In some embodiments, Compound C is administered on Days 8, 22, 36, 50, 64, and 78 of the combination therapy.

In some embodiments, each dose of the plurality of intermittent doses of Compound C is 0.3 mg, and Compound C is administered on Days 8, 15, 22, 29, 36, 43, 50, 57, 64, 71, 78, and 85.

In some embodiments, each dose of the plurality of intermittent doses of Compound C is 0.3 mg, and Compound C is administered on Days 1, 8, 15, 22, 29, 36, 43, 50, 57, 64, 71, 78, and 85.

In some embodiments, each dose of the plurality of intermittent doses of Compound C is 0.3 mg, and Compound C is administered on Days 15, 22, 29, 36, 43, 50, 57, 64, 71, 78, and 85.

In some embodiments, each dose of the plurality of intermittent doses of Compound C is 0.4 mg, and Compound C is administered on Days 8, 15, 22, 29, 36, 43, 50, 57, 64, 71, 78, and 85.

In some embodiments, each dose of the plurality of intermittent doses of Compound C is 0.3 mg, and Compound C is administered on Days 8, 22, 36, 50, 64, and 78.

In some embodiments, each dose of the plurality of intermittent doses of Compound C is 0.2 mg, and Compound C is administered on Days 8, 15, 22, 29, 36, 43, 50, 57, 64, 71, 78, and 85.

In some embodiments, each dose of the plurality of intermittent doses of Compound C is 0.6 mg, and Compound C is administered on Days 8, 15, 22, 29, 36, 43, 50, 57, 64, 71, 78, and 85.

In some cases, the cycling regimen can be interrupted at any time, and/or for one or more times. In some cases, the cycling regimen is interrupted or modified if the subject develops one or more adverse event, dose-limiting toxicity (DLT), neutropenia or febrile neutropenia, thrombocytopenia, cytokine release syndrome (CRS) and/or neurotoxicity (NT), such as those as described in Section IV. In some embodiments, the amount of Compound C for each administration or per day in certain days of a week is altered after the subject develops one or more adverse event, dose-limiting toxicity (DLT), neutropenia or febrile neutropenia, thrombocytopenia, cytokine release syndrome (CRS) and/or neurotoxicity (NT), such as those as described in Section IV.

### II. CELL THERAPY AND ENGINEERING CELLS

In some embodiments, the cell therapy (e.g., T cell therapy) for use in accord with the provided combination therapy methods includes administering engineered cells expressing recombinant receptors designed to recognize and/or specifically bind to antigens associated with the disease or condition, such as a cancer, e.g., B cell malignancy. In some embodiments, binding to the antigen results in a response, such as an immune response against such antigens. In some embodiments, the cells contain or are engineered to contain an engineered receptor or recombinant receptor, e.g., an engineered antigen receptor, such as a chimeric antigen receptor (CAR). The recombinant receptor, such as a CAR, generally includes an extracellular antigen (or ligand) binding domain linked to one or more intracellular signaling components, in some aspects via linkers and/or transmembrane domain(s). In some aspects, the engineered cells are provided as pharmaceutical compositions and formulations suitable for administration to a subjects, such as for adoptive cell therapy. Also provided are therapeutic methods for administering the cells and compositions to subjects, e.g., patients. In some embodiments, the methods are any as described in Section I.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

### A. Chimeric Antigen Receptors

In some embodiments of the provided methods and uses, e.g., any as described in Section I, the engineered cells, such as T cells, express a chimeric receptors, such as a chimeric antigen receptors (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446, 191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

Exemplary CAR T cell therapies that target CD19 include those investigated or being investigated in clinical trials NCT02644655, NCT03744676, NCT01087294, NCT03366350, NCT03790891, NCT03497533, NCT04007029, NCT03960840, NCT04049383, NCT04094766, NCT03366324, NCT02546739, NCT03448393, NCT03467256, NCT03488160, NCT04012879, NCT03016377, NCT03468153, NCT03483688, NCT03398967, NCT03229876, NCT03455972, NCT03423706, NCT03497533, and NCT04002401, including FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel). Exemplary engineered cells include BREYANZI®, TECARTUS™, KYMRIAH™, YESCARTA™, UCART19, and ALLO-501. In some aspects, the engineered cells include any of those described in Marofi et al., Front. Immunol. (2021) 12:681984, which is incorporated by reference herein in its entirety.

In some embodiments, the engineered cells, such as T cells, express a recombinant receptor such as a chimeric antigen receptor (CAR) with specificity for a particular antigen (or marker or ligand), such as an antigen expressed on the surface of a particular cell type. In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30. In particular aspects, the antigen is CD19. In some embodiments, any of such antigens are antigens expressed on human B cells.

The chimeric receptors, such as CARs, generally include an extracellular antigen binding domain that is an antigen-binding portion or portions of an antibody molecule. In some embodiments, the antigen-binding domain is a portion of an antibody molecule, generally a variable heavy (VH) chain region and/or variable light (VL) chain region of the antibody, e.g., an scFv antibody fragment. In some embodiments, the antigen-binding domain is a single domain antibody (sdAb), such as sdFv, nanobody, V_{H}H and V_{NAR}. In some embodiments, an antigen-binding fragment comprises antibody variable regions joined by a flexible linker.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19.

In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments the antigen-binding domain includes a V_{H} and/or V_{L} derived from FMC63, which, in some aspects, can be an scFv. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NO: 38 and 39, respectively, and CDR-H3 set forth in SEQ ID NO: 40 or 54 and CDR-L1 set forth in SEQ ID NO: 35 and CDR-L2 set forth in SEQ ID NO: 36 or 55 and CDR-L3 sequences set forth in SEQ ID NO: 37 or 56. In some embodiments, the FMC63 antibody comprises the heavy chain variable region (V_{H}) comprising the amino acid sequence of SEQ ID NO: 41 and the light chain variable region (V_{L}) comprising the amino acid sequence of SEQ ID NO: 42.

In some embodiments, the scFv comprises a variable light chain containing the CDR-LI sequence of SEQ ID NO:35, a CDR-L2 sequence of SEQ ID NO:36, and a CDR-L3 sequence of SEQ ID NO:37 and/or a variable heavy chain containing a CDR-H1 sequence of SEQ ID NO:38, a CDR-H2 sequence of SEQ ID NO:39, and a CDR-H3 sequence of SEQ ID NO:40, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of FMC63 set forth in SEQ ID NO:41 and a variable light chain region of FMC63 set forth in SEQ ID NO:42, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:59. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:57 or a sequence that exhibits at least 85%, 86%, 87%, 88%, 89%, 90%, 91%, 92%, 93%, 94%, 95%, 96%, 97%, 98%, or 99% sequence identity to SEQ ID NO:57. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43. In some embodiments, the anti-CD19 CAR is that of BREYANZI® (lisocabtagene maraleucel). In some embodiments, the T cell therapy is BREYANZI® (lisocabtagene maraleucel).

In some embodiments the antigen-binding domain includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 47-49, respectively, and CDR-L1, CDR-L2 and CDR-L3 sequences set forth in SEQ ID NOS: 44-46, respectively. In some embodiments, the SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51. In some embodiments, the scFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:47, a CDR-H2 sequence of SEQ ID NO:48, and a CDR-H3 sequence of SEQ ID NO:49, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of SJ25C1 set forth in SEQ ID NO:50 and a variable light chain region of SJ25C1 set forth in SEQ ID NO:51, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:52. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

In some embodiments, the anti-CD19 CAR is that of TECARTUS™ (brexucabtagene autoleucel). In some embodiments, the T cell therapy is TECARTUS™ (brexucabtagene autoleucel).

In some embodiments, the anti-CD19 CAR is that of KYMRIAH™ (tisagenlecleucel). In some embodiments, the T cell therapy is KYMRIAH™ (tisagenlecleucel).

In some embodiments, the anti-CD19 CAR is that of YESCARTA™ (axicabtagene ciloleucel). In some embodiments, the T cell therapy is YESCARTA™ (axicabtagene ciloleucel).

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody, V_{H}H or V_{NAR}) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. In some aspects, the CAR is a bispecific CAR, e.g., containing two antigen-binding domains with different specificities.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)₂, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

The terms “complementarity determining region,” and “CDR,” synonymous with “hypervariable region” or “HVR,” are known, in some cases, to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known, in some cases, to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 2, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-LI located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 2
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26-- | H26--H35B | H30--H35B |
| (Kabat | | H32 . . . 34 | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAb) are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include sdFv, nanobody, V_{H}H or V_{NAR}.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some aspects, the recombinant receptor, e.g., a chimeric antigen receptor, includes an extracellular portion containing one or more ligand- (e.g., antigen-) binding domains, such as an antibody or fragment thereof, and one or more intracellular signaling region or domain (also interchangeably called a cytoplasmic signaling domain or region). In some aspects, the recombinant receptor, e.g., CAR, further includes a spacer and/or a transmembrane domain or portion. In some aspects, the spacer and/or transmembrane domain can link the extracellular portion containing the ligand- (e.g., antigen-) binding domain and the intracellular signaling region(s) or domain(s)

In some embodiments, the recombinant receptor such as the CAR, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, Hudecek et al. (2015) Cancer Immunol Res. 3(2): 125-135 or international patent application publication number WO2014031687.

In some embodiments, the spacer contains only a hinge region of an IgG, such as only a hinge of IgG4 or IgG1, such as the hinge only spacer set forth in SEQ ID NO: 1, and encoded by the sequence set forth in SEQ ID NO: 2. In some embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 3. In some embodiments, the spacer the spacer is an Ig hinge, e.g., an IgG4 hinge, linked to a C_{H}3 domain only, such as set forth in SEQ ID NO: 4. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some aspects, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5, 27-34 or 58, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 (4-1BB), or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28 or a variant thereof. The extracellular domain and transmembrane can be linked directly or indirectly. In some embodiments, the extracellular domain and transmembrane are linked by a spacer, such as any described herein.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8. In some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the recombinant receptor, e.g., CAR, includes at least one intracellular signaling component or components, such as an intracellular signaling region or domain. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components. Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the intracellular signaling regions, e.g., comprising intracellular domain or domains, include the cytoplasmic sequences of a region or domain that is involved in providing costimulatory signal.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8alpha, CD8beta, CD4, CD25, or CD16. For example, in some aspects, the CAR or other chimeric receptor includes a chimeric molecule between CD3-zeta (CD3-ζ) or Fc receptor γ and CD8alpha, CD8beta, CD4, CD25 or CD16.

In some embodiments, the intracellular (or cytoplasmic) signaling region comprises a human CD3 chain, optionally a CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40 (CD134), CD27, DAP10, DAP12, ICOS and/or other costimulatory receptors. In some embodiments, the CAR includes a costimulatory region or domain of CD28 or 4-1BB, such as of human CD28 or human 4-1BB.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some aspects, the same CAR includes both the primary (or activating) cytoplasmic signaling regions and costimulatory signaling components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16. An exemplary T2A linker sequence comprises the sequence of amino acids set forth in SEQ ID NO: 6 or 17 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 17.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the chimeric antigen receptor includes an extracellular portion containing the antibody or fragment described herein. In some aspects, the chimeric antigen receptor includes an extracellular portion containing the antibody or fragment described herein and an intracellular signaling domain. In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD3ζ) chain. In some embodiments, the CD3-zeta chain is a human CD3-zeta chain. In some embodiments, the intracellular signaling region further comprises a CD28 and CD137 (4-1BB, TNFRSF9) co-stimulatory domains, linked to a CD3 zeta intracellular domain. In some embodiments, the CD28 is a human CD28. In some embodiments, the 4-1BB is a human 4-1BB. In some embodiments, the chimeric antigen receptor includes a transmembrane domain disposed between the extracellular domain and the intracellular signaling region. In some aspects, the transmembrane domain contains a transmembrane portion of CD28. The extracellular domain and transmembrane can be linked directly or indirectly. In some embodiments, the extracellular domain and transmembrane are linked by a spacer, such as any described herein.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. For example, in some embodiments, the CAR includes an antibody such as an antibody fragment, including scFvs, e.g. specific for CD19 such as any described above, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain.

In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer. In some embodiments, the CAR includes an antibody or fragment, such as scFv, e.g. specific for CD19 such as any described above, a spacer such as any of the Ig-hinge containing spacers, a CD28-derived transmembrane domain, a 4-1BB-derived intracellular signaling domain, and a CD3 zeta-derived signaling domain.

### B. Nucleic Acids, Vectors and Methods for Genetic Engineering

In some embodiments, the cells, e.g., T cells, are genetically engineered to express a recombinant receptor. In some embodiments, the engineering is carried out by introducing polynucleotides that encode the recombinant receptor. Also provided are polynucleotides encoding a recombinant receptor, and vectors or constructs containing such nucleic acids and/or polynucleotides.

In some cases, the nucleic acid sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO:25 and encoded by the nucleotide sequence set forth in SEQ ID NO:24. In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 25 and encoded by the nucleotide sequence set forth in SEQ ID NO:24, or the CD8 alpha signal peptide set forth in SEQ ID NO:26.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor.

In certain cases where nucleic acid molecules encode two or more different polypeptide chains, e.g., a recombinant receptor and a marker, each of the polypeptide chains can be encoded by a separate nucleic acid molecule. For example, two separate nucleic acids are provided, and each can be individually transferred or introduced into the cell for expression in the cell. In some embodiments, the nucleic acid encoding the recombinant receptor and the nucleic acid encoding the marker are operably linked to the same promoter and are optionally separated by an internal ribosome entry site (IRES), or a nucleic acid encoding a self-cleaving peptide or a peptide that causes ribosome skipping, which optionally is a T2A, a P2A, an E2A or an F2A. In some embodiments, the nucleic acids encoding the marker and the nucleic acid encoding the recombinant receptor are operably linked to two different promoters. In some embodiments, the nucleic acid encoding the marker and the nucleic acid encoding the recombinant receptor are present or inserted at different locations within the genome of the cell. In some embodiments, the polynucleotide encoding the recombinant receptor is introduced into a composition containing cultured cells, such as by retroviral transduction, transfection, or transformation.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products ((e.g. encoding the marker and encoding the recombinant receptor) by a message from a single promoter. Alternatively, in some cases, a single promoter may direct expression of an RNA that contains, in a single open reading frame (ORF), two or three genes (e.g. encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No. 20070116690.

Any of the recombinant receptors described herein can be encoded by polynucleotides containing one or more nucleic acid sequences encoding recombinant receptors, in any combinations or arrangements. For example, one, two, three or more polynucleotides can encode one, two, three or more different polypeptides, e.g., recombinant receptors. In some embodiments, one vector or construct contains a nucleic acid sequence encoding marker, and a separate vector or construct contains a nucleic acid sequence encoding a recombinant receptor, e.g., CAR. In some embodiments, the nucleic acid encoding the marker and the nucleic acid encoding the recombinant receptor are operably linked to two different promoters. In some embodiments, the nucleic acid encoding the recombinant receptor is present downstream of the nucleic acid encoding the marker.

In some embodiments, the vector backbone contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16.

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy, 2014 Apr. 3. doi: 10.1038/gt.2014.25; Carlens et al. (2000) Exp. Hematol., 28(10): 1137-46; Alonso-Camino et al. (2013) Mol. Ther. Nucl. Acids., 2, e93; Park et al., Trends Biotechnol., 2011 Nov. 29(11): 550-557.

In some embodiments, the vector is an adeno-associated virus (AAV).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### C. Cells and Preparation of Cells for Genetic Engineering

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood, 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁺ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or subpopulation, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9): 689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° C. per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating or stimulating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### III. EXEMPLARY TREATMENT OUTCOMES AND METHODS FOR ASSESSING SAME

In some embodiments of the methods, compositions, combinations, uses, kits and articles of manufacture provided herein, the provided combination therapy results in one or more treatment outcomes, such as a feature associated with any one or more of the parameters associated with the therapy or treatment, as described below. In some embodiments, the method is any as described in Section I. In some embodiments, the method further includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells, e.g. T cell therapy, before, during, or after administering the combination therapy provided herein.

In some embodiments, the combination therapy can further include one or more screening steps to identify subjects for treatment with the combination therapy and/or continuing the combination therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the combination therapy provided herein.

In some embodiments, any of the screening steps and/or assessment of treatment of outcomes described herein can be used prior to, during, during the course of, or subsequent to administration of one or more steps of the provided combination therapy, e.g., administration of the T cell therapy (e.g. CAR-expressing T cells), and/or Compound C. In some embodiments, assessment is made prior to, during, during the course of, or after performing any of the methods provided herein. In some embodiments, the assessment is made prior to performing the methods provided herein. In some embodiments, assessment is made after performing one or more steps of the methods provided herein. In some embodiments, the assessment is performed prior to administration of administration of one or more steps of the provided combination therapy, for example, to screen and identify patients suitable and/or susceptible to receive the combination therapy. In some embodiments, the assessment is performed during, during the course of, or subsequent to administration of one or more steps of the provided combination therapy, for example, to assess the intermediate or final treatment outcome, e.g., to determine the efficacy of the treatment and/or to determine whether to continue or repeat the treatments and/or to determine whether to administer the remaining steps of the combination therapy.

In some embodiments, treatment of outcomes includes improved immune function, e.g., immune function of the T cells administered for cell based therapy and/or of the endogenous T cells in the body. In some embodiments, exemplary treatment outcomes include, but are not limited to, enhanced T cell proliferation, enhanced T cell functional activity, changes in immune cell phenotypic marker expression, such as such features being associated with the engineered T cells, e.g. CAR-T cells, administered to the subject. In some embodiments, exemplary treatment outcomes include decreased disease burden, e.g., tumor burden, improved clinical outcomes and/or enhanced efficacy of therapy.

In some embodiments, the screening step and/or assessment of treatment of outcomes includes assessing the survival and/or function of the T cells administered for cell based therapy. In some embodiments, the screening step and/or assessment of treatment of outcomes includes assessing the levels of cytokines or growth factors. In some embodiments, the screening step and/or assessment of treatment of outcomes includes assessing disease burden and/or improvements, e.g., assessing tumor burden and/or clinical outcomes. In some embodiments, either of the screening step and/or assessment of treatment of outcomes can include any of the assessment methods and/or assays described herein and/or known, and can be performed one or more times, e.g., prior to, during, during the course of, or subsequently to administration of one or more steps of the combination therapy. Exemplary sets of parameters associated with a treatment outcome, which can be assessed in some embodiments of the methods provided herein, include peripheral blood immune cell population profile and/or tumor burden.

In some embodiments, the methods affect efficacy of the cell therapy in the subject. In some embodiments, the persistence, expansion, and/or presence of recombinant receptor-expressing, e.g., CAR-expressing, cells in the subject following administration of the dose of cells in the method with Compound C is greater as compared to that achieved via a method without the administration of Compound C. In some embodiments, expansion and/or persistence in the subject of the administered T cell therapy, e.g., CAR-expressing T cells is assessed as compared to a method in which the T cell therapy is administered to the subject in the absence of Compound C. In some embodiments, the methods result in the administered T cells exhibiting increased or prolonged expansion and/or persistence in the subject as compared to a method in which the T cell therapy is administered to the subject in the absence of Compound C.

In some embodiments, the administration of Compound C decreases disease burden, e.g., tumor burden, in the subject as compared to a method in which the dose of cells expressing the recombinant receptor is administered to the subject in the absence of Compound C. In some embodiments, the administration of Compound C decreases blast marrow in the subject as compared to a method in which the dose of cells expressing the recombinant receptor is administered to the subject in the absence of Compound C. In some embodiments, the administration of Compound C results in improved clinical outcomes, e.g., objective response rate (ORR), progression-free survival (PFS) and overall survival (OS), compared to a method in which the dose of cells expressing the recombinant receptor is administered to the subject in the absence of Compound C.

In some embodiments, the subject can be screened prior to the administration of one or more steps of the combination therapy. For example, the subject can be screened for characteristics of the disease and/or disease burden, e.g., tumor burden, prior to administration of the combination therapy, to determine suitability, responsiveness and/or susceptibility to administering the combination therapy. In some embodiments, the screening step and/or assessment of treatment outcomes can be used to determine the dose, frequency, duration, timing and/or order of the combination therapy provided herein.

In some embodiments, the subject can be screened after administration of one of the steps of the combination therapy, to determine and identify subjects to receive the remaining steps of the combination therapy and/or to monitor efficacy of the therapy. In some embodiments, the number, level or amount of administered T cells and/or proliferation and/or activity of the administered T cells is assessed prior to administration and/or after administration of Compound C.

In some embodiments, a change and/or an alteration, e.g., an increase, an elevation, a decrease or a reduction, in levels, values or measurements of a parameter or outcome compared to the levels, values or measurements of the same parameter or outcome in a different time point of assessment, a different condition, a reference point and/or a different subject is determined or assessed. For example, in some embodiments, a fold change, e.g., an increase or decrease, in particular parameters, e.g., number of engineered T cells in a sample, compared to the same parameter in a different condition, e.g., before administration of Compound C can be determined. In some embodiments, the levels, values or measurements of two or more parameters are determined, and relative levels are compared. In some embodiments, the determined levels, values or measurements of parameters are compared to the levels, values or measurements from a control sample or an untreated sample. In some embodiments, the determined levels, values or measurements of parameters are compared to the levels from a sample from the same subject but at a different time point. The values obtained in the quantification of individual parameter can be combined for the purpose of disease assessment, e.g., by forming an arithmetical or logical operation on the levels, values or measurements of parameters by using multi-parametric analysis. In some embodiments, a ratio of two or more specific parameters can be calculated.

A. T cell Exposure, Persistence and Proliferation

In some embodiments, the parameter associated with therapy or a treatment outcome, which include parameters that can be assessed for the screening steps and/or assessment of treatment of outcomes and/or monitoring treatment outcomes, is or includes assessment of the exposure, persistence and proliferation of the T cells, e.g., T cells administered for the T cell based therapy. In some embodiments, the increased exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells used for the immunotherapy, e.g. T cell therapy, before or after administering one or more steps of the combination therapy provided herein.

In some embodiments, the administration of Compound C is designed to promote exposure of the subject to the cells, e.g., T cells administered for T cell based therapy, such as by promoting their expansion and/or persistence over time. In some embodiments, the T cell therapy exhibits increased or prolonged expansion and/or persistence in the subject as compared to a method in which the T cell therapy is administered to the subject in the absence of Compound C.

In some embodiments, the provided methods increase exposure of the subject to the administered cells (e.g., increased number of cells or duration over time) and/or improve efficacy and therapeutic outcomes of the immunotherapy, e.g. T cell therapy. In some aspects, the methods are advantageous in that a greater and/or longer degree of exposure to the cells expressing the recombinant receptors, e.g., CAR-expressing cells, improves treatment outcomes as compared with other methods. Such outcomes may include patient survival and remission, even in individuals with severe tumor burden.

In some embodiments, the administration of Compound C can increase the maximum, total, and/or duration of exposure to the cells, e.g. T cells administered for the T cell based therapy, in the subject as compared to administration of the T cells alone in the absence of Compound C. In some aspects, administration of Compound C, in the context of high disease burden (and thus higher amounts of antigen) and/or a more aggressive or resistant B cell malignancy enhances efficacy as compared with administration of the T cells alone in the absence of Compound C in the same context, which may result in immunosuppression, anergy and/or exhaustion which may prevent expansion and/or persistence of the cells.

In some embodiments, the presence and/or amount of cells expressing the recombinant receptor (e.g., CAR-expressing cells administered for T cell based therapy) in the subject following the administration of the T cells and before, during and/or after the administration of Compound C is detected. In some aspects, quantitative PCR (qPCR) is used to assess the quantity of cells expressing the recombinant receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid encoding the receptor, e.g., CAR, per microgram of DNA, e.g., total DNA obtained from a sample, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample.

In some embodiments, the cells are detected in the subject at or at least at 4, 7, 10, 14, 18, 21, 24, 27, or 28 days following the administration of the T cells, e.g., CAR-expressing T cells. In some aspects, the cells are detected at or at least at 2, 4, or 6 weeks following, or 3, 6, or 12, 18, or 24, or 30 or 36 months, or 1, 2, 3, 4, 5, or more years, following the administration of the T cells.

In some embodiments, the persistence of receptor-expressing cells (e.g. CAR-expressing cells) in the subject by the methods, following the administration of the T cells, e.g., CAR-expressing T cells and/or Compound C, is greater as compared to that which would be achieved by alternative methods such as those involving the administration of the immunotherapy alone, e.g., administration the T cells, e.g., CAR-expressing T cells, in the absence of Compound C.

The exposure, e.g., number of cells, e.g. T cells administered for T cell therapy, indicative of expansion and/or persistence, may be stated in terms of maximum numbers of the cells to which the subject is exposed, duration of detectable cells or cells above a certain number or percentage, area under the curve for number of cells over time, and/or combinations thereof and indicators thereof. Such outcomes may be assessed using known methods, such as qPCR to detect copy number of nucleic acid encoding the recombinant receptor compared to total amount of nucleic acid or DNA in the particular sample, e.g., blood, serum, plasma or tissue, such as a tumor sample, and/or flow cytometric assays detecting cells expressing the receptor generally using antibodies specific for the receptors. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor.

In some aspects, increased exposure of the subject to the cells includes increased expansion of the cells. In some embodiments, the receptor expressing cells, e.g. CAR-expressing cells, expand in the subject following administration of the T cells, e.g., CAR-expressing T cells, and/or following administration of Compound C. In some aspects, the methods result in greater expansion of the cells compared with other methods, such as those involving the administration of the T cells, e.g., CAR-expressing T cells, in the absence of administering Compound C.

In some aspects, the method results in high in vivo proliferation of the administered cells, for example, as measured by flow cytometry. In some aspects, high peak proportions of the cells are detected. For example, in some embodiments, at a peak or maximum level following the administration of the T cells, e.g., CAR-expressing T cells and/or Compound C, in the blood or disease-site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some embodiments, the method results in a maximum concentration, in the blood or serum or other bodily fluid or organ or tissue of the subject, of at least 100, 500, 1000, 1500, 2000, 5000, 10,000 or 15,000 copies of or nucleic acid encoding the receptor, e.g., the CAR, per microgram of DNA, or at least 0.1, 0.2, 0.3, 0.4, 0.5, 0.6, 0.7, 0.8, or 0.9 receptor-expressing, e.g., CAR-expressing cells per total number of peripheral blood mononuclear cells (PBMCs), total number of mononuclear cells, total number of T cells, or total number of microliters. In some embodiments, the cells expressing the receptor are detected as at least 10, 20, 30, 40, 50, or 60% of total PBMCs in the blood of the subject, and/or at such a level for at least 1, 2, 3, 4, 5, 6, 7, 8, 9, 10, 11, 12, 24, 36, 48, or 52 weeks following the T cells, e.g., CAR-expressing T cells and/or the Compound C, or for 1, 2, 3, 4, or 5, or more years following such administration.

In some aspects, the method results in at least a 2-fold, at least a 4-fold, at least a 10-fold, or at least a 20-fold increase in copies of nucleic acid encoding the recombinant receptor, e.g., CAR, per microgram of DNA, e.g., in the serum, plasma, blood or tissue, e.g., tumor sample, of the subject.

In some embodiments, cells expressing the receptor are detectable in the serum, plasma, blood or tissue, e.g., tumor sample, of the subject, e.g., by a specified method, such as qPCR or flow cytometry-based detection method, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more days following administration of the T cells, e.g., CAR-expressing T cells, or after administration of Compound C, for at least at or about 2, 3, 4, 5, 6, 7, 8, 9, 10, 11, 12, 13, 14, 15, 16, 17, 18, 19, 20, 21, 22, 23, or 24 or more weeks following the administration of the T cells, e.g., CAR-expressing T cells, and/or Compound C.

In some aspects, at least about 1×10², at least about 1×10³, at least about 1×10⁴, at least about 1×10⁵, or at least about 1×10⁶ or at least about 5×10⁶ or at least about 1×10⁷ or at least about 5×10⁷ or at least about 1×10⁸ recombinant receptor-expressing, e.g., CAR-expressing cells, and/or at least 10, 25, 50, 100, 200, 300, 400, or 500, or 1000 receptor-expressing cells per microliter, e.g., at least 10 per microliter, are detectable or are present in the subject or fluid, plasma, serum, tissue, or compartment thereof, such as in the blood, e.g., peripheral blood, or disease site, e.g., tumor, thereof. In some embodiments, such a number or concentration of cells is detectable in the subject for at least about 20 days, at least about 40 days, or at least about 60 days, or at least about 3, 4, 5, 6, 7, 8, 9, 10, 11, or 12 months, or at least 2 or 3 years, following administration of the T cells, e.g., CAR-expressing T cells, and/or following the administration of Compound C. Such cell numbers may be as detected by flow cytometry-based or quantitative PCR-based methods and extrapolation to total cell numbers using known methods. See, e.g., Brentjens et al., Sci Transl Med. 2013 5(177), Park et al, Molecular Therapy 15(4):825-833 (2007), Savoldo et al., JCI 121(5):1822-1826 (2011), Davila et al., (2013) PLoS ONE 8(4):e61338, Davila et al., Oncoimmunology 1(9): 1577-1583 (2012), Lamers, Blood 2011 117:72-82, Jensen et al., Biol Blood Marrow Transplant 2010 September; 16(9): 1245-1256, Brentjens et al., Blood 2011 118(18):4817-4828.

In some aspects, the copy number of nucleic acid encoding the recombinant receptor, e.g., vector copy number, per 100 cells, for example in the peripheral blood or bone marrow or other compartment, as measured by immunohistochemistry, PCR, and/or flow cytometry, is at least 0.01, at least 0.1, at least 1, or at least 10, at about 1 week, about 2 weeks, about 3 weeks, about 4 weeks, about 5 weeks, or at least about 6 weeks, or at least about 2, 3, 4, 5, 6, 7, 8. 9, 10, 11, or 12 months or at least 2 or 3 years following administration of the cells, e.g., CAR-expressing T cells, and/or Compound C. In some embodiments, the copy number of the vector expressing the receptor, e.g. CAR, per microgram of genomic DNA is at least 100, at least 1000, at least 5000, or at least 10,000, or at least 15,000 or at least 20,000 at a time about 1 week, about 2 weeks, about 3 weeks, or at least about 4 weeks following administration of the T cells, e.g., CAR-expressing T cells, or Compound C, or at least 2, 3, 4, 5, 6, 7, 8, 9, 10, 11, or 12 months or at least 2 or 3 years following such administration.

In some aspects, the receptor, e.g. CAR, expressed by the cells, is detectable by quantitative PCR (qPCR) or by flow cytometry in the subject, plasma, serum, blood, tissue and/or disease site thereof, e.g., tumor site, at a time that is at least about 3 months, at least about 6 months, at least about 12 months, at least about 1 year, at least about 2 years, at least about 3 years, or more than 3 years, following the administration of the cells, e.g., following the initiation of the administration of the T cells, e.g., CAR-expressing T cells, and/or Compound C.

In some embodiments, the area under the curve (AUC) for concentration of receptor- (e.g., CAR-) expressing cells in a fluid, plasma, serum, blood, tissue, organ and/or disease site, e.g. tumor site, of the subject over time following the administration of the T cells, e.g., CAR-expressing T cells and/or Compound C, is greater as compared to that achieved via an alternative dosing regimen where the subject is administered the T cells, e.g., CAR-expressing T cells, in the absence of administering Compound C.

In some aspects, the method results in high in vivo proliferation of the administered cells, for example, as measured by flow cytometry. In some aspects, high peak proportions of the cells are detected. For example, in some embodiments, at a peak or maximum level following the T cells, e.g., CAR-expressing T cells and/or Compound C, in the blood, plasma, serum, tissue or disease site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some aspects, the increased or prolonged expansion and/or persistence of the dose of cells in the subject administered with Compound C is associated with a benefit in tumor related outcomes in the subject. In some embodiments, the tumor related outcome includes a decrease in tumor burden or a decrease in blast marrow in the subject. In some embodiments, the tumor burden is decreased by or by at least at or about 10, 20, 30, 40, 50, 60, 70, 80, 90, or 100 percent after administration of the method. In some embodiments, disease burden, tumor size, tumor volume, tumor mass, and/or tumor load or bulk is reduced following the dose of cells by at least at or about 50%, 60%, 70%, 80%, 90% or more compared a subject that has been treated with a method that does not involve the administration of Compound C.

### B. T Cell Functional Activity

In some embodiments, parameters associated with therapy or a treatment outcome, which include parameters that can be assessed for the screening steps and/or assessment of treatment of outcomes and/or monitoring treatment outcomes, includes one or more of activity, phenotype, proliferation or function of T cells. In some embodiments, any of the known assays in the art for assessing the activity, phenotypes, proliferation and/or function of the T cells, e.g., T cells administered for T cell therapy, can be used. Prior to and/or subsequent to administration of the cells and/or Compound C, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known such as cytotoxicity assays described in, for example, Kochenderfer et al., J. Immunotherapy, 32(7): 689-702 (2009), and Herman et al., J. Immunological Methods, 285(1): 25-40 (2004).

In some embodiments, T cells, such as recombinant-expressing (e.g. CAR) T cells, can be assessed prior to and/or subsequent to administration of the cells and/or Compound C to assess or determine if the T cells exhibit features of exhaustion. In some cases, exhaustion can be assessed by monitoring loss of T cell function, such as reduced or decreased antigen-specific or antigen receptor-driven activity, such as a reduced or decreased ability to produce cytokines or to drive cytolytic activity against target antigen. In some cases, exhaustion also can be assessed by monitoring expression of surface markers on T cells (e.g. CD4 and/or CD4 T cells) that are associated with an exhaustion phenotype. Among exhaustion markers are inhibitory receptors such as PD-1, CTLA-4, LAG-3 and TIM-3.

In some embodiments, such a reduced or decreased activity is observed over time following administration to the subject and/or following long-term exposure to antigen.

In particular embodiments, the provided methods (i) to effect said increase in antigen-specific or antigen receptor-driven activity and (ii) to prevent, inhibit or delay said onset of exhaustion phenotype and/or to reverse said exhaustion phenotype. In some embodiments, the amount, duration and/or frequency is effective (i) to effect said increase in antigen-specific or antigen receptor-driven activity and (ii) to prevent, inhibit or delay said onset of exhaustion phenotype. In other embodiments, the amount, duration and/or frequency is effective (i) to effect said increase in antigen-specific or antigen receptor-driven activity and (ii) to prevent, inhibit or delay said onset of exhaustion phenotype and to reverse said exhaustion phenotype.

In some embodiments, the exhaustion phenotype, with reference to a T cell or population of T cells, comprises: an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or a decrease in the level or degree of an activity exhibited by said T cells or population of T cells upon exposure to an antigen or antigen receptor-specific agent, compared to a reference T cell population, under the same conditions. an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or a decrease in the level or degree of an activity exhibited by said T cells or population of T cells upon exposure to an antigen or antigen receptor-specific agent, compared to a reference T cell population, under the same conditions.

In certain embodiments, the biological activity of the cells is measured by assaying expression and/or secretion of one or more cytokines, such as CD107a, IFNγ, IL-2, GM-CSF and TNFα, and/or by assessing cytolytic activity.

In some embodiments, assays for the activity, phenotypes, proliferation and/or function of the T cells, e.g., T cells administered for T cell therapy include, but are not limited to, ELISPOT, ELISA, cellular proliferation, cytotoxic lymphocyte (CTL) assay, binding to the T cell epitope, antigen or ligand, or intracellular cytokine staining, proliferation assays, lymphokine secretion assays, direct cytotoxicity assays, and limiting dilution assays. In some embodiments, proliferative responses of the T cells can be measured, e.g. by incorporation of 3H-thymidine, BrdU (5-Bromo-2′-Deoxyuridine) or 2′-deoxy-5-ethynyluridine (EdU) into their DNA or dye dilution assays, using dyes such as carboxyfluorescein diacetate succinimidyl ester (CFSE), CellTrace Violet, or membrane dye PKH26.

In some embodiments, assessing the activity, phenotypes, proliferation and/or function of the T cells, e.g., T cells administered for T cell therapy, include measuring cytokine production from T cells, and/or measuring cytokine production in a biological sample from the subject, e.g., plasma, serum, blood, and/or tissue samples, e.g., tumor samples. In some cases, such measured cytokines can include, without limitation, interleukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). Assays to measure cytokines are well known, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample.

In some embodiments, assessing the activity, phenotypes, proliferation and/or function of the T cells, e.g., T cells administered for T cell therapy, include assessing cell phenotypes, e.g., expression of particular cell surface markers. In some embodiments, the T cells, e.g., T cells administered for T cell therapy, are assessed for expression of T cell activation markers, T cell exhaustion markers, and/or T cell differentiation markers. In some embodiments, the cell phenotype is assessed before administration. In some embodiments, the cell phenotype is assessed during, or after administration of cell therapy and/or Compound C. T cell activation markers, T cell exhaustion markers, and/or T cell differentiation markers for assessment include any markers known for particular subsets of T cells, e.g., CD25, CD38, human leukocyte antigen-DR (HLA-DR), CD69, CD44, CD137, KLRG1, CD62L^{low}, CCR7^{low}, CD71, CD2, CD54, CD58, CD244, CD160, programmed cell death protein 1 (PD-1), lymphocyte activation gene 3 protein (LAG-3), T-cell immunoglobulin domain and mucin domain protein 3 (TIM-3), cytotoxic T lymphocyte antigen-4 (CTLA-4), band T lymphocyte attenuator (BTLA) and/or T-cell immunoglobulin and immunoreceptor tyrosine-based inhibitory motif domain (TIGIT) (see, e.g., Liu et al., Cell Death and Disease (2015) 6, e1792). In some embodiments, the exhaustion marker is any one or more of PD-1, CTLA-4, TIM-3, LAG-3, BTLA, 2B4, CD160, CD39, VISTA, and TIGIT In some embodiments, the assessed cell surface marker is CD25, PD-1 and/or TIM-3. In some embodiments, the assessed cell surface marker is CD25.

In some aspects, detecting the expression levels includes performing an in vitro assay. In some embodiments, the in vitro assay is an immunoassay, an aptamer-based assay, a histological or cytological assay, or an mRNA expression level assay. In some embodiments, the parameter or parameters for one or more of each of the one or more factors, effectors, enzymes and/or surface markers are detected by an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay. In some embodiments, detection of cytokines and/or surface markers is determined using a binding reagent that specifically binds to at least one biomarker. In some cases, the binding reagent is an antibody or antigen-binding fragment thereof, an aptamer or a nucleic acid probe.

In some embodiments, the administration of Compound C increases the level of circulating CAR T cells.

### C. Response, Efficacy and Survival

In some embodiments, parameters associated with therapy or a treatment outcome, which include parameters that can be assessed for the screening steps and/or assessment of treatment of outcomes and/or monitoring treatment outcomes, includes tumor or disease burden. The administration of the immunotherapy, such as a T cell therapy (e.g. CAR-expressing T cells) and/or Compound C, can reduce or prevent the expansion or burden of the disease or condition in the subject. For example, where the disease or condition is a tumor, the methods generally reduce tumor size, bulk, metastasis, percentage of blasts in the bone marrow or molecularly detectable B cell malignancy and/or improve prognosis or survival or other symptom associated with tumor burden.

In some aspects, the administration in accord with the provided methods, and/or with the provided articles of manufacture or compositions, generally reduces or prevents the expansion or burden of the disease or condition in the subject. For example, where the disease or condition is a tumor, the methods generally reduce tumor size, bulk, metastasis, percentage of blasts in the bone marrow or molecularly detectable B cell malignancy and/or improve prognosis or survival or other symptom associated with tumor burden.

In some embodiments, the provided methods result in a decreased tumor burden in treated subjects compared to alternative methods in which the immunotherapy, such as a T cell therapy (e.g. CAR-expressing T cells) is given without administration of Compound C. It is not necessary that the tumor burden actually be reduced in all subjects receiving the combination therapy, but that tumor burden is reduced on average in subjects treated, such as based on clinical data, in which a majority of subjects treated with such a combination therapy exhibit a reduced tumor burden, such as at least 50%, 60%, 70%, 80%, 90%, 95% or more of subjects treated with the combination therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In some embodiments, the subject has a lymphoma or a leukemia. The extent of disease burden can be determined by assessment of residual leukemia in blood or bone marrow. In some embodiments, the subject has a non-Hodgkin lymphoma (NHL), an acute lymphoblastic leukemia (ALL), a chronic lymphocytic leukemia (CLL), or a diffuse large B-cell lymphoma (DLBCL). In some embodiments, the subject has a MM or a DBCBL.

In some aspects, response rates in subjects, such as subjects with NHL, are based on the Lugano criteria. (Cheson et al., (2014) JCO., 32(27):3059-3067; Johnson et al., (2015) Radiology 2:323-338; Cheson, B. D. (2015) Chin. Clin. Oncol. 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake ≤mediastinum; 3, uptake >mediastinum but ≤liver; 4, uptake moderately >liver; 5, uptake markedly higher than liver and/or new lesions; X, new areas of uptake unlikely to be related to lymphoma.

In some aspects, a complete response as described using the Lugano criteria involves a complete metabolic response and a complete radiologic response at various measureable sites. In some aspects, these sites include lymph nodes and extralymphatic sites, wherein a CR is described as a score of 1, 2, or 3 with or without a residual mass on the 5-point scale, when PET-CT is used. In some aspects, in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. In some aspects, response is assessed in the lymph nodes using CT, wherein a CR is described as no extralymphatic sites of disease and target nodes/nodal masses must regress to ≤1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology, which if indeterminate should be IHC negative. Further sites may include assessment of organ enlargement, which should regress to normal. In some aspects, nonmeasured lesions and new lesions are assessed, which in the case of CR should be absent (Cheson et al., (2014) JCO., 32(27): 3059-3067; Johnson et al., (2015) Radiology 2:323-338; Cheson, B. D. (2015) Chin. Clin. Oncol. 4(1):5).

In some aspects, a partial response (PR) as described using the Lugano criteria involves a partial metabolic and/or radiological response at various measureable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease. In some aspects, response is assessed in the lymph nodes using CT, wherein a PR is described as ≥50% decrease in SPD of up to 6 target measureable nodes and extranodal sites. If a lesion is too small to measure on CT, 5 mm×5 mm is assigned as the default value; if the lesion is no longer visible, the value is 0 mm×0 mm; for a node >5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, nonmeasured lesions and new lesions are assessed, which in the case of PR should be absent/normal, regressed, but no increase. No response/stable disease (SD) or progressive disease (PD) can also be measured using PET-CT and/or CT based assessments. (Cheson et al., (2014) JCO., 32(27): 3059-3067; Johnson et al., (2015) Radiology 2:323-338; Cheson, B. D. (2015) Chin. Clin. Oncol., 4(1):5).

In some respects, progression-free survival (PFS) is described as the length of time during and after the treatment of a disease, such as a B cell malignancy, that a subject lives with the disease but it does not get worse. In some aspects, objective response (OR) is described as a measurable response. In some aspects, objective response rate (ORR) is described as the proportion of patients who achieved CR or PR. In some aspects, overall survival (OS) is described as the length of time from either the date of diagnosis or the start of treatment for a disease, such as a B cell malignancy, that subjects diagnosed with the disease are still alive. In some aspects, event-free survival (EFS) is described as the length of time after treatment for a B cell malignancy ends that the subject remains free of certain complications or events that the treatment was intended to prevent or delay. These events may include the return of the B cell malignancy or the onset of certain symptoms, such as bone pain from B cell malignancy that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months or greater than 6 months.

In some aspects, the RECIST criteria is used to determine objective tumor response. (Eisenhauer et al., European Journal of Cancer 45 (2009) 228-247.) In some aspects, the RECIST criteria is used to determine objective tumor response for target lesions. In some respects, a complete response as determined using RECIST criteria is described as the disappearance of all target lesions and any pathological lymph nodes (whether target or non-target) must have reduction in short axis to <10 mm. In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

In the case of MM, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/1); renal insufficiency attributable to myeloma; anemia (hemoglobin <10 g/dl); and/or bone lesions (lytic lesions or osteoporosis with compression fractures)).

In the case of DLBCL, exemplary parameters to assess the extent of disease burden include such parameters as cellular morphology (e.g., centroblastic, immunoblastic, and anaplastic cells), gene expression, miRNA expression and protein expression (e.g., expression of BCL2, BCL6, MUM1, LMO2, MYC, and p21).

In some aspects, response rates in subjects, such as subjects with CLL, are based on the International Workshop on Chronic Lymphocytic Leukemia (IWCLL) response criteria (Hallek, et al., Blood 2008, Jun. 15; 111(12): 5446-5456). In some aspects, these criteria are described as follows: complete remission (CR), which in some aspects requires the absence of peripheral blood clonal lymphocytes by immunophenotyping, absence of lymphadenopathy, absence of hepatomegaly or splenomegaly, absence of constitutional symptoms and satisfactory blood counts; complete remission with incomplete marrow recovery (CRi), which in some aspects is described as CR above, but without normal blood counts; partial remission (PR), which in some aspects is described as ≥50% fall in lymphocyte count, ≥50% reduction in lymphadenopathy or ≥50% reduction in liver or spleen, together with improvement in peripheral blood counts; progressive disease (PD), which in some aspects is described as ≥50% rise in lymphocyte count to >5×10⁹/L, ≥50% increase in lymphadenopathy, ≥50% increase in liver or spleen size, Richter's transformation, or new cytopenias due to CLL; and stable disease, which in some aspects is described as not meeting criteria for CR, CRi, PR or PD.

In some embodiments, the subjects exhibits a CR or OR if, within 1 month of the administration of the dose of cells, lymph nodes in the subject are less than at or about 20 mm in size, less than at or about 10 mm in size or less than at or about 10 mm in size.

In some embodiments, an index clone of the CLL is not detected in the bone marrow of the subject (or in the bone marrow of greater than 50%, 60%, 70%, 80%, 90% or more of the subjects treated according to the methods. In some embodiments, an index clone of the CLL is assessed by IgH deep sequencing. In some embodiments, the index clone is not detected at a time that is at or about or at least at or about 1, 2, 3, 4, 5, 6, 12, 18 or 24 months following the administration of the cells.

In some embodiments, a subject exhibits morphologic disease if there are greater than or equal to 5% blasts in the bone marrow, for example, as detected by light microscopy, such as greater than or equal to 10% blasts in the bone marrow, greater than or equal to 20% blasts in the bone marrow, greater than or equal to 30% blasts in the bone marrow, greater than or equal to 40% blasts in the bone marrow or greater than or equal to 50% blasts in the bone marrow. In some embodiments, a subject exhibits complete or clinical remission if there are less than 5% blasts in the bone marrow.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable B cell malignancy. In some embodiments, molecularly detectable B cell malignancy can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify B cell malignancy cell based on leukemia-specific immunophenotypes. In some embodiments, molecular detection of B cell malignancy can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In the case of leukemia, the extent of disease burden can be determined by assessment of residual leukemia in blood or bone marrow. In some embodiments, a subject exhibits morphologic disease if there are greater than or equal to 5% blasts in the bone marrow, for example, as detected by light microscopy. In some embodiments, a subject exhibits complete or clinical remission if there are less than 5% blasts in the bone marrow.

In some embodiments, for leukemia, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable B cell malignancy. In some embodiments, molecularly detectable B cell malignancy can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify B cell malignancy cell based on leukemia-specific immunophenotypes. In some embodiments, molecular detection of B cell malignancy can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In some embodiments, the methods and/or administration of a cell therapy, such as a T cell therapy (e.g. CAR-expressing T cells) and/or Compound C decrease(s) disease burden as compared with disease burden at a time immediately prior to the administration of the immunotherapy, e.g., T cell therapy and/or Compound C.

In some aspects, administration of the immunotherapy, e.g. T cell therapy and/or Compound C, may prevent an increase in disease burden, and this may be evidenced by no change in disease burden.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives immunotherapy, e.g. T cell therapy alone, in the absence of administration of Compound C. In some embodiments, disease burden is reduced to a greater extent or for a greater duration following the combination therapy of administration of the immunotherapy, e.g., T cell therapy, and Compound C, compared to the reduction that would be effected by administering each of the agent alone, e.g., administering Compound C to a subject having not received the immunotherapy, e.g. T cell therapy; or administering the immunotherapy, e.g. T cell therapy, to a subject having not received Compound C.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). Specific thresholds for the parameters can be set to determine the efficacy of the method of combination therapy provided herein.

In some aspects, disease burden is measured or detected prior to administration of the immunotherapy, e.g. T cell therapy, following the administration of the immunotherapy, e.g. T cell therapy but prior to administration of Compound C, and/or following the administration of both the immunotherapy, e.g. T cell therapy and Compound C. In the context of multiple administration of one or more steps of the combination therapy, disease burden in some embodiments may be measured prior to, or following administration of any of the steps, doses and/or cycles of administration, or at a time between administration of any of the steps, doses and/or cycles of administration.

In some embodiments, the burden is decreased by or by at least at or about 10, 20, 30, 40, 50, 60, 70, 80, 90, or 100 percent by the provided methods compared to immediately prior to the administration of Compound C and the immunotherapy, e.g. T cell therapy. In some embodiments, disease burden, tumor size, tumor volume, tumor mass, and/or tumor load or bulk is reduced following administration of the immunotherapy, e.g. T cell therapy and Compound C, by at least at or about 10, 20, 30, 40, 50, 60, 70, 80, 90% or more compared to that immediately prior to the administration of the immunotherapy, e.g. T cell therapy and/or Compound C.

In some embodiments, reduction of disease burden by the method comprises an induction in morphologic complete remission, for example, as assessed at 1 month, 2 months, 3 months, 4 months, 5 months, 6 months, or more than 6 months, after administration of, e.g., initiation of, the combination therapy.

In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of combination therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the method of combination therapy, is less than about 80%, less than about 70%, less than about 60%, less than about 50%, less than about 40%, less than about 30%, less than about 20%, or less than about 10%.

In some cases, the pharmacokinetics of administered cells, e.g., adoptively transferred cells are determined to assess the availability, e.g., bioavailability of the administered cells. Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (e.g., a CAR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the number of CAR+ T cells in a biological sample obtained from the patient, e.g., blood, can be determined at a period of time after administration of the cell therapy, e.g., to determine the pharmacokinetics of the cells. In some embodiments, number of CAR+ T cells, optionally CAR+CD8+ T cells and/or CAR+CD4+ T cells, detectable in the blood of the subject, or in a majority of subjects so treated by the method, is greater than 1 cells per μL, greater than 5 cells per μL or greater than per 10 cells per μL.

### IV. TOXICITY AND ADVERSE OUTCOMES

In embodiments of the provided methods, the subject is monitored for toxicity or other adverse outcome, including treatment related outcomes, e.g., development of neutropenia, cytokine release syndrome (CRS) or neurotoxicity (NT), in subjects administered the provided combination therapy comprising a cell therapy (e.g., a T cell therapy) and Compound C. In some embodiments, the provided methods are carried out to reduce the risk of a toxic outcome or symptom, toxicity-promoting profile, factor, or property, such as a symptom or outcome associated with or indicative of severe neutropenia, severe cytokine release syndrome (CRS) or severe neurotoxicity.

In some embodiments, the provided methods do not result in a high rate or likelihood of toxicity or toxic outcomes, or reduces the rate or likelihood of toxicity or toxic outcomes, such as severe neurotoxicity (NT) or severe cytokine release syndrome (CRS), such as compared to certain other cell therapies. In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxicity. In some embodiments, no more than 50% of subjects treated (e.g. at least 60%, at least 70%, at least 80%, at least 90% or more of the subjects treated) a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells.

In some embodiments, the provided methods do not result in a high rate or likelihood of toxicity or toxic outcomes, or reduces the rate or likelihood of toxicity or toxic outcomes, such as severe neurotoxicity (NT) or severe cytokine release syndrome (CRS), such as compared to certain other cell therapies. In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxicity. In some embodiments, no more than 50% of subjects treated (e.g. at least 60%, at least 70%, at least 80%, at least 90% or more of the subjects treated) a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells.

### A. Cytokine Release Syndrome (CRS) and Neurotoxicity

In some aspects, the toxic outcome is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure. CRS may be treated using anti-inflammatory therapy such as an anti-IL-6 therapy, e.g., anti-IL-6 antibody, e.g., tocilizumab, or antibiotics or other agents as described.

Outcomes, signs and symptoms of CRS are known and include those described herein. In some embodiments, where a particular dosage regimen or administration effects or does not effect a given CRS-associated outcome, sign, or symptom, particular outcomes, signs, and symptoms and/or quantities or degrees thereof may be specified.

In the context of administering CAR-expressing cells, CRS typically occurs 6-20 days after infusion of cells that express a CAR. See Xu et al., Cancer Letters 343 (2014) 172-78. In some cases, CRS occurs less than 6 days or more than 20 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1B, IL-6, IL-8, and IL-10.

Exemplary outcomes associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly.

In some embodiments, outcomes associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures).

Exemplary CRS-related outcomes include increased or high serum levels of one or more factors, including cytokines and chemokines and other factors associated with CRS. Exemplary outcomes further include increases in synthesis or secretion of one or more of such factors. Such synthesis or secretion can be by the T cell or a cell that interacts with the T cell, such as an innate immune cell or B cell.

In some embodiments, the CRS-associated serum factors or CRS-related outcomes include inflammatory cytokines and/or chemokines, including interferon gamma (IFN-γ), TNF-a, IL-1β, IL-2, IL-6, IL-7, IL-8, IL-10, IL-12, sIL-2Ra, granulocyte macrophage colony stimulating factor (GM-CSF), macrophage inflammatory protein (MIP)-1, tumor necrosis factor alpha (TNFα), IL-6, and IL-10, IL-1B, IL-8, IL-2, MIP-1, Flt-3L, fracktalkine, and/or IL-5. In some embodiments, the factor or outcome includes C reactive protein (CRP). In addition to being an early and easily measurable risk factor for CRS, CRP also is a marker for cell expansion. In some embodiments, subjects that are measured to have high levels of CRP, such as ≥15 mg/dL, have CRS. In some embodiments, subjects that are measured to have high levels of CRP do not have CRS. In some embodiments, a measure of CRS includes a measure of CRP and another factor indicative of CRS.

In some embodiments, one or more inflammatory cytokines or chemokines are monitored before, during, or after CAR treatment and/or Compound C treatment. In some aspects, the one or more cytokines or chemokines include IFN-γ, TNF-α, IL-2, IL-1β, IL-6, IL-7, IL-8, IL-10, IL-12, sIL-2Rα, granulocyte macrophage colony stimulating factor (GM-CSF), or macrophage inflammatory protein (MIP). In some embodiments, IFN-γ, TNF-α, and IL-6 are monitored.

CRS criteria that appear to correlate with the onset of CRS to predict which patients are more likely to be at risk for developing sCRS have been developed (see Davilla et al. Science translational medicine. 2014; 6(224):224ra25). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. Other guidelines on the diagnosis and management of CRS are known (see e.g., Lee et al, Blood. 2014; 124(2): 188-95). In some embodiments, the criteria reflective of CRS grade are those detailed in Table 3 below.

TABLE 3
Exemplary Grading Criteria for CRS

| Grade | Description of Symptoms |
|---|---|
| 1 Mild | Not life-threatening, require only symptomatic treat- |
| | ment such as antipyretics and anti-emetics (e.g., |
| | fever, nausea, fatigue, headache, myalgias, malaise) |
| 2 Moderate | Require and respond to moderate intervention: |
| | □ Oxygen requirement < 40%, or |
| | □ Hypotension responsive to fluids or low dose of a |
| | single vasopressor, or |
| | □ Grade 2 organ toxicity (by CTCAE v4.0) |
| 3 Severe | Require and respond to aggressive intervention: |
| | □ Oxygen requirement ≥ 40%, or |
| | □ Hypotension requiring high dose of a single vasopres- |
| | sor (e.g., norepinephrine ≥ 20 μg/kg/min, dopamine ≥ |
| | 10 μg/kg/min, phenylephrine ≥ 200 μg/kg/min, or |
| | epinephrine ≥ 10 μg/kg/min), or |
| | □ Hypotension requiring multiple vasopressors (e.g., |
| | vasopressin + one of the above agents, or combination |
| | vasopressors equivalent to ≥20 μg/kg/min norepi- |
| | nephrine), or |
| | □ Grade 3 organ toxicity or Grade 4 transaminitis (by |
| | CTCAE v4.0) |
| 4 Life- | Life-threatening: |
| threatening | □ Requirement for ventilator support, or |
| | □ Grade 4 organ toxicity (excluding transaminitis) |
| 5 Fatal | Death |

In some embodiments, a subject is deemed to develop “severe CRS” (“sCRS”) in response to or secondary to administration of a cell therapy or dose of cells thereof, if, following administration, the subject displays: (1) fever of at least 38 degrees Celsius for at least three days; (2) cytokine elevation that includes either (a) a max fold change of at least 75 for at least two of the following group of seven cytokines compared to the level immediately following the administration: interferon gamma (IFNγ), GM-CSF, IL-6, IL-10, Flt-3L, fracktalkine, and IL-5 and/or (b) a max fold change of at least 250 for at least one of the following group of seven cytokines compared to the level immediately following the administration: interferon gamma (IFNγ), GM-CSF, IL-6, IL-10, Flt-3L, fracktalkine, and IL-5; and (c) at least one clinical sign of toxicity such as hypotension (requiring at least one intravenous vasoactive pressor) or hypoxia (PO₂<90%) or one or more neurologic disorder(s) (including mental status changes, obtundation, and/or seizures). In some embodiments, severe CRS includes CRS with a grade of 3 or greater, such as set forth in Table 4.

In some embodiments, outcomes associated with severe CRS or grade 3 CRS or greater, such as grade 4 or greater, include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, severe CRS includes CRS that requires management or care in the intensive care unit (ICU).

In some embodiments, the CRS, such as severe CRS, encompasses a combination of (1) persistent fever (fever of at least 38 degrees Celsius for at least three days) and (2) a serum level of CRP of at least at or about 20 mg/dL. In some embodiments, the CRS encompasses hypotension requiring the use of two or more vasopressors or respiratory failure requiring mechanical ventilation. In some embodiments, the dosage of vasopressors is increased in a second or subsequent administration.

In some embodiments, severe CRS or grade 3 CRS encompasses an increase in alanine aminotransferase, an increase in aspartate aminotransferase, chills, febrile neutropenia, headache, left ventricular dysfunction, encephalopathy, hydrocephalus, and/or tremor.

The method of measuring or detecting the various outcomes may be specified.

In some aspects, the toxic outcome of a therapy, such as a cell therapy, is or is associated with or indicative of neurotoxicity or severe neurotoxicity. In some embodiments, symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03).

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, a subject is deemed to develop “severe neurotoxicity” in response to or secondary to administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table 4. In some embodiments, a severe neurotoxicity is deemed to be a prolonged grade 3 if symptoms or grade 3 neurotoxicity last for 10 days or longer.

TABLE 4
Exemplary Grading Criteria for Neurotoxicity

| Grade | Description of Symptoms |
|---|---|
| 1 Asymptomatic | Mild or asymptomatic symptoms |
| or Mild | |
| 2 Moderate | Presence of symptoms that limit instrumental activi- |
| | ties of daily living (ADL), such as preparing meals, |
| | shopping for groceries or clothes, using the telephone, |
| | managing money |
| 3 Severe | Presence of symptoms that limit self-care ADL, such |
| | as bathing, dressing and undressing, feeding self, |
| | using the toilet, taking medications |
| 4 Life- | Symptoms that are life-threatening, requiring urgent |
| threatening | intervention |
| 5 Fatal | Death |

In some embodiments, the methods reduce symptoms associated with CRS or neurotoxicity compared to other methods. In some aspects, the provided methods reduce symptoms, outcomes or factors associated with CRS, including symptoms, outcomes or factors associated with severe CRS or grade 3 or higher CRS, compared to other methods. For example, subjects treated according to the present methods may lack detectable and/or have reduced symptoms, outcomes or factors of CRS, e.g. severe CRS or grade 3 or higher CRS, such as any described, e.g. set forth in Table 3. In some embodiments, subjects treated according to the present methods may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods. In some embodiments, subjects treated according to the present methods may have reduced symptoms associated with peripheral motor neuropathy, peripheral sensory neuropathy, dysesthesia, neuralgia or paresthesia.

In some embodiments, the methods reduce outcomes associated with neurotoxicity including damages to the nervous system and/or brain, such as the death of neurons. In some aspects, the methods reduce the level of factors associated with neurotoxicity such as beta amyloid (Aβ), glutamate, and oxygen radicals.

In some embodiments, the toxicity outcome is a dose-limiting toxicity (DLT). In some embodiments, the toxic outcome is the absence of a dose-limiting toxicity. In some embodiments, a dose-limiting toxicity (DLT) is defined as any grade 3 or higher toxicity as described or assessed by any known or published guidelines for assessing the particular toxicity, such as any described above and including the National Cancer Institute (NCI) Common Terminology Criteria for Adverse Events (CTCAE) version 4.0. In some embodiments, a dose-limiting toxicity (DLT) is defined when any of the events discussed below occurs following administration of the cell therapy (e.g., T cell therapy) and/or Compound C, the events including a) febrile neutropenia; b) Grade 4 neutropenia lasting about or more than about 7 days; c) Grade 3 or 4 thrombocytopenia with clinically significant bleeding; and d) Grade 4 thrombocytopenia lasting more than 24 hours.

In some embodiments, the provided embodiments result in a low rate or risk of developing a toxicity, e.g. CRS or neurotoxicity or severe CRS or neurotoxicity, e.g. grade 3 or higher CRS or neurotoxicity, such as observed with administering a dose of T cells in accord with the provided combination therapy, and/or with the provided articles of manufacture or compositions. In some cases, this permits administration of the cell therapy on an outpatient basis. In some embodiments, the administration of the cell therapy, e.g. dose of T cells (e.g. CAR+ T cells) in accord with the provided methods, and/or with the provided articles of manufacture or compositions, is performed on an outpatient basis or does not require admission to the subject to the hospital, such as admission to the hospital requiring an overnight stay.

In some aspects, subjects administered the cell therapy, e.g. dose of T cells (e.g. CAR+ T cells) in accord with the provided methods, and/or with the provided articles of manufacture or compositions, including subjects treated on an outpatient basis, are not administered an intervention for treating any toxicity prior to or with administration of the cell dose, unless or until the subject exhibits a sign or symptom of a toxicity, such as of a neurotoxicity or CRS.

In some embodiments, if a subject administered the cell therapy, e.g. dose of T cells (e.g. CAR+ T cells), including subjects treated on an outpatient basis, exhibits a fever the subject is given or is instructed to receive or administer a treatment to reduce the fever. In some embodiments, the fever in the subject is characterized as a body temperature of the subject that is (or is measured at) at or above a certain threshold temperature or level. In some aspects, the threshold temperature is that associated with at least a low-grade fever, with at least a moderate fever, and/or with at least a high-grade fever. In some embodiments, the threshold temperature is a particular temperature or range. For example, the threshold temperature may be at or about or at least at or about 38, 39, 40, 41, or 42 degrees Celsius, and/or may be a range of at or about 38 degrees Celsius to at or about 39 degrees Celsius, a range of at or about 39 degrees Celsius to at or about 40 degrees Celsius, a range of at or about 40 degrees Celsius to at or about 41 degrees, or a range of at or about 41 degrees Celsius to at or about 42 degrees Celsius.

In some embodiments, the treatment designed to reduce fever includes treatment with an antipyretic. An antipyretic may include any agent, composition, or ingredient, that reduces fever, such as one of any number of agents known to have antipyretic effects, such as NSAIDs (such as ibuprofen, naproxen, ketoprofen, and nimesulide), salicylates, such as aspirin, choline salicylate, magnesium salicylate, and sodium salicylate, paracetamol, acetaminophen, Metamizole, Nabumetone, Phenaxone, antipyrine, febrifuges. In some embodiments, the antipyretic is acetaminophen. In some embodiments, acetaminophen can be administered at a dose of 12.5 mg/kg orally or intravenously up to every four hours. In some embodiments, it is or comprises ibuprofen or aspirin.

In some embodiments, if the fever is a sustained fever, the subject is administered an alternative treatment for treating the toxicity. For subjects treated on an outpatient basis, the subject is instructed to return to the hospital if the subject has and/or is determined to or to have a sustained fever. In some embodiments, the subject has, and/or is determined to or considered to have, a sustained fever if he or she exhibits a fever at or above the relevant threshold temperature, and where the fever or body temperature of the subject is not reduced, or is not reduced by or by more than a specified amount (e.g., by more than 1° C., and generally does not fluctuate by about, or by more than about, 0.5° C., 0.4° C., 0.3° C., or 0.2° C.), following a specified treatment, such as a treatment designed to reduce fever such as treatment with an antipyreticm, e.g. NSAID or salicylates, e.g. ibuprofen, acetaminophen or aspirin. For example, a subject is considered to have a sustained fever if he or she exhibits or is determined to exhibit a fever of at least at or about 38 or 39 degrees Celsius, which is not reduced by or is not reduced by more than at or about 0.5° C., 0.4° C., 0.3° C., or 0.2° C., or by at or about 1%, 2%, 3%, 4%, or 5%, over a period of 6 hours, over a period of 8 hours, or over a period of 12 hours, or over a period of 24 hours, even following treatment with the antipyretic such as acetaminophen. In some embodiments, the dosage of the antipyretic is a dosage ordinarily effective in such as subject to reduce fever or fever of a particular type such as fever associated with a bacterial or viral infection, e.g., a localized or systemic infection.

In some embodiments, the subject has, and/or is determined to or considered to have, a sustained fever if he or she exhibits a fever at or above the relevant threshold temperature, and where the fever or body temperature of the subject does not fluctuate by about, or by more than about, 1° C., and generally does not fluctuate by about, or by more than about, 0.5° C., 0.4° C., 0.3° C., or 0.2° C. Such absence of fluctuation above or at a certain amount generally is measured over a given period of time (such as over a 24-hour, 12-hour, 8-hour, 6-hour, 3-hour, or 1-hour period of time, which may be measured from the first sign of fever or the first temperature above the indicated threshold). For example, in some embodiments, a subject is considered to or is determined to exhibit sustained fever if he or she exhibits a fever of at least at or about or at least at or about 38 or 39 degrees Celsius, which does not fluctuate in temperature by more than at or about 0.5° C., 0.4° C., 0.3° C., or 0.2° C., over a period of 6 hours, over a period of 8 hours, or over a period of 12 hours, or over a period of 24 hours.

In some embodiments, the fever is a sustained fever; in some aspects, the subject is treated at a time at which a subject has been determined to have a sustained fever, such as within one, two, three, four, five six, or fewer hours of such determination or of the first such determination following the initial therapy having the potential to induce the toxicity, such as the cell therapy, such as dose of T cells, e.g. CAR+ T cells.

In some embodiments, one or more interventions or agents for treating the toxicity, such as a toxicity-targeting therapies, is administered at a time at which or immediately after which the subject is determined to or confirmed to (such as is first determined or confirmed to) exhibit sustained fever, for example, as measured according to any of the aforementioned embodiments. In some embodiments, the one or more toxicity-targeting therapies is administered within a certain period of time of such confirmation or determination, such as within 30 minutes, 1 hour, 2 hours, 3 hours, 4 hours, 6 hours, or 8 hours thereof.

### B. Hematologic Toxicity

In some aspects, the toxic outcome is or is associated with or indicative of a hematologic toxicity, such as thrombocytopenia and/or neutropenia. In some cases, hematological toxicities, including thrombocytopenia and neutropenia, are graded according to Common Terminology Criteria for Adverse Events (Version 4.03; US National Cancer Institute, Bethesda, MD, USA). In some cases, a hematological toxicity, such as thrombocytopenia and/or neutropenia, is monitored before, during, and after the administration(s) of the Compound C. In some cases, a hematological toxicity, such as thrombocytopenia and/or neutropenia, is monitored prior to each administration of Compound C. In some cases, a hematological toxicity, such as thrombocytopenia and/or neutropenia, is monitored at least every 1, 2, 3, 4, 5, 6, or 7 days after administration of Compound C.

In some embodiments, a complete blood count is carried out to monitor levels of leukocytes (white blood cells) in the subject, including neutrophils and platelets. A variety of methods can be used carry out a complete blood cell (CBC) count and/or a leukocyte differential count. In some embodiments, a hematology analyzer is used.

Neutropenia is characterized by a reduction in the blood neutrophil count, often leading to increased susceptibility to bacterial and fungal infections. Common symptoms of neutropenia in patients include, for example, fever, mouth sores, and ear infections. Patients with profound neutropenia often suffer from pyogenic infections such as septicemia, cutaneous cellulitis, liver abscesses, furunculosis, pneumonia, stomatitis, gingivitis, perirectal inflammation, colitis, sinusitis, and otitis media.

In some embodiments, the Absolute Neutrophil Count (ANC) is used to define levels of neutropenia. The ANC can be calculated from components of the complete blood count. In some embodiments, severity of neutropenia is classified based on the absolute neutrophil count (ANC) measured in cells per microliter of blood: a) mild neutropenia (1000 to 1500 cells/mL); b) moderate neutropenia (grade 3; 500 to 1000 cells/mL); c) severe neutropenia (grade 4; <500 cells/mL). In some embodiments, neutropenia can be graded according to criteria set forth in Table 5. Subjects with severe neutropenia often have severe risk of infection.

TABLE 5
Neutropenia Grading

| Grade | ANC |
|---|---|
| Grade 1 | <2.0 × 10⁹/L (<2000/mm³) and >1.5 × 10⁹/L (>1500/mm³) |
| Grade 2 | <1.5 × 10⁹/L (<1500/mm³) and >1.0 × 10⁹/L (>1000/mm³) |
| Grade 3 | <1.0 × 10⁹/L (<1000/mm³) and >0.5 × 10⁹/L (>500/mm³) |
| Grade 4 | <0.5 × 10⁹/L (<500/mm³) |

In some cases, neutropenia is a febrile neutropenia (also called neutropenic fever or neutropenic sepsis). Febrile neutropenia occurs when a patient has a temperature greater than 38° C. and low levels of neutrophils or neutropenia. In some embodiments, febrile neutropenia can be graded according to criteria set forth in Table 6.

TABLE 6
Exemplary Grading Criteria for Febrile Neutropenia

| Grade | Description of symptoms |
|---|---|
| Grade 3 | ANC <1000/mm³ and a single temperature of >38.3 degrees C. |
| | (101 degrees F.) or a sustained temperature of >=38 degrees C. |
| | (100.4 degrees F.) for more than one hour |
| Grade 4 | life-threatening consequences and indicated urgent intervention |
| Grade 5 | death |

In some embodiments, a subject is monitored for thrombocytopenia. Thrombocytopenia is characterized by a platelet count of less than 150,000 cells per microliter (μL). Presentation of thrombocytopenia, particularly among patients with more severe grades, may include bleeding, ecchymosis, petechiae, purpura, and hypersplenism. Thrombocytopenia may be characterized as grade 1 thrombocytopenia (i.e., platelet count of 75,000 to 150,000/μL), grade 2 (i.e., platelet count of 50,000 to <75,000/μL), grade 3 (platelet count of 25,000 to <50,000/μL), or grade 4 (i.e., platelet count of below 25,000/μL).

In some embodiments of the provided methods, if a subject is determined to exhibit a hematological toxicity, such as thrombocytopenia and/or neutropenia or a particular grade thereof, the combination therapy with Compound C can be altered. In some aspects, the combination therapy is altered if, after administration of Compound C, the subject has a grade 3 or higher thrombocytopenia; a grade 3 neutropenia; a grade 3 neutropenia that is sustained (such as at least more than 3, 5, or 7 days); a grade 4 neutropenia; a Grade 3 or higher febrile neutropenia. In some embodiments, administration of Compound C is halted permanently or suspended until signs or symptoms of the toxicity is resolved, lessened or reduced. Continued monitoring of the subject can be carried out to assess one or more signs or symptoms of the toxicity, such as by CBC or differential leukocyte analysis. In some cases, if the toxicity resolves or is reduced, administration of Compound C can be restarted at the same dose or dosing regimen prior to suspending the combination therapy, at a lower or reduced dose, and/or in a dosing regimen involving less frequent dosing. In some embodiments, in instances of restarting the combination therapy, the dose is lowered or reduced at least or at least about or about 10%, 15%, 20%, 25%, 30%, 40%, 50%, or 60%. In some embodiments, if the dose prior to suspending the cell therapy is 0.3 mg, the dose is reduced to 0.2 mg. In some aspects, if a hematological toxicity is of such severity that suspension of the combination therapy is for greater than 4 weeks, the combination therapy can be permanently discontinued.

In some embodiments, one or more agents can be administered to the subject to treat, ameliorate or lessen one or more symptoms associated with the hematological toxicity. In some cases, a myeloid growth factors, such as G-CSF or GM-CSF, is administered to the subject until the hematological toxicity improves. Examples of such therapies include filgrastim or pegfilgrastim. In some aspects, such agents are administered to subjects experiencing severe neutropenia or febrile neutropenia, including any grade 3 or greater neutropenia of any duration.

### C. Non-Hematologic Toxicity

In some aspects, the toxic outcome is or is associated with or indicative of one or more non-hematologic toxicity following administration of Compound C. Examples of non-hematologic toxicities include, but are not limited to, tumor flare reaction, infections, tumor lysis syndrome, cardiac laboratory abnormalities, thromboembolic event(s) (such as deep vein thrombosis and pulmonary embolism), and/or pneumonitis.

In some aspects, the non-hematologic toxicity is tumor flare reaction (TFR) (sometimes also referred to pseudoprogression). TFR is a sudden increase in the size of the disease-bearing sites, including the lymph nodes, spleen and/or the liver often accompanied by a low-grade fever, tenderness and swelling, diffuse rash and in some cases, an increase in the peripheral blood lymphocyte counts. In some embodiments, TFR is graded according to Common Terminology Criteria for Adverse Events (Version 3.0; US National Cancer Institute, Bethesda, MD, USA). In some embodiments, TFR is graded as follows: grade 1, mild pain not interfering with function; grade 2, moderate pain, pain or analgesics interfering with function but not interfering with activities of daily living (ADL); grade 3, severe pain, pain or analgesics interfering with function and interfering with ADL; grade 4, disabling; grade 5, death. In some embodiments, one or more agents can be administered to the subject to treat, ameliorate or lessen one or more symptoms associated with TFR, such as corticosteroids, NSAIDs and/or narcotic analgesic.

In some aspects, the non-hematologic toxicity is tumor lysis syndrome (TLS). In some embodiments, TLS can be graded according to criteria specified by the Cairo-Bishop grading system (Cairo and Bishop (2004) Br J Haematol, 127:3-11). In some embodiments, subjects can be given intravenous hydration to reduce hyperuricemia.

In some embodiments, subjects can be monitored for cardiac toxicity, such as by monitoring ECGS, LVEF and monitoring levels of troponin-T and BNP. In some embodiments, a cardiac toxicity that potentially may necessitate holding or suspending Compound C may occur if elevated levels of troponin-T and/or BNP with one or more cardiac symptoms is observed.

In some embodiments of the provided methods, if a subject is determined to exhibit a non-hematological toxicity, such as TFR or other non-hematological toxicity or a particular grade thereof, the combination therapy with Compound C can be altered. In some aspects, the combination therapy is altered if, after administration of Compound C, the subject has a grade 3 or higher non-hematological toxicity, such as grade 3 or higher TFR. In some embodiments, administration of Compound C is halted permanently or suspended until signs or symptoms of the toxicity is resolved, lessened or reduced. Continued monitoring of the subject can be carried out to assess one or more signs or symptoms of the toxicity. In some cases, if the toxicity resolves or is reduced, administration of Compound C can be restarted at the same dose or dosing regimen prior to suspending the combination therapy, at a lower or reduced dose, and/or in a dosing regimen involving less frequent dosing. In some embodiments, in instances of restarting the combination therapy, the dose is lowered or reduced at least or at least about or about 10%, 15%, 20%, 25%, 30%, 40%, 50%, or 60%. In some embodiments, if the dose prior to suspending the cell therapy is 0.3 mg, the dose is reduced to 0.2 mg. In some embodiments, if a grade 3 toxicity recurs even after a dose reduction, the dose can be further reduced. In some embodiments, if a grade 4 toxicity recurs even after a dose reduction, the combination therapy can be permanently discontinued. In some aspects, if a hematological toxicity is of such severity that suspension of the combination therapy is for greater than 4 weeks, the combination therapy can be permanently discontinued.

### V. ARTICLES OF MANUFACTURE AND KITS

Also provided are articles of manufacture containing Compound C, and components for the immunotherapy, e.g., antibody or antigen binding fragment thereof or T cell therapy, e.g. engineered cells, and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition includes the engineered cells used for the immunotherapy, e.g. T cell therapy; and (b) a second container with a composition contained therein, wherein the composition includes Compound C.

In some embodiments, the first container comprises a first composition and a second composition, wherein the first composition comprises a first population of the engineered cells used for the immunotherapy, e.g., CD4+ T cell therapy, and the second composition comprises a second population of the engineered cells, wherein the second population may be engineered separately from the first population, e.g., CD8+ T cell therapy. In some embodiments, the first and second cell compositions contain a defined ratio of the engineered cells, e.g., CD4+ and CD8+ cells (e.g., 1:1 ratio of CD4+:CD8+ CAR+ T cells).

The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, a “subject” is a mammal, such as a human or other animal, and typically is human. In some embodiments, the subject, e.g., patient, to whom Compound C, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as B cell malignancy). This delay can be of varying lengths of time, depending on the history of the disease and/or individual being treated. As is evident, a sufficient or significant delay can, in effect, encompass prevention, in that the individual does not develop the disease. For example, a late stage B cell lymphoma, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., engineered cells or anti-PD-L1 or antigen-binding fragment, or a pharmaceutical formulation or composition thereof, in the context of administration, refers to an amount effective, at dosages/amounts and for periods of time necessary, to achieve a desired result, such as a therapeutic or prophylactic result.

A “therapeutically effective amount” of an agent, e.g., engineered cells or anti-PD-L1 or antigen-binding fragment, or a pharmaceutical formulation or composition thereof, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering Compound C, engineered cells (e.g. cell therapy), or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compound targeting Cereblons, including cells. It may be a solution, a suspension, liquid, powder, a paste, aqueous, non-aqueous or any combination thereof.

As used herein, a “subject” is a mammal, such as a human or other animal, and typically is human.

### VII. EXEMPLARY EMBODIMENTS

Among the provided embodiments are:
1. A method of treating a lymphoma, the method comprising administering to a subject having a lymphoma a combination therapy comprising:
(i) a cell therapy comprising a dose of engineered cells comprising T cells expressing a chimeric antigen receptor (CAR) that binds cluster of differentiation 19 (CD19), wherein the cell therapy is administered on Day 1 of the combination therapy; and
(ii) a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

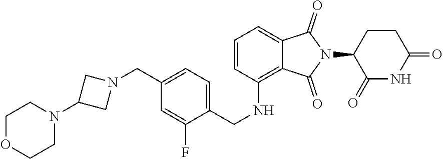 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein the compound is administered as a plurality of intermittent doses administered no more than once weekly, wherein each dose is between at or about 0.1 mg and at or about 0.6 mg, inclusive, wherein the administration of the compound is initiated between Day 1 and Day 29, inclusive, of the combination therapy.
2. A method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

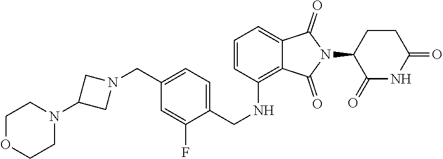 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered no more than once weekly, wherein each dose is between at or about 0.1 mg and at or about 0.6 mg, inclusive, and wherein the administration of the compound is initiated between Day 1 and Day 29, inclusive, of the combination therapy.
3 The method of embodiment 1 or embodiment 2, wherein the administration of the compound is initiated between Day 1 and Day 22, inclusive.
4. The method of embodiment 1 or embodiment 2, wherein the administration of the compound is initiated between Day 1 and Day 15, inclusive.
5. The method of any of embodiments 1-4, wherein the administration of the compound is initiated between Day 8 and Day 15, inclusive.
6. The method of any of embodiments 1-4, wherein the administration of the compound is initiated at or about Day 1.
7. The method of any of embodiments 1-5, wherein the administration of the compound is initiated at or about Day 8.
8. The method of any of embodiments 1-5, wherein the administration of the compound is initiated at or about Day 15.
9. The method of any of embodiments 1-8, wherein each dose of the plurality of intermittent doses is the same.
10. The method of any of embodiments 1-9, wherein the compound is administered once weekly.
11. The method of any of embodiments 1-10, wherein the compound is administered once every 7 days (Q7D).
12. The method of any of embodiments 1-9, wherein the compound is administered once every two weeks.
13. The method of any of embodiments 1-9 and 12, wherein the compound is administered once every 14 days (Q14D).
14. The method of any of embodiments 1-13, wherein the compound is administered for at least 12 weeks after the administration of the cell therapy.
15. The method of any of embodiments 1-13, wherein the compound is administered for up to 12 weeks after the administration of the cell therapy.
16. The method of any of embodiments 1-4, 6, 9-11, 14, and 15, wherein the compound is administered on Days 1, 8, 15, 22, 29, 36, 43, 50, 57, 64, 71, 78, and 85.
17. The method of any of embodiments 1-5, 7, 9-11, 14, and 15, wherein the compound is administered on Days 8, 15, 22, 29, 36, 43, 50, 57, 64, 71, 78, and 85.
18. The method of any of embodiments 1-5, 8-11, 14, and 15, wherein the compound is administered on Days 15, 22, 29, 36, 43, 50, 57, 64, 71, 78, and 85.
19. The method of any of embodiments 1-5, 7, 9, and 12-15, wherein the compound is administered on Days 8, 22, 36, 50, 64, and 78.
20. The method of any of embodiments 1-19, wherein the dose of the compound is between at or about 0.2 mg and at or about 0.4 mg, inclusive.
21. The method of any of embodiments 1-20, wherein the dose of the compound is at or about 0.4 mg.
22. The method of any of embodiments 1-20, wherein the dose of the compound is less than 0.4 mg.
23. The method of any of embodiments 1-20 and 22, wherein the dose of the compound is at or about 0.3 mg.
24. The method of any of embodiments 1-20 and 22, wherein the dose of the compound is at or about 0.2 mg.
25. A method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

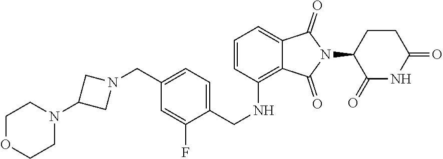 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 8, 15, 22, 29, 36, 43, 50, 57, 64, 71, 78, and 85, wherein each dose is 0.3 mg.
26. A method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

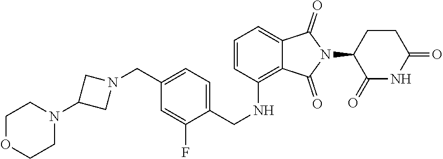 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 1, 8, 15, 22, 29, 36, 43, 50, 57, 64, 71, 78, and 85, wherein each dose is 0.3 mg.
27. A method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

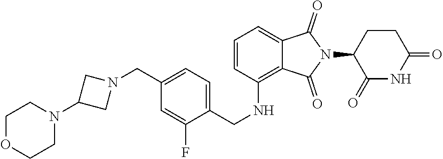 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 15, 22, 29, 36, 43, 50, 57, 64, 71, 78, and 85, wherein each dose is 0.3 mg.
28. A method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

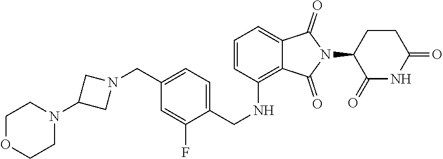 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 8, 15, 22, 29, 36, 43, 50, 57, 64, 71, 78, and 85, wherein each dose is 0.4 mg.
29. A method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

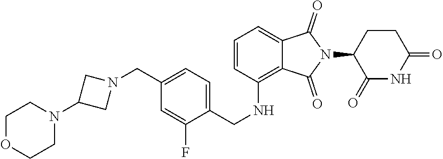 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every 14 days (Q14D) and is administered on Days 8, 22, 36, 50, 64, and 78, wherein each dose is 0.3 mg.
30. A method of treating a lymphoma, the method comprising administering to a subject having a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione having the following structure:

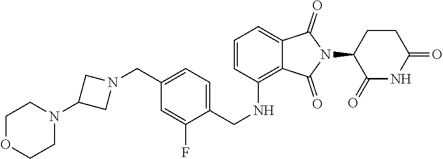 [SMILES: O=C1CC[C@H](N2C(=O)c3cccc(NCc4ccc(CN5CC(N6CCOCC6)C5)cc4F)c3C2=O)C(=O)N1]

or a pharmaceutically acceptable salt, solvate, hydrate, co-crystal, clathrate, or polymorph thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 8, 15, 22, 29, 36, 43, 50, 57, 64, 71, 78, and 85, wherein each dose is 0.2 mg.
31. The method of any of embodiments 1-30, wherein the compound is or comprises a pharmaceutically acceptable salt of (S)-2-(2,6-dioxopiperidin-3-yl)-4-((2-fluoro-4-((3-morpholinoazetidin-1-yl)methyl)benzyl)amino)isoindoline-1,3-dione.
32 The method of any of embodiments 1-30, wherein the compound is or comprises a hydrate of (S)-2-(2,6-dioxopiperidin-3-yl)-4-((2-fluoro-4-((3-morpholinoazetidin-1-yl)methyl)benzyl)amino)isoindoline-1,3-dione.
33. The method of any of embodiments 1-30, wherein the compound is or comprises a solvate of (S)-2-(2,6-dioxopiperidin-3-yl)-4-((2-fluoro-4-((3-morpholinoazetidin-1-yl)methyl)benzyl)amino)isoindoline-1,3-dione.
34. The method of any of embodiments 1-30, wherein the compound is or comprises (S)-2-(2,6-dioxopiperidin-3-yl)-4-((2-fluoro-4-((3-morpholinoazetidin-1-yl)methyl)benzyl)amino)isoindoline-1,3-dione.
35. The method of any of embodiments 1-34, wherein at the time of the initiation of the administration of the compound, the subject does not exhibit a severe toxicity following the administration of the cell therapy.
36. The method of embodiment 35, wherein:
the severe toxicity is severe cytokine release syndrome (CRS), optionally grade 3 or higher CRS, prolonged grade 3 or higher CRS, grade 4 CRS, or grade 5 CRS; and/or the severe toxicity is severe neurotoxicity, optionally grade 3 or higher neurotoxicity, prolonged grade 3 or higher neurotoxicity, grade 4 neurotoxicity, or grade 5 neurotoxicity.
37. The method of any one of embodiments 1-36, wherein the administration of the compound is suspended and/or the dose of the compound is modified, optionally lowered, if the subject exhibits a toxicity following the administration of the compound, optionally a hematologic toxicity.
38. The method of embodiment 37, wherein the toxicity is severe thrombocytopenia, optionally grade 4 thrombocytopenia or prolonged grade 4 thrombocytopenia.
39. The method of embodiment 37, wherein the toxicity is severe neutropenia, optionally grade 4 neutropenia, prolonged grade 4 neutropenia, or febrile neutropenia, optionally grade 3 or higher febrile neutropenia or prolonged grade 3 or higher febrile neutropenia.
40. The method of any of embodiments 37-39, wherein the administration of the compound is restarted after the subject no longer exhibits the toxicity.
41. The method of any one of embodiments 1-40, wherein the lymphoma is a B-cell malignancy.
42. The method of any of embodiments 1-41, wherein the lymphoma is a non-Hodgkin lymphoma (NHL), optionally wherein the NHL comprises aggressive NHL; diffuse large B cell lymphoma (DLBCL); DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally follicular lymphoma Grade 3B (FL3B); and/or high-grade B-cell lymphoma with MYC and BCL2 and/or BCL6 rearrangements with DLBCL histology (double/triple hit).
43. The method of any of embodiments 1-42, wherein the CD19 is a human CD19.
44. The method of any of embodiments 1-43, wherein the chimeric antigen receptor (CAR) comprises an extracellular antigen-recognition domain that specifically binds to the CD19 and an intracellular signaling domain comprising an ITAM.
45. The method of embodiment 44, wherein the intracellular signaling domain comprises a signaling domain of a CD3-zeta (CD3) chain, optionally a human CD3-zeta chain. 46. The method of embodiment 44 or embodiment 45, wherein the chimeric antigen receptor (CAR) further comprises a costimulatory signaling region.
47. The method of embodiment 46, wherein the costimulatory signaling region comprises a signaling domain of CD28 or 4-1BB, optionally human CD28 or human 4-1BB.
48. The method of embodiment 46 or embodiment 47, wherein the costimulatory signaling region comprises a signaling domain of human 4-1BB.
49. The method of any of embodiments 1-48, wherein:
the CAR comprises an scFv specific for the CD19; a transmembrane domain; a cytoplasmic signaling domain derived from a costimulatory molecule, which optionally is or comprises a 4-1BB, optionally human 4-1BB; and a cytoplasmic signaling domain derived from a primary signaling ITAM-containing molecule, which optionally is or comprises a CD3zeta signaling domain, optionally a human CD3zeta signaling domain; and optionally wherein the CAR further comprises a spacer between the transmembrane domain and the scFv.
50. The method of any of embodiments 1-48, wherein:
the CAR comprises, in order, an scFv specific for the CD19; a transmembrane domain; a cytoplasmic signaling domain derived from a costimulatory molecule, which optionally is or comprises a 4-1BB signaling domain, optionally a human 4-1BB signaling domain; and a cytoplasmic signaling domain derived from a primary signaling ITAM-containing molecule, which optionally is a CD3zeta signaling domain, optionally human CD3zeta signaling domain.
51. The method of any of embodiments 1-48, wherein the CAR comprises, in order, an scFv specific for the CD19; a spacer; a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.
52. The method of any of embodiments 49-51, wherein the spacer is a polypeptide spacer that comprises or consists of all or a portion of an immunoglobulin hinge or a modified version thereof or comprises about 15 amino acids or less.
53. The method of any of embodiments 49-52, wherein the spacer comprises or consists of all or a portion of an immunoglobulin hinge, optionally an IgG4 hinge, or a modified version thereof and/or comprises about 15 amino acids or less.
54. The method of embodiment 52 or embodiment 53, wherein the spacer is at or about 12 amino acids in length and/or comprises or consists of all or a portion of an immunoglobulin hinge, optionally an IgG4, or a modified version thereof
55. The method of any of embodiments 49-54, wherein the spacer has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto.
56. The method of any of embodiments 49-55, wherein the cytoplasmic signaling domain derived from a costimulatory molecule comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto.
57. The method of any of embodiments 49-56, wherein the cytoplasmic signaling domain derived from a primary signaling ITAM-containing molecule comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto.
58. The method of any of embodiments 49-57, wherein the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40).
59. The method of any of embodiments 49-58, wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 and optionally wherein the scFv comprises a VH comprising SEQ ID NO: 41, and a VL comprising the amino acid sequence set forth as SEQ ID NO: 42.
60. The method of any of embodiments 49-58, wherein the scFv has the sequence of amino acids set forth in SEQ ID NO: 43.
61. The method of any of embodiments 1-60, wherein the dose of engineered cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, or 5×10⁷ to 1×10⁸ total CAR-expressing T cells, each inclusive.
62. The method of any of embodiments 1-61, wherein the dose of engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.
63. The method of any of embodiments 1-62, wherein the dose of engineered cells comprises at or about 1×10⁸ CAR-expressing T cells.
64. The method of any of embodiments 1-63, wherein the dose of engineered cells is administered parenterally, optionally intravenously.
65. The method of any of embodiments 1-64, wherein the T cells are primary T cells obtained from the subject.
66. The method of any of embodiments 1-65, wherein the T cells are autologous to the subject.
67. The method of any of embodiments 1-64, wherein the T cells are allogeneic to the subject.
68. The method of any of embodiments 1-67, wherein the dose of engineered cells comprises CD4+ T cells expressing the CAR and CD8+ T cells expressing the CAR and the administration of the dose comprises administering a plurality of separate compositions, said plurality of separate compositions comprising a first composition comprising one of the CD4+ T cells and the CD8+ T cells and a second composition comprising the other of the CD4+ T cells or the CD8+ T cells.
69. The method of embodiment 68, wherein:
the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or
the initiation of administration of the first composition and the initiation of administration of the second composition are carried out between about 1 minute and about 1 hour apart or between about 5 minutes and about 30 minutes apart.
70. The method of embodiment 68 or embodiment 69, wherein the first composition and second composition are administered no more than 2 hours, no more than 1 hour, no more than 30 minutes, no more than 15 minutes, no more than 10 minutes or no more than 5 minutes apart.
71. The method of any of embodiments 68-70, wherein the first composition comprises the CD4+ T cells.
72. The method of any of embodiments 68-70, wherein the first composition comprises the CD8+ T cells.
73. The method of any of embodiments 68-72, wherein the first composition is administered prior to the second composition.
74. The method of any one of embodiments 1-73, wherein, prior to the administration of the cell therapy, the subject has been preconditioned with a lymphodepleting therapy comprising the administration of fludarabine and/or cyclophosphamide.
75. The method of any one of embodiments 1-73, further comprising, immediately prior to the administration of the cell therapy, administering a lymphodepleting therapy to the subject comprising the administration of fludarabine and/or cyclophosphamide.
76. The method of embodiment 74 or embodiment 75, wherein the lymphodepleting therapy comprises administration of cyclophosphamide at about 200-400 mg/m², optionally at or about 300 mg/m², inclusive, and/or fludarabine at about 20-40 mg/m², optionally 30 mg/m², daily for 2-4 days, optionally for 3 days, or wherein the lymphodepleting therapy comprises administration of cyclophosphamide at about 500 mg/m².
77. The method of any one of embodiments 74-76, wherein:
the lymphodepleting therapy comprises administration of cyclophosphamide at or about 300 mg/m² and fludarabine at about 30 mg/m² daily for 3 days; and/or
the lymphodepleting therapy comprises administration of cyclophosphamide at or about 500 mg/m² and fludarabine at about 30 mg/m² daily for 3 days.
78. The method of any one of embodiments 1-77, wherein the subject is a human.
79. The method of any of embodiments 1-78, wherein:
at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or
wherein at least 60, 70, 80, 90, or 95% of subjects achieving a CR by six months remain in response, remain in CR, and/or survive or survive without progression, for greater at or greater than 3 months and/or at or greater than 6 months and/or at greater than nine months; and/or
at least 50%, at least 60% or at least 70% of the subjects treated according to the method achieve objective response (OR) optionally wherein the OR is durable, or is durable in at least 60, 70, 80, 90, or 95% of subjects achieving the OR, for at or greater than 6 months or at or greater than 9 months; and/or
wherein at least 60, 70, 80, 90, or 95% of subjects achieving an OR by six months remain in response or surviving for greater at or greater than 3 months and/or at or greater than 6 months.
80. The method of any of embodiments 42-79, wherein, at or immediately prior to the time of the administration of the dose of engineered cells, the subject has relapsed following remission after treatment with, or become refractory to, one or more prior therapies for the NHL, optionally one, two, or three prior therapies other than another dose of engineered cells expressing the CAR.
81. The method of any of embodiment 42-80, wherein, at or prior to the administration of the dose of engineered cells:
the subject is or has been identified as having a double/triple hit lymphoma;
the subject is or has been identified as having a chemorefractory lymphoma, optionally a chemorefractory DLBCL; and/or
the subject has not achieved complete remission (CR) in response to a prior therapy.
82. The method of any of embodiments 1-81, wherein the administration of the compound:
reverses an exhaustion phenotype in CAR-expressing T cells in the subject;
prevents, inhibits, or delays the onset of an exhaustion phenotype in CAR-expressing T cells in the subject;
reduces the level or degree of an exhaustion phenotype in CAR-expressing T cells in the subject; or
reduces the percentage or the total number of CAR-expressing T cells in the subject that have an exhaustion phenotype.
83. The method of any of embodiments 1-82, wherein the initiation of the administration of the compound is carried out subsequently to the administration of the cell therapy and, following administration of the compound or initiation thereof, the subject exhibits a restoration or rescue of an antigen- or tumor-specific activity or function of the CAR-expressing T cells in said subject, optionally wherein said restoration, rescue, and/or initiation of administration of said compound, is at a point in time after CAR-expressing T cells in the subject or in the blood of the subject have exhibited an exhausted phenotype.
84. The method of any of embodiments 1-83, wherein the administration of the compound comprises administration at an amount, frequency and/or duration effective to:
(a) effect an increase in antigen-specific or antigen receptor-driven activity of naïve or non-exhausted T cells in the subject, which optionally comprise T cells expressing said CAR, following exposure of the T cells to CD19 antigen or to an antigen receptor-specific agent compared to the absence of said administration of said compound; or
(b) prevent, inhibit or delay the onset of an exhaustion phenotype, in naïve or non-exhausted T cells in the subject, which optionally comprise T cells expressing said CAR, following exposure of the T cells to CD19 antigen or to an antigen receptor-specific agent, as compared to the absence of said administration of said compound; or
(c) reverse an exhaustion phenotype in exhausted T cells, optionally comprising T cells expressing said CAR, in the subject, as compared to the absence of said administration of said subject.
85. The method of embodiment 84, wherein the administration of the compound comprises administration at an amount, frequency and/or duration effective (i) to effect said increase in activity and (ii) to prevent, inhibit or delay said onset of said exhaustion phenotype and/or reverse said exhaustion phenotype.
86. The method of embodiments 84 or embodiment 85, wherein the T cells in the subject comprise T cells expressing said CAR and/or said antigen is CD19.
87. The method of any of embodiments 82-86, wherein the exhaustion phenotype, with reference to a T cell or population of T cells, comprises:
an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or
a decrease in the level or degree of an activity exhibited by said T cells or population of T cells upon exposure to a CD19 antigen or antigen receptor-specific agent, compared to a reference T cell population, under the same conditions.
88. The method of embodiment 87, wherein the increase in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.
89. The method of embodiment 87, wherein the decrease in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.
90. The method of any of embodiments 87-89, wherein the reference T cell population is a population of T cells known to have a non-exhausted phenotype, is a population of naïve T cells, is a population of central memory T cells, or is a population of stem central memory T cells, optionally from the same subject, or of the same species as the subject, from which the T cell or T cells having the exhaustion phenotype are derived.
91. The method of any of embodiments 87-90, wherein the reference T cell population (a) is a subject-matched population comprising bulk T cells isolated from the blood of the subject from which the T cell or T cells having the exhaustion phenotype is derived, optionally wherein the bulk T cells do not express the CAR and/or (b) is obtained from the subject from which the T cell or T cells having the exhaustion phenotype is derived, prior to receiving administration of a dose of T cells expressing the CAR.
92. The method of any of embodiments 87-91, wherein the reference T cell population is a composition comprising a sample of the T cell therapy, or pharmaceutical composition comprising T cells expressing the CAR, prior to its administration to the subject, optionally wherein the composition is a cryopreserved sample.
93. The method of any of embodiments 87-92, wherein the one or more exhaustion marker is an inhibitory receptor.
94. The method of any of embodiments 87-93, wherein the one or more exhaustion marker is selected from among PD-1, CTLA-4, TIM-3, LAG-3, BTLA, 2B4, CD160, CD39, VISTA, and TIGIT.
95. The method of any of embodiments 87-94, wherein the activity or is one or more of proliferation, cytotoxicity or production of one or a combination of inflammatory cytokines, optionally wherein the one or a combination of cytokines is selected from the group consisting of IL-2, IFN-gamma and TNF-alpha.
96. The method of any of embodiments 87-95,
wherein the exposure to said CD19 antigen or antigen receptor-specific agent comprises incubation with the CD19 antigen or antigen receptor-specific agent, optionally an agent that binds the antigen-binding domain of the CAR.
97. The method of embodiment 96, wherein the exposure to the CD19 antigen or antigen receptor-specific agent comprises exposing the T cells to CD19 antigen-expressing target cells, optionally cells of the B cell malignancy.

### VIII. EXAMPLES

The following examples are included for illustrative purposes only and are not intended to limit the scope of the invention.

### Example 1: Degradation of Aiolos and Ikaros Transcription Factors in Anti-CD19 CAR-T Cells by Compound C

The ability of (S)-2-(2,6-dioxopiperidin-3-yl)-4-((2-fluoro-4-((3-morpholinoazetidin-1-yl)methyl)benzyl)amino)isoindoline-1,3-dione (Compound C) to degrade Aiolos and Ikaros transcription factors in stimulated anti-CD19 CAR T cells was assessed.

T cell compositions containing anti-CD19 CAR-expressing T cells were generated from leukapheresis samples from five healthy human adult donors by a process that included immunoaffinity-based selection of T cells by separately selecting CD4+ and CD8+ cells from the samples, resulting in two compositions enriched for CD4+ and CD8+ cells, respectively, per donor. Cells of the enriched CD4+ and CD8+ compositions were separately activated with anti-CD3/anti-CD28 beads and subjected to lentiviral transduction with a vector encoding an anti-CD19 CAR. The anti-CD19 CAR contained an anti-CD19 single-chain variable fragment (scFv) derived from a murine antibody (variable region derived from FMC63), an immunoglobulin-derived spacer, a transmembrane domain derived from CD28, a costimulatory region derived from 4-1BB, and a CD3-zeta intracellular signaling domain. The expression construct in the viral vector further contained sequences encoding a surrogate surface marker for CAR expression, which was separated from the CAR-encoding sequence by a T2A ribosome skip sequence. Transduced populations then were separately incubated in the presence of stimulating reagents for cell expansion. Expanded CD4+ and CD8+ cells were formulated and cryopreserved separately and stored.

Prior to use, the cryopreserved CD4+ and CD8+ engineered cell compositions were thawed and combined at approximately a 1:1 CD4:CD8 ratio for each donor. Cells of the combined composition were then stimulated at 37° C. with 5% CO₂ for 24 hours with 30 g/mL of a CAR-specific anti-idiotypic antibody (see, e.g., WO2018/023100). This stimulation took place in the presence of a vehicle control or in the presence of Compound C; (S)-3-[4-(4-morpholin-4-ylmethyl-benzyloxy)-1-oxo-1,3-dihydro-isoindol-2-yl]-piperidine-2,6-dione (Compound 2); or 3-(5-amino-2-methyl-4-oxo-4H-quinazolin-3-yl)-piperidine-2,6-dione (Compound 1) at concentrations ranging from approximately 10⁻¹ to 10⁴ nM.

Following incubation, the stimulated cells were stained with antibodies and analyzed by flow cytometry to assess intracellular levels of Ikaros and Aiolos in CAR+ T cells, as measured by median fluorescence intensity (MFI). MFI values for Ikaros and Aiolos were normalized and calculated as a percentage relative to vehicle control.

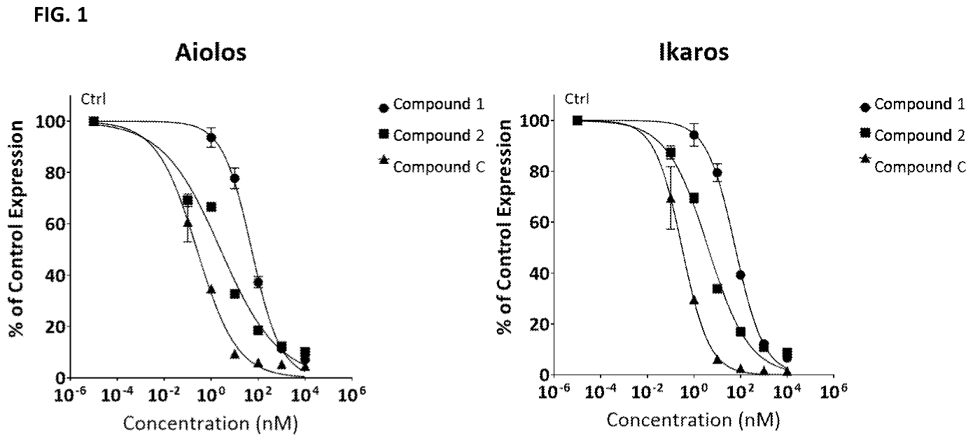

Concentration-dependent decreases in intracellular Ikaros and Aiolos expression were observed in the CAR-expressing stimulated T cells after incubation with Compound C, Compound 2, and Compound 1 (FIG. 1). At all compound concentration levels tested, Ikaros and Aiolos expression were lower in cells treated with Compound C than with Compound 2 or Compound 1. In addition, the EC50 values for reducing Aiolos and Ikaros expression for Compound C, Compound 2, and Compound 1 were calculated as the compound concentrations that reduced Aiolos and Ikaros MFIs to 50% of their MFIs in the absence of the compounds. These EC50 values are shown in Table E1 as averages (the 95% confidence intervals across the five donors are shown in parentheses). The results show that Compound C is a more potent Ikaros and Aiolos degrader in anti-CD19 CAR T cells than is Compound 2 or Compound 1.

TABLE E1
Average EC50 Values (95% Confidence Interval)

| | Compound C | Compound 2 | Compound 1 |
|---|---|---|---|
| Aiolos | 0.25 (0.17-0.34) | 2.50 (1.55-3.90) | 52.66 (41.7-66.54) |
| Ikaros | 0.31 (0.23-0.40) | 4.02 (2.98-5.40) | 59.33 (47.56-74.06) |

### Example 2: Effects of Compound Con CAR-T Cell Function

T cell function of anti-CD19 CAR T cells treated with Compound C was assessed.

Anti-CD19 CAR-expressing T cell compositions were generated from three different healthy donors as described in Example 1. Prior to use, cryopreserved CD4+ and CD8+ engineered cell compositions were thawed and combined at approximately a 1:1 CD4:CD8 ratio.

### A. Incubation With Anti-Idiotypic Antibody and Compound C

To assess viability, proliferation, cell cycle phase, and cytokine production, the combined CD4+ and CD8+ CAR-expressing T cell compositions were incubated with plate-bound CAR-specific anti-idiotypic antibody (see, e.g., WO2018/023100) and incubated at 37° C. in the presence of Compound C, Compound 2, or Compound 1. Following incubation, stimulated CAR T cells were harvested, washed twice in media, and assessed.

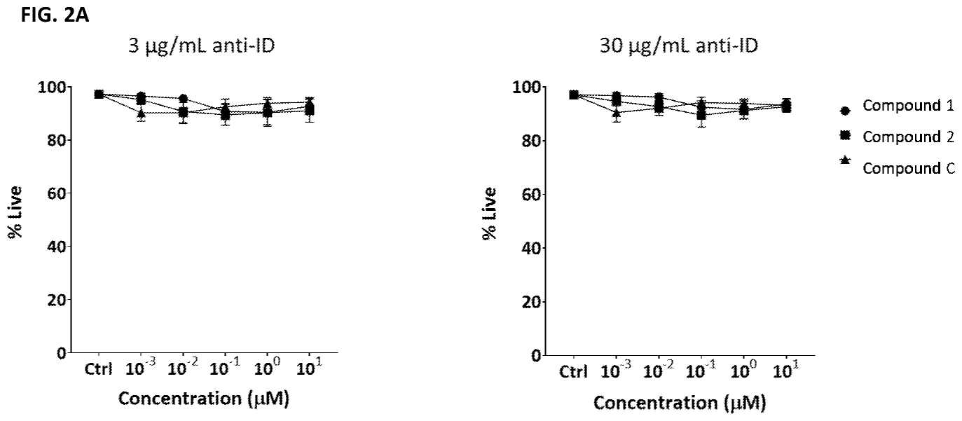

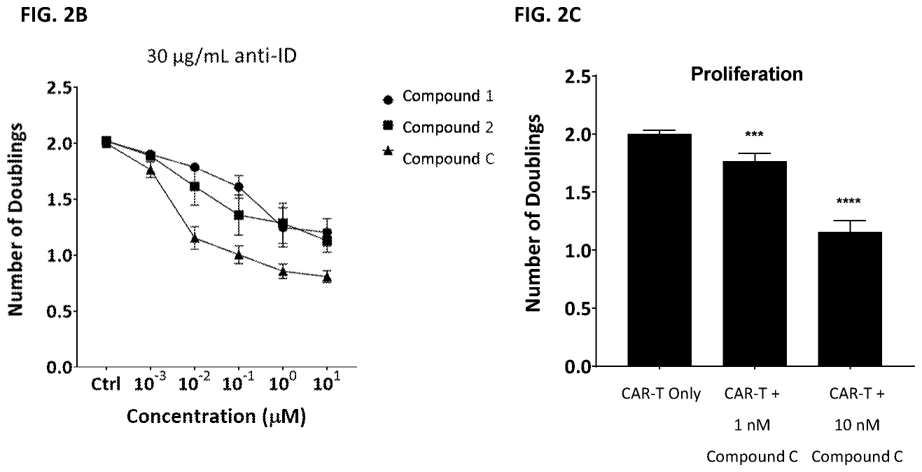

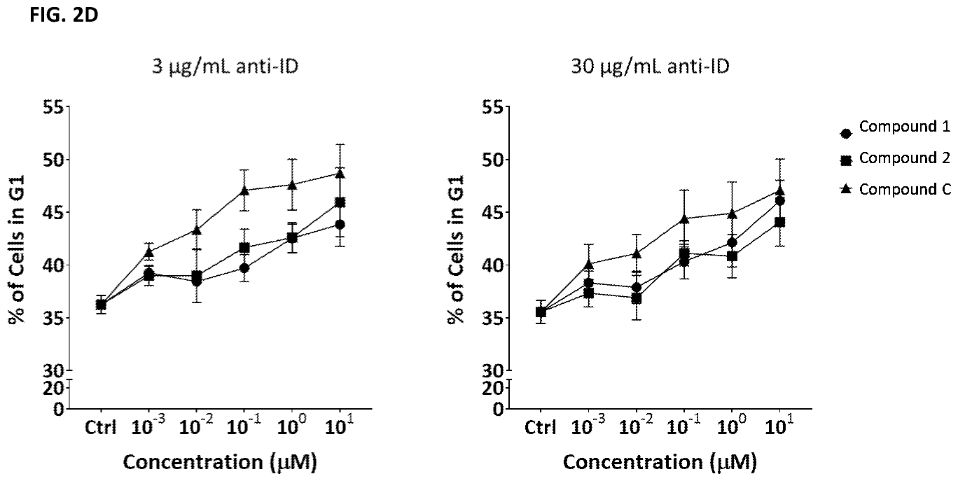

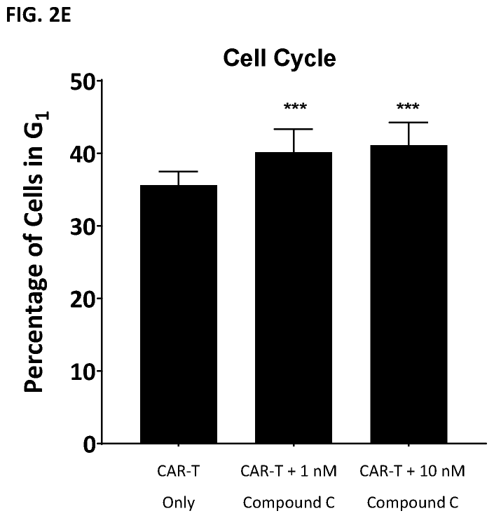

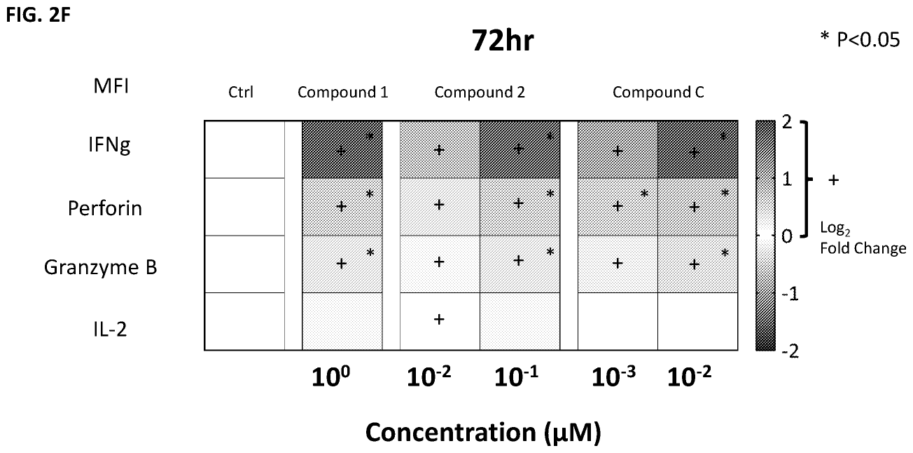

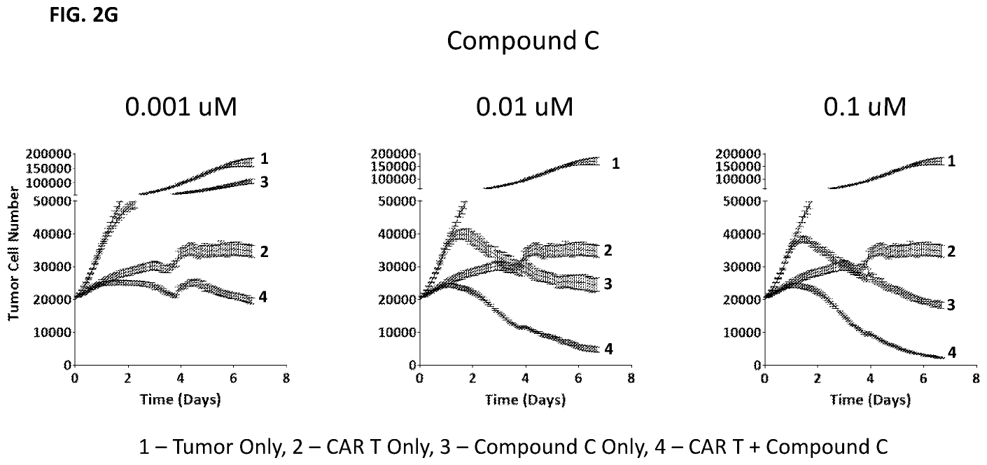

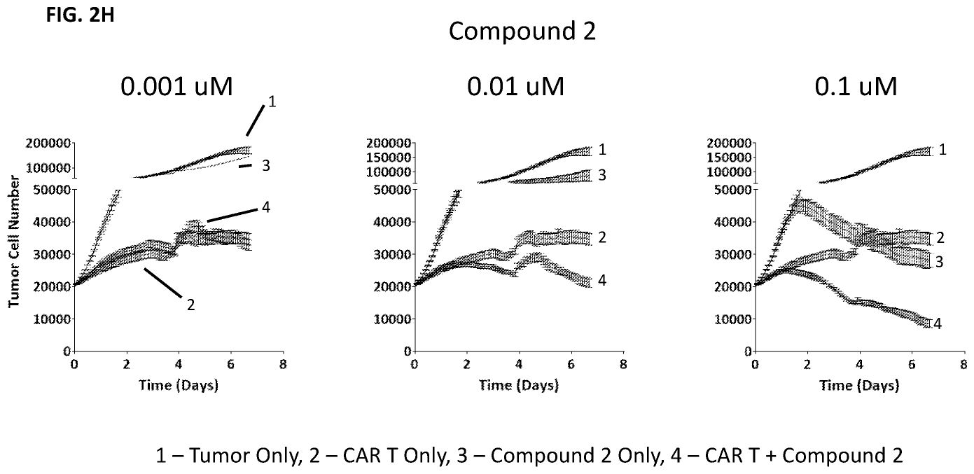

Viability was assessed at Day 3 of culture by flow cytometry using a live/dead cell dye. As shown in FIG. 2A for the three donors (mean±SEM), the compounds had no effect on cell viability when stimulation was carried out either with 3 μg/mL of anti-idiotypic antibody (left panel) or with 30 μg/mL of anti-idiotypic antibody (right panel).

Proliferation of T cells during culture with the plate-bound anti-idiotypic antibody was monitored for seven days using an IncuCyte® Live Cell Analysis System (Essen BioScience Inc.), and the number of cell doublings of cells stimulated with 30 μg/mL of anti-idiotypic antibody is shown in FIG. 2B and FIG. 2C for the three donors (mean±SEM). As shown in FIG. 2B, the presence of the compounds during CAR-specific stimulation with the anti-idiotypic antibody decreased the amount of CAR T cell proliferation. Cells treated with Compound C had the greatest decrease of doublings at all doses tested. As shown in FIG. 2C, proliferation was decreased in the presence of 1 nM of Compound C (*** P<0.001) and further decreased in the presence of 10 nM of Compound C (**** P<0.0001).

The cell cycle phases of CAR T cells treated with the compounds during CAR-specific stimulation with the plate-bound anti-idiotypic antibody were determined using EDU incorporation for two hours after three days of stimulation. For EDU incorporation, CAR T cells were labeled using the Click-iT™ EdU Cell Proliferation Kit (Thermo Fisher Scientific), after which the prepared cells were analyzed by flow cytometry. The results in FIG. 2D (left panel: stimulation with 3 μg/mL of anti-idiotypic antibody; right panel: stimulation with 30 μg/mL of anti-idiotypic antibody) show that treatment with higher doses of the compounds increased the percentage of CAR T cells in G1 phase of the cell cycle. Compound C had the most effect on the percentage of cells in G1 phase at all doses tested, with similar effects on percentage of cells in G1 phase of cell cycle at ten- or hundred-fold lower dose compared to the other compounds tested. As shown in FIG. 2E, the percentage of cells in G1 phase was increased in the presence of both 1 nM and 10 nM of Compound C (*** P<0.001). Without wishing to be bound by theory, the accumulation of CAR T cells in G1 phase may account for one of the mechanisms by which the compounds can limit onset of exhaustion during chronic stimulation, as shown in Example 3 below.

Intracellular cytokine levels in CAR T cells that had been stimulated for 72 hours with 30 μg/mL of anti-idiotypic antibody were determined. To do so, supernatants were collected from the stimulated CAR T cells and were analyzed for interferon-γ (IFN-γ) and interleukin-2 (IL-2) content using a multiplex electrochemiluminescence assay. To stain for intracellular cytokines, including Perforin and Granzyme B, cells were fixed and permeabilized using a Foxp3/Transcription Factor Staining Buffer Set (Thermo Fisher Scientific). Intracellular cytokine levels of IFNγ, perforin, granzyme B, and IL-2 were then determined by flow cytometry. As shown in FIG. 2F, treatment of cells with the compounds increased intracellular expression of effector cytokines (IFNγ, perforin, granzyme B), as determined by fold change in MFI compared to stimulation with the anti-idiotypic antibody in the presence of vehicle control, but had no significant effect on IL-2 production. Log₂ fold-change values greater than zero are indicated with a “+” in FIG. 2F. Similar results were observed when measuring the percentage of cells positive for the cytokines.

### B. Incubation With CD19+ Tumor Cells and Compound C

To assess effects of Compound C on cytolytic function of CAR T cells, the anti-CD19 CAR T cells were co-cultured with CAR T cell-resistant, CELMOD-sensitive RL CD19+ tumor cells at an effector to target (E:T) ratio of 1:1 and in the presence of varying concentrations of Compound C or Compound 2. To do so, CAR T cells were plated on ploy-D-lysine plates. CD19+ tumor cells transduced with IncuCyte® NucLight Red (Essen BioScience Inc.) were then added to the CAR T cells in addition to Compound C or vehicle control. Tumor cell number was measured over multiple days.

FIGS. 2G and 2H show cell number of RL tumor cells treated with anti-CD19 CAR T cells and Compound C or Compound 2, respectively (left panel: compound at 0.001 μM; middle panel: compound at 0.01 μM; right panel: compound at 0.1 μM). Treatment with CAR T cells in combination with either immunomodulatory compound led to lower tumor cell numbers than did CAR T cells alone or the compounds alone. As shown in FIG. 2G, tumor cell numbers were lower for all concentration levels at which Compound C was provided, including at 0.001 μM (P<0.0001) and at 0.01 μM (P<0.0001). As shown in FIG. 2H, tumor cell numbers were lower with Compound 2 at concentration levels of 0.01 μM and 0.1 μM, but not 0.001 μM.

### C. Summary

Together, these results show that without affecting CAR T cell viability, treatment with Compound C reduced CAR T cell proliferation, increased the number of cells in the G1 phase, and increased production of certain cytokines. Moreover, effects on CAR T cell proliferation and cell cycle were more pronounced for Compound C relative to Compound 2 or Compound 1, and cytolysis was improved by lower doses of Compound C relative to Compound 2.

### Example 3: Assessment of Chronically Stimulated CAR-T Cells Concurrently Treated with Compound C

The ability of Compound C to block or reduce exhaustion in chronically stimulated CAR T cells was assessed.

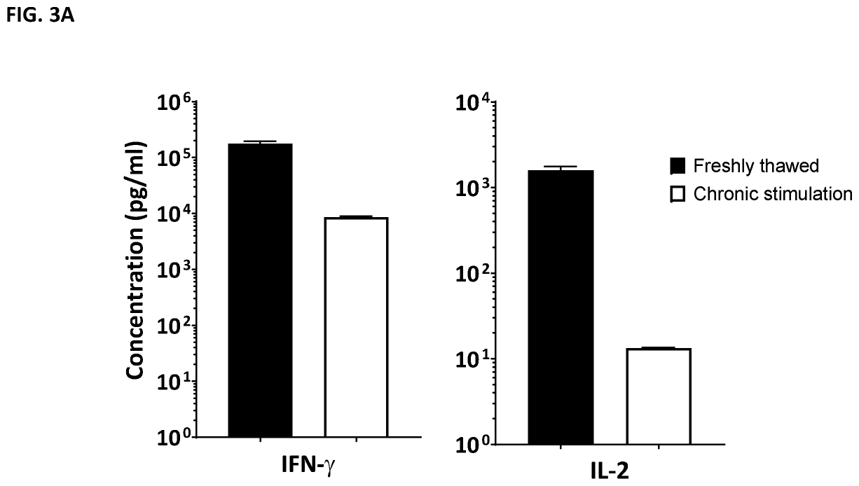

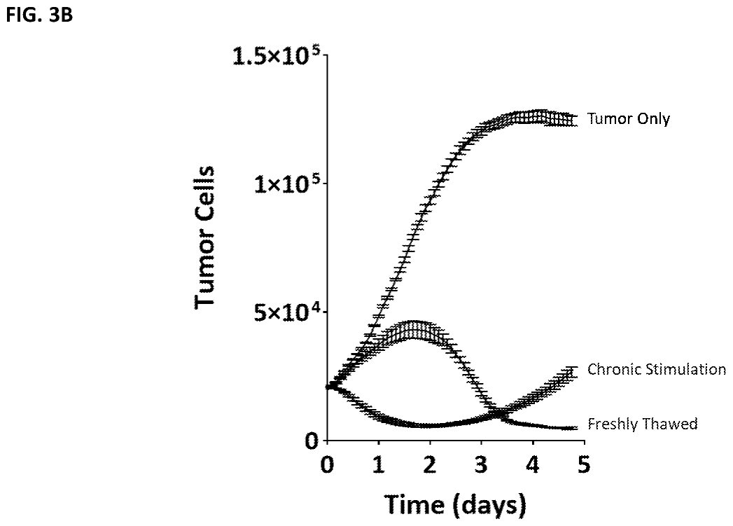

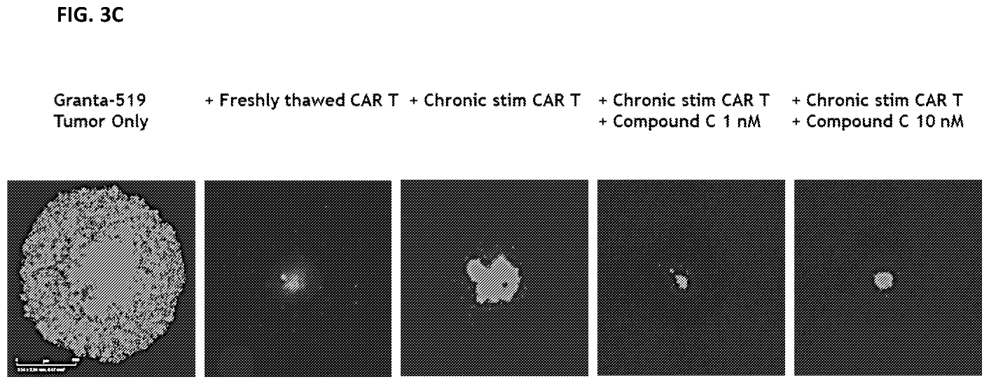

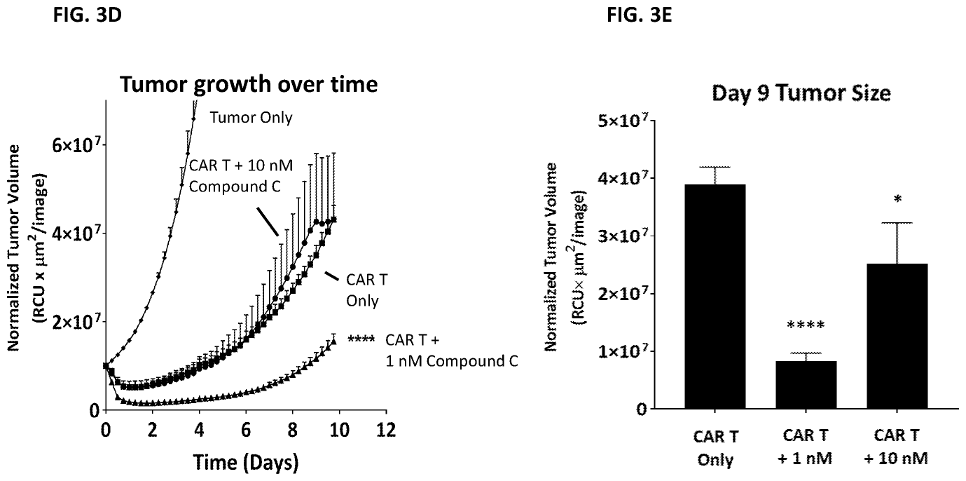

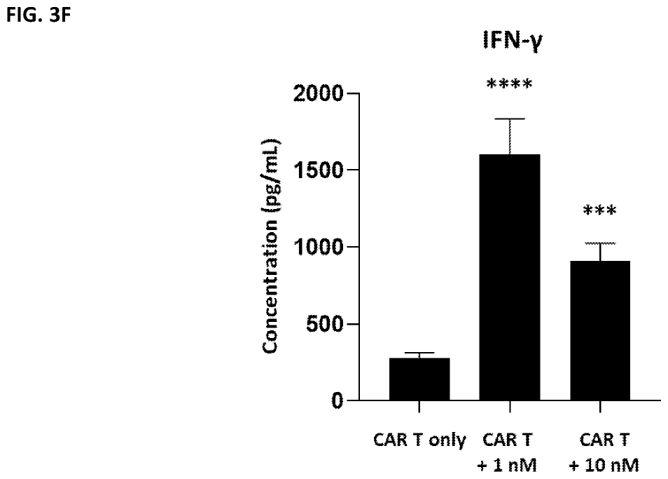

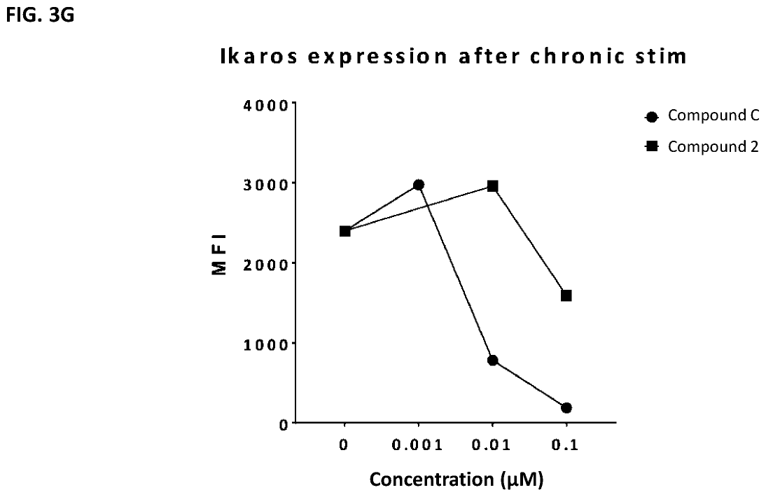

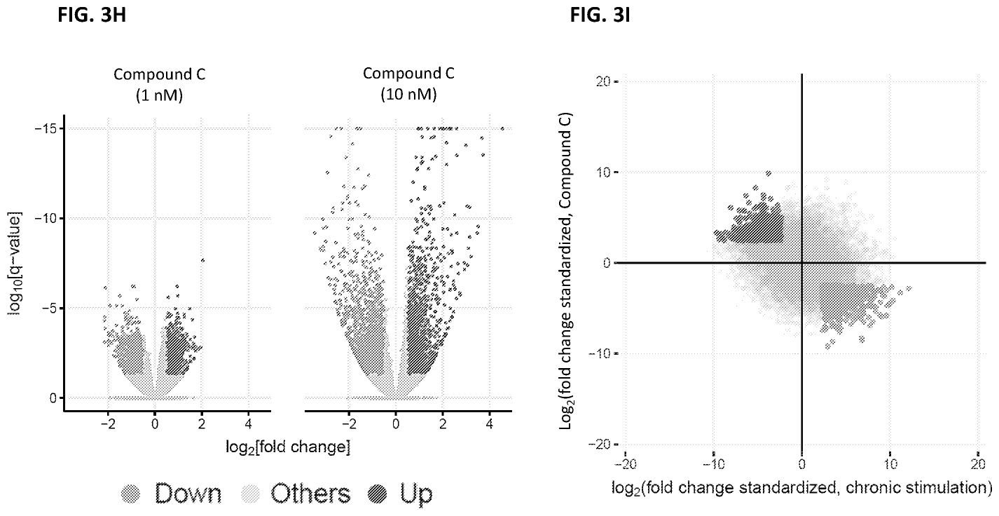

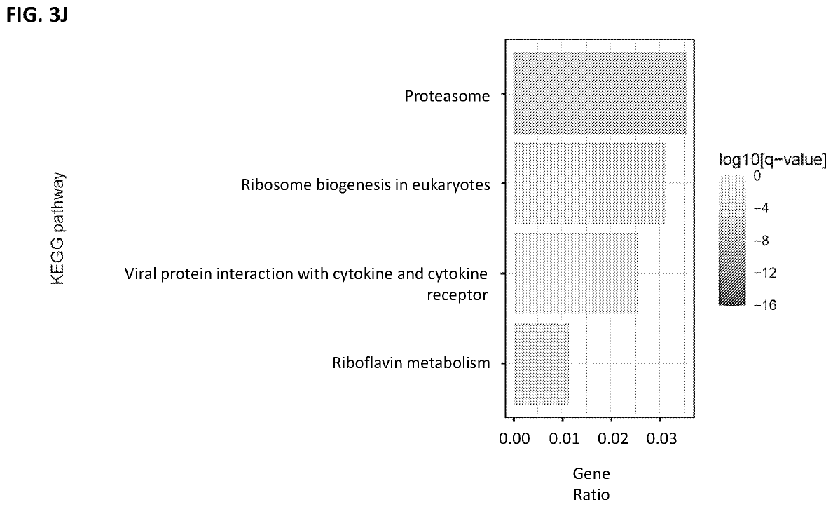

To simulate conditions of chronic stimulation, anti-CD19 CAR-expressing T cells, produced as described in Example 1, were incubated with plate-bound CAR-specific anti-idiotypic antibody (see, e.g., WO2018/023100) and incubated at 37° C. for a period of six days. This six-day stimulation has been shown to induce a hypofunctional state in stimulated CAR T cells. For instance, CAR T cells that were re-challenged with CD19+ tumor cells following the six-day stimulation had reduced IFNγ and IL-2 cytokine production (FIG. 3A) and reduced cytolytic function (FIG. 3B) compared to freshly thawed CAR T cells not subjected to the six-day stimulation.

The ability of Compound C to block or reduce this CAR T cell hypofunctionality following chronic stimulation was assessed. To do so, engineered CAR T cells were concurrently treated with Compound C, Compound 2, or a vehicle control during the six-day stimulation. Stimulated CAR T cells were then harvested, washed twice in media, and re-challenged with Granta-519 lymphoma tumor spheroids (that had been transduced with IncuCyte NucLight Red) in the absence of Compound C, after which cytolytic activity was assessed by loss of red fluorescence over time and averaged measurement of tumor spheroid size at various times following co-culture with CAR-T cells. Cytokine production, Ikaros expression, and gene expression of the CAR T Cells also were assessed.

FIG. 3C shows representative images of Granta-519 tumor spheroids at day 9 of co-culture with CAR T cells that were stimulated in the presence of Compound C. FIGS. 3D and 3E show the volume of tumor spheroids co-cultured with CAR T cells that were stimulated in the presence of Compound C after chronic stimulation. As shown in FIG. 3D and FIG. 3E, cytolytic activity during re-challenge was improved by treatment with Compound C at a 1 nM concentration during stimulation (**** P<0.0001). As shown in FIG. 3E, treatment with 10 nM of Compound C during stimulation also led to reduced tumor size at day 9 of co-culture (* P<0.05), though to a lesser extent than with 1 nM of Compound C (**** P<0.0001). Cytokine production of IFNγ in the supernatant of co-cultured cells was also assessed five days following re-challenge with tumor cells, and Compound C at a 1 nM concentration present concurrently during chronic stimulation also improved IFNγ production in the treated CAR T cells upon re-challenge (FIG. 3F, **** P<0.0001). Treatment with 10 nM of Compound C during stimulation also increased IFNγ production (*** P<0.001), though to a lesser extent than with 1 nM of Compound C.

FIG. 3G shows Ikaros expression in CAR T cells after six days of chronic stimulation in the presence of Compound C or Compound 2, as determined by MFI. As shown, Ikaros expression was lowest for CAR T cells treated with Compound C at higher concentrations (e.g., 0.01 μM and 0.1 μM).

Five days after re-challenge, CAR T cells were sorted from tumor spheroids, and gene expression was analyzed by RNA sequencing (RNA-seq). Libraries for RNA-seq were sequenced, and reads were mapped to the Genome Reference Consortium Human Build 38 (GRCh38) human reference genome. Gene expression levels were quantified, and differential expression was performed using DESeq2. Gene expression was compared between chronically stimulated CAR T cells treated with Compound C during stimulation and untreated chronically stimulated CAR T cells, as well as between untreated chronically stimulated CAR T cells and non-stimulated CAR T cells. Differential expression was calculated based on treatment effects with 1 nM and 10 nM of the compounds. Differential locus selection cut offs were q≤0.05 and absolute log₂ fold change ≥0.5.

As shown in FIG. 3H, treatment with Compound C during chronic stimulation led to altered gene expression in chronically stimulated CAR T cells, compared to untreated chronically stimulated CAR T cells. For the same set of genes, FIG. 3I shows how expression levels differed between treated and untreated chronically stimulated CAR T cells (y-axis) and between untreated chronically stimulated and non-stimulated CAR T cells (x-axis). As shown, genes upregulated by treatment with Compound C during stimulation were those downregulated by chronic stimulation (upper left quadrant), and genes downregulated by treatment with Compound C during stimulation were those upregulated by chronic stimulation (lower right quadrant).

Pathway analysis of CAR T RNA-seq data using the Kyoto Encyclopedia of Genes and Genomes (KEGG) was also performed using clusterProfiler. Pathways whose upregulation by chronic stimulation was prevented or reduced by treatment with Compound C are shown in FIG. 3J and include specific cell proliferation pathways. Gene ratios as shown in FIG. 3J were calculated as the percentage of significant genes over total genes in a given pathway.

Together, these results indicate that the presence of Compound C, even at relatively low concentrations, during conditions of chronic stimulation can limit or reduce exhaustion in chronically stimulated CAR T cells. These results also showed that Ikaros expression in CAR T cells was reduced by treatment with higher concentrations of Compound C, and that pathways involved in T cell function are enriched after chronic stimulation and can be reversed by Compound C with concurrent treatment during chronic stimulation.

These results also showed that treatment with a higher concentration (10 nM) of Compound C during chronic stimulation led to less improvement in CAR T cell cytolytic activity and cytokine production, compared to treatment with 1 nM of Compound C. Without wishing to be bound by theory, treatment with higher concentrations of Compound C may lead to sustained or excessive degradation of Ikaros and Aiolos, resulting in CAR T cell dysfunction that limits the benefits of Compound C treatment during chronic stimulation. In contrast, lower concentrations of Compound C may improve CAR T cell function without the detrimental effects of sustained or excessive Aiolos and Ikaros degradation.

### Example 4: Assessment of Chronically Stimulated CAR-T Cells Following Rescue Treatment with Compound C

The ability of Compound C to rescue chronically stimulated CAR T cells from exhaustion was assessed.

Anti-CD19 CAR-expressing T cells, produced as described in Example 1, were chronically stimulated as described in Example 3, but in the absence of Compound C during stimulation. Instead, chronically stimulated CAR T cells were treated with Compound C during re-challenge with Granta-519 lymphoma tumor spheroids or A549 tumor spheroids, after which cytolytic activity, cytokine production, and gene expression of the CAR T Cells was assessed.

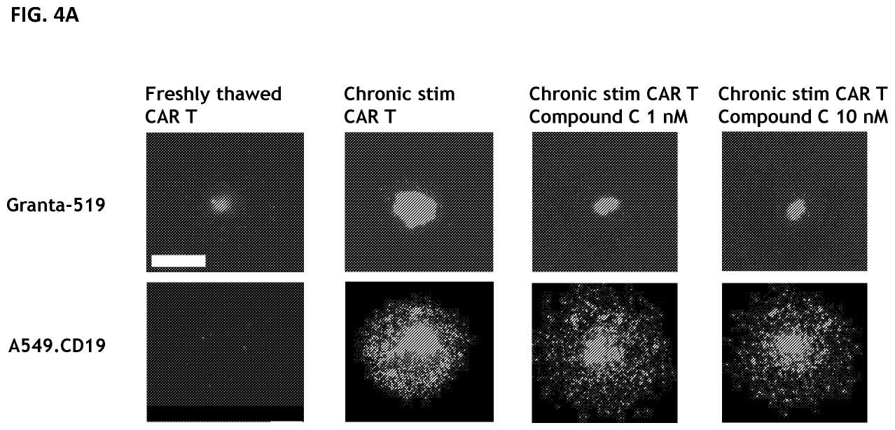

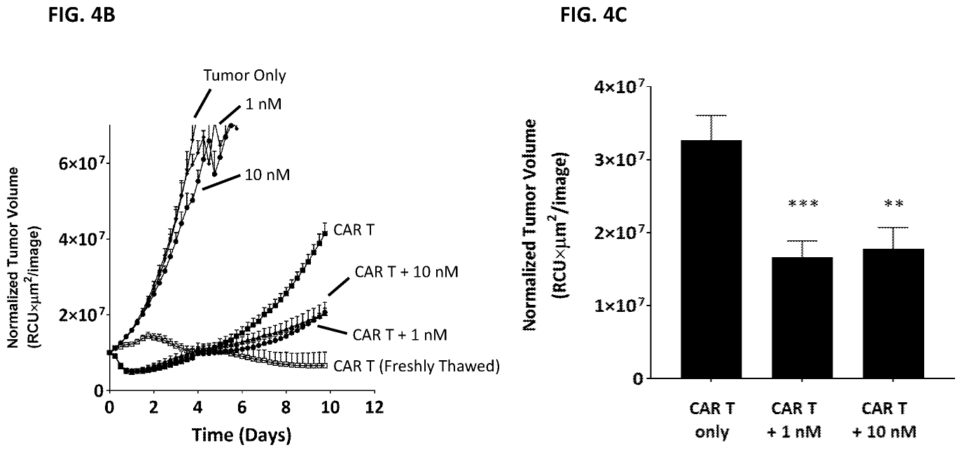

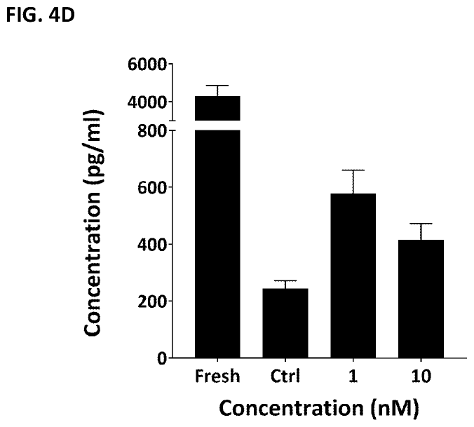

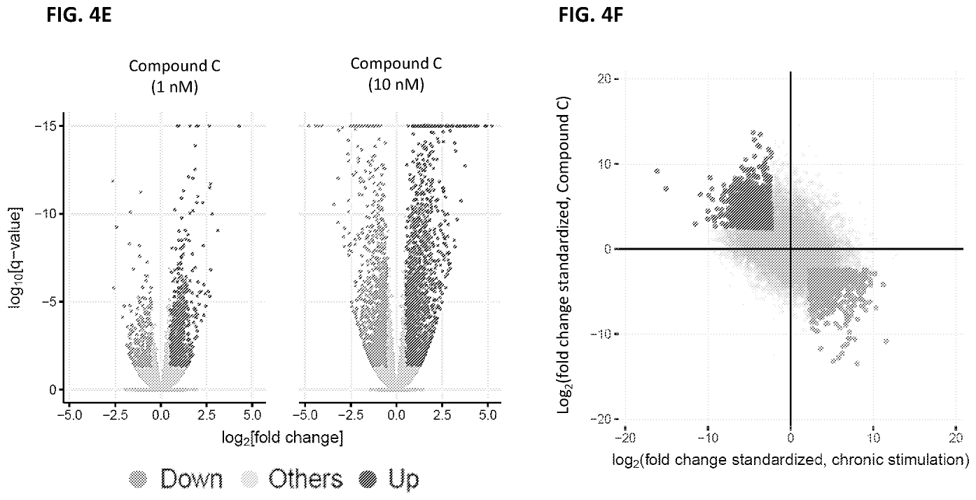

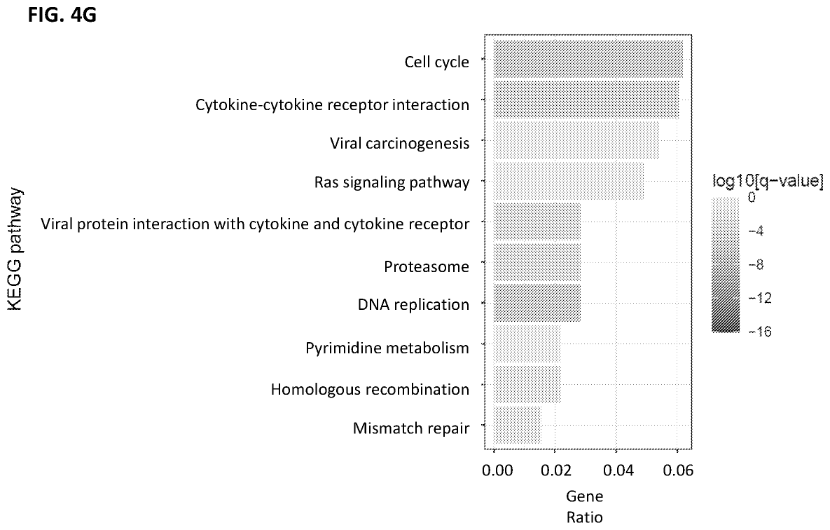

FIG. 4A shows representative images of Granta-519 and A549 tumor spheroids at day 9 of co-culture with CAR T cells in the presence of Compound C. FIGS. 4B and 4C show the volume of tumor spheroids co-cultured with anti-CD19 CAR T cells in the presence of Compound C. As shown in FIG. 4B, tumor volume was more reduced by simultaneous treatment with the CAR T cells and Compound C (1 nM or 10 nm) than it was by treatment with the chronically stimulated CAR T cells alone or Compound C alone (P<0.0001 for both concentrations). FIG. 4C shows similar results based on tumor size on day 9 of co-culture (* P<0.001 for 1 nM; ** P<0.01 for 10 nM). Cytokine production was also assessed five days following re-challenge with tumor cells, and the presence of Compound C during re-challenge also improved IFNγ production in the chronically stimulated CAR T cells, compared to chronically stimulated CAR T cells alone (control; FIG. 4D). Cytokine production was highest for CAR T cells re-challenged in the presence of Compound C at 1 nM (P<0.0001) and was also increased for CAR T cells re-challenged in the presence of Compound at 10 nM (P<0.05).

Gene expression of chronically stimulated CAR T cells was also analyzed by RNA sequencing as in Example 3. Gene expression was compared between chronically stimulated CAR T cells treated with Compound C during re-challenge and untreated chronically stimulated CAR T cells, as well as between untreated chronically stimulated CAR T cells and non-stimulated CAR T cells. As shown in FIG. 4E, treatment with Compound C during re-challenge led to altered gene expression in chronically stimulated CAR T cells, compared to untreated chronically stimulated CAR cells. As shown in FIG. 4F, genes upregulated by treatment with Compound C during re-challenge were those downregulated by chronic stimulation (upper left quadrant), and genes downregulated by treatment with Compound C during re-challenge were those upregulated by chronic stimulation (lower right quadrant). Pathways whose upregulation by chronic stimulation is reversed by treatment with Compound C during re-challenge are shown in FIG. 4G.

Together, these results indicate that the presence of Compound C, even at relatively low concentrations, can rescue or restore already chronically stimulated CAR T cells from an exhausted phenotype. Similar to results with concurrent stimulation described in Example 3, the results revealed that pathways involved in T cell function are enriched after chronic stimulation and can be reversed by Compound C with rescue treatment.

### Example 5: Assessment of Chronically Stimulated CAR-T Cells Treated with Compound C at Different Concentration Levels

Different concentrations of Compound C were tested for their effects on cytolytic function of CAR T cells following chronic stimulation. CAR T cells were treated with various concentrations of Compound C either during or after chronic stimulation.

Anti-CD19 CAR-expressing T cells, produced as described in Example 1, were chronically stimulated as described in Example 3 (with Compound C present during chronic stimulation), then re-challenged with Granta-519 lymphoma tumor spheroids in the absence of Compound C (concurrent treatment group). In addition, anti-CD19 CAR-expressing T cells were chronically stimulated as described in Example 4 (with Compound C absent during chronic stimulation), then re-challenged with Granta-519 lymphoma tumor spheroids in the presence of Compound C (rescue treatment group). During re-challenge, cytolytic activity was assessed by tumor cell number at various times during co-culture with CAR-T cells.

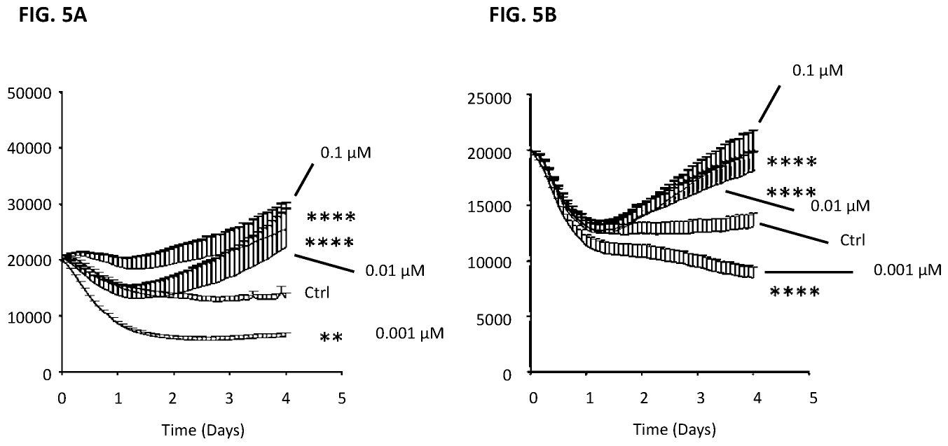

FIG. 5A shows tumor cell number during re-challenge of CAR T cells chronically stimulated in the presence of Compound C (concurrent treatment). FIG. 5B shows tumor cell number during re-challenge of CAR T cells in the presence of Compound C (rescue treatment). In both treatment conditions, tumor cell number was reduced compared to control conditions for CAR T cells treated with Compound C at a 0.001 μM concentration. Also in both treatment groups, tumor cell number was higher compared to control conditions for CAR T cells treated with Compound C at 0.01 μM and 0.1 μM concentrations.

These results suggest that treatment with higher concentrations of Compound C may impair cytolytic activity of chronically stimulated CAR T cells. Without wishing to be bound by theory, this impairment may be due sustained or excessive degradation of Ikaros by Compound C at higher concentrations, for instance as shown in Example 3.

### Example 6: Assessment of an Intermittent Dosing Schedule for Compound C

Due to the robust impact of Compound C on degradation of Ikaros and Aiolos, in which excessive or sustained treatment may impact CAR T cell function negatively and/or cause neutropenia secondary to neutrophil maturation arrest, an intermittent dosing schedule for treatment with Compound C was evaluated for its ability to effect deep, but transient degradation of Ikaros. To do so, the pharmacokinetics of a weekly (Q7D) 0.3-mg dose of Compound C were modelled. Based on these results, Ikaros degradation in CAR T cells after short (24 hours) and chronic (six days) exposure to Compound C was determined. The effects of a weekly 0.3-mg dose of Compound C on neutrophil development were also modelled.

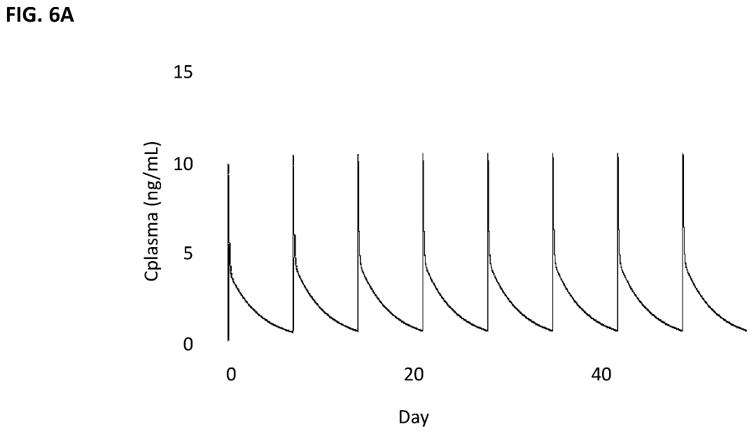

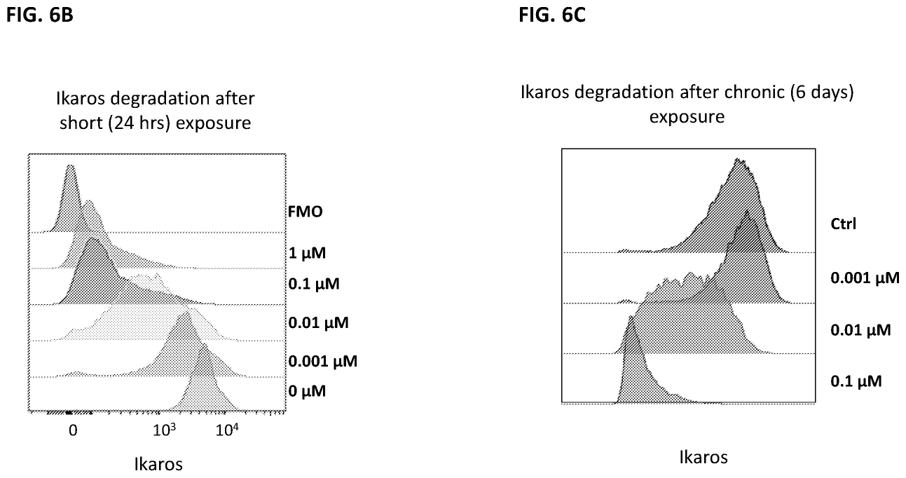

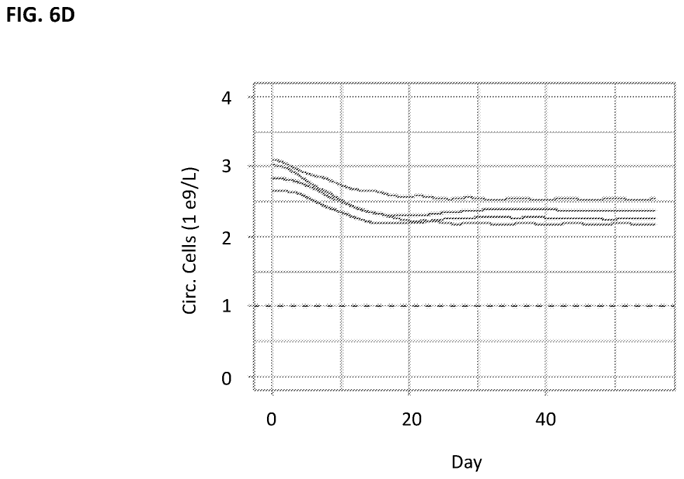

FIG. 6A shows expected plasma concentration levels of Compound C based on a Q7D dosing regimen. As shown, pharmacokinetic modelling revealed biphasic elimination of Compound C, with a high transient initial peak to a Cₘₐₓ of 20 nM, followed by extended terminal elimination and a Cₘᵢₙ at Day 7 of approximately 1 nM.

FIGS. 6B and 6C show Ikaros levels in anti-CD19 CAR T cells, generated as described in Example 1, following 24-hour (FIG. 6B) or six-day exposure (FIG. 6C) to Compound C. As shown in FIG. 6B, dose levels as low as 0.01 μM of Compound C lead to Ikaros degradation, suggesting that a 0.3-mg dose of Compound C (with a Cₘₐₓ of 20 nM) would lead to substantial Ikaros degradation after 24 hours. As shown in FIG. 6C, a six-day exposure to 0.001 μM of Compound C did not affect Ikaros levels, suggesting that as plasma concentrations of Compound C decrease over time between doses, Ikaros levels may have the opportunity to return to baseline levels after initial degradation.

FIG. 6D shows predicted absolute neutrophil count (ANC) profiles generated using four different pharmacodynamic models. These profiles were generated in order to assess the effects of a weekly 0.3-mg dose of Compound C on neutrophil development, including to assess whether the weekly 0.3-mg dose would result in low neutrophil count (e.g., neutropenia). For all models, the mean nadir ANC level was predicted to be above approximately 2.2×10⁹ cells/L, given a weekly 0.3-mg dose.

Together, these results suggest that an intermittent dosing schedule for Compound C in combination with a CAR T cell therapy would allow for deep, but transient Ikaros degradation in CAR T cells without substantially affecting neutrophil development or negatively affecting CAR T cell function. Without wishing to be bound by theory, pharmacokinetic elimination of Compound C with a weekly dose may allow for Ikaros levels to return to baseline values between doses, thereby preventing CAR T cell dysfunction or neutrophil maturation arrest due to sustained Ikaros degradation.

### Example 7: Assessment of an Intermittent Dosing Schedule for Compound C in Chronically Stimulated CAR T Cells

The effects of an intermittent Compound C dosing schedule on CAR T cells was assessed. To simulate the biphasic elimination of Compound C with weekly doses as shown in Example 6, CAR T cells were chronically stimulated in the presence of Compound C for one day at a high concentration of Compound C, then for five days at a low concentration of Compound C.

Anti-CD19 CAR-expressing T cells, produced as described in Example 1, were chronically stimulated as described in Example 3 in the presence of Compound C. During chronic stimulation, Compound C was present at (i) 50 nM for one day and 1 nM for the remaining five days, (ii) 25 nM for one day and 0.5 nM for the remaining five days, (iii) 10 nM for one day and absent for the remaining five days, or (iv) 1 nM for all six days. Stimulated CAR T cells were then re-challenged with Raji lymphoma tumor spheroids in the absence of Compound C, after which cytolytic activity was assessed.

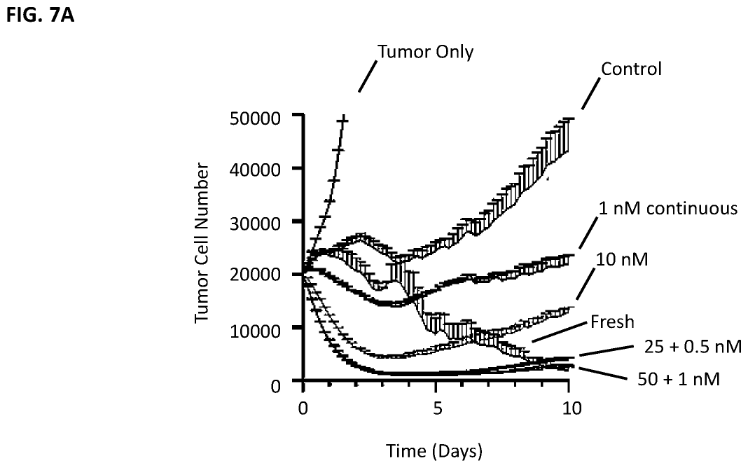

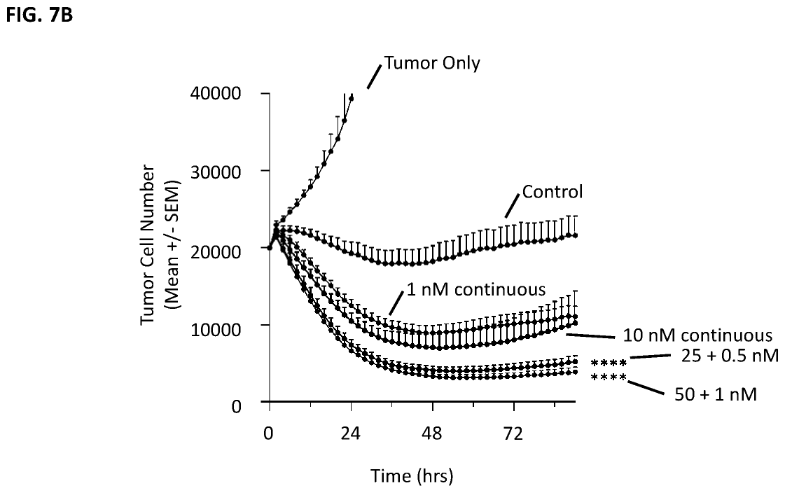

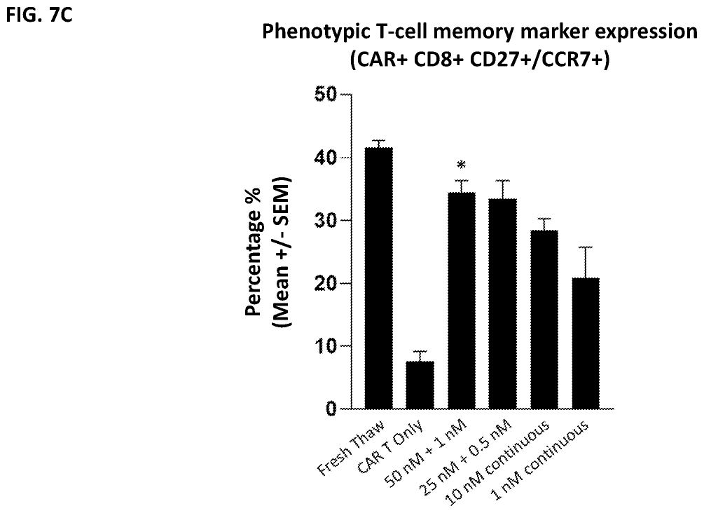

FIG. 7A shows tumor cell number during re-challenge of CAR T cells chronically stimulated in the presence of Compound C as described, as well as fresh (non-chronically stimulated) CAR T cells. As shown, the presence of Compound C at a high concentration for one day followed by a low concentration for five days during chronic stimulation (50 nM for one day and 1 nM for the remaining five days, or 25 nM for one day and 0.5 nM for the remaining five days) led to the most increase in cytolytic activity of chronically stimulated CAR T cells.

Similar results are shown in FIG. 7B for a separate experiment in which CAR T cells were chronically stimulated for 7 days in the presence or absence of Compound C. In this experiment, Compound C was present at (i) 50 nM for one day and 1 nM for the remaining six days of stimulation, (ii) 25 nM for one day and 0.5 nM for the remaining six days of stimulation, (iii) 10 nM for all seven days of stimulation, or (iv) 1 nM for all seven days of stimulation. Stimulated CAR T cells were then re-challenged with Raji lymphoma tumor spheroids in the absence of Compound C, after which cytolytic activity was assessed over a four-day period. As shown in FIG. 7B, the presence of Compound C at 50 nM for one day and 1 nM for the remaining six days of stimulation improved cytolytic activity of the chronically stimulated CAR T cells (**** P<0.0001). The presence of Compound C at 25 nM for one day and 0.5 nM for the remaining six days of stimulation also improved cytolytic activity (**** P<0.0001). The CAR T cells stimulated for seven days in the presence or absence of Compound C were also analyzed for phenotypic T cell memory markers using flow cytometry following chronic stimulation. As shown in FIG. 7C, CAR-expressing CD8 T cells stimulated in the presence of Compound C had higher percentages of CD27+/CCR7+ cells than did cells stimulated in the absence of Compound C. CD27/CCR7 expression, which is a marker for less differentiated, naïve-like T cells, was highest in CAR-expressing CD8 T cells stimulated in the presence of Compound C at 50 nM for one day and 1 nM for the remaining six days of stimulation (* P<0.05).

These results suggest that weekly doses of Compound C, for instance as part of an intermittent dosing regimen for Compound C, would improve cytolytic activity of CAR T cells administered in combination with Compound C. These results also indicate that an intermittent dosing regimen for Compound C would lead to the preservation of a less differentiated memory phenotype in CAR T cells.

### Example 8: Treatment of Relapsed/Refractory B-Cell Malignancies with Anti-CD19 CAR T Cells and Compound C

A combination therapy that includes Compound C and therapeutic CAR T cell compositions containing autologous T cells expressing a CAR specific for CD19 was administered to subjects with relapsed/refractory aggressive B-cell Non-Hodgkin's Lymphoma (NHL).

Specifically, autologous anti-CD19 directed therapeutic T cell compositions were generated as described in Example 1 from leukapheresis samples from adult human subjects with aggressive B-cell NHL, defined as DLBCL NOS, including transformed indolent NHL; follicular lymphoma Grade 3B; T cell/histiocyte-rich large B-cell lymphoma; EBV positive DLBCL, NOS; primary mediastinal (thymic) large B-cell lymphoma; or high grade B-cell lymphoma with MYC and BCL2 and/or BCL6 rearrangements with DLBCL histology (double/triple-hit lymphoma). Aggressive B-cell NHL was histologically confirmed at last relapse, otherwise a new tumor biopsy was required.

Eligible subjects had relapsed or been refractory to at least two prior lines of systemic therapy that included a CD20-targeted agent and an anthracycline. For subjects with transformed disease, the subject should have had at least two lines of systemic therapy for transformed disease (i.e., the DLBCL) to be eligible. Prior lines of therapy did not include those given for a previously indolent condition (e.g., follicular lymphoma or CLL). Subjects did not have to have received anthracycline for their DLBCL if anthracycline was received for indolent disease. Subjects did not have prior treatment with Compound C.

Eligible subjects had positron emission tomography (PET)-positive (i.e., Deauville score 4 or 5) and computed tomography (CT) measurable disease as per Lugano Classification. Eligible subjects had a sum of product of perpendicular diameters (SPD) of up to six index lesions that was greater than or equal to 25 cm² by CT scan.

For the production of the autologous anti-CD19 CAR T cell compositions, leukapheresis samples were obtained from subjects approximately four weeks prior to scheduled CAR T cell administration. Subjects then received a lymphodepleting chemotherapy with fludarabine (Flu, 30 mg/m²) and cyclophosphamide (Cy, 300 mg/m²) for three days. Two to seven days after completion of lymphodepleting chemotherapy, the subjects received the dose of CAR-expressing T cells (e.g., 1×10⁶ total T cells) on Day 1 of the combination therapy.

Subjects were assigned to one of seven dosing subcohorts for Compound C administration. Patients assigned to Subcohort A received a dosing regimen of 0.3 mg of Compound C once weekly beginning on Day 8. For subjects with Grade 3 or greater CRS or NT at the scheduled start of Compound C administration, the initiation of Compound C administration can be delayed by up to three days. Depending on the safety and tolerability of the dosing regimen for Subcohort A, other dosing regimens can be evaluated. Subcohort B receives a dosing regimen of 0.3 mg of Compound C once weekly beginning on Day 1. Subcohort C receives a dosing regimen of 0.3 mg of Compound C once weekly beginning on Day 15. Subcohort D receives a dosing regimen of 0.4 mg of Compound C once weekly beginning on Day 8. Subcohort E receives a dosing regimen of 0.3 mg of Compound C once every two weeks beginning on Day 8. Subcohort F receives a dosing regimen of 0.2 mg of Compound C once weekly beginning on Day 8. Subcohort G receives a dosing regimen of 0.6 mg of Compound C once weekly beginning on Day 8. For all subcohorts, Compound C administration is continued until Day 85 of the combination therapy.

Subjects were monitored for safety, pharmacokinetics and pharmacodynamics, and clinical response. Determining clinical response to the treatment included assessing whether the subject exhibited progressive disease (PD), partial response (PR), complete response (CR), and/or stable disease.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | ESKYGPPCPPCP | spacer |
| | | (IgG4hinge) (aa) |
| 2 | gaatctaagtacggaccgccctgccccccttgccct | spacer |
| | | (IgG4hinge) (nt) |
| 3 | ESKYGPPCPPCPGQPREPQVYTLPPSQEEMTKNQVSLTCLV | Hinge-CH3 |
| | KGFYPSDIAVEWESNGQPENNYKTTPPVLDSDGSFFLYSR | spacer |
| | LTVDKSRWQEGNVFSCSVMHEALHNHYTQKSLSLSLGK | |
| 4 | ESKYGPPCPPCPAPEFLGGPSVFLFPPKPKDTLMISRTPEVT | Hinge-CH2-CH3 |
| | CVVVDVSQEDPEVQFNWYVDGVEVHNAKTKPREEQFNS | spacer |
| | TYRVVSVLTVLHQDWLNGKEYKCKVSNKGLPSSIEKTISK | |
| | AKGQPREPQVYTLPPSQEEMTKNQVSLTCLVKGFYPSDIA | |
| | VEWESNGQPENNYKTTPPVLDSDGSFFLYSRLTVDKSRW | |
| | QEGNVFSCSVMHEALHNHYTQKSLSLSLGK | |
| 5 | RWPESPKAQASSVPTAQPQAEGSLAKATTAPATTRNTGRG | IgD-hinge-Fc |
| | GEEKKKEKEKEEQEERETKTPECPSHTQPLGVYLLTPAVQ | |
| | DLWLRDKATFTCFVVGSDLKDAHLTWEVAGKVPTGGVE | |
| | EGLLERHSNGSQSQHSRLTLPRSLWNAGTSVTCTLNHPSL | |
| | PPQRLMALREPAAQAPVKLSLNLLASSDPPEAASWLLCEV | |
| | SGFSPPNILLMWLEDQREVNTSGFAPARPPPQPGSTTFWA | |
| | WSVLRVPAPPSPQPATYTCVVSHEDSRTLLNASRSLEVSY | |
| | VTDH | |
| 6 | LEGGGEGRGSLLTCGDVEENPGPR | T2A |
| 7 | MLLLVTSLLLCELPHPAFLLIPRKVCNGIGIGEFKDSLSINA | tEGFR |
| | TNIKHFKNCTSISGDLHILPVAFRGDSFTHTPPLDPQELDIL | |
| | KTVKEITGFLLIQAWPENRTDLHAFENLEIIRGRTKQHGQF | |
| | SLAVVSLNITSLGLRSLKEISDGDVIISGNKNLCYANTINW | |
| | KKLFGTSGQKTKIISNRGENSCKATGQVCHALCSPEGCWG | |
| | PEPRDCVSCRNVSRGRECVDKCNLLEGEPREFVENSECIQC | |
| | HPECLPQAMNITCTGRGPDNCIQCAHYIDGPHCVKTCPAG | |
| | VMGENNTLVWKYADAGHVCHLCHPNCTYGCTGPGLEGC | |
| | PTNGPKIPSIATGMVGALLLLLVVALGIGLFM | |
| 8 | FWVLVVVGGVLACYSLLVTVAFIIFWV | CD28 (amino |
| | | acids 153-179 of |
| | | Accession No. |
| | | P10747) |
| 9 | IEVMYPPPYLDNEKSNGTIIHVKGKHLCPSPLFPGPSKP | CD28 (amino |
| | FWVLVVVGGVLACYSLLVTVAFIIFWV | acids 114-179 of |
| | | Accession No. |
| | | P10747) |
| 10 | RSKRSRLLHSDYMNMTPRRPGPTRKHYQPYAPPRDFAAY | CD28 (amino |
| | RS | acids 180-220 of |
| | | P10747) |
| 11 | RSKRSRGGHSDYMNMTPRRPGPTRKHYQPYAPPRDFAAY | CD28 (LL to |
| | RS | GG) |
| 12 | KRGRKKLLYIFKQPFMRPVQTTQEEDGCSCRFPEEEEGGC | 4-1BB (amino |
| | EL | acids 214-255 of |
| | | Q07011.1) |
| 13 | RVKFSRSADAPAYQQGQNQLYNELNLGRREEYDVLDKRR | CD3 zeta |
| | GRDPEMGGKPRRKNPQEGLYNELQKDKMAEAYSEIGMK | |
| | GERRRGKGHDGLYQGLSTATKDTYDALHMQALPPR | |
| 14 | RVKFSRSAEPPAYQQGQNQLYNELNLGRREEYDVLDKRR | CD3 zeta |
| | GRDPEMGGKPRRKNPQEGLYNELQKDKMAEAYSEIGMK | |
| | GERRRGKGHDGLYQGLSTATKDTYDALHMQALPPR | |
| 15 | RVKFSRSADAPAYKQGQNQLYNELNLGRREEYDVLDKRR | CD3 zeta |
| | GRDPEMGGKPRRKNPQEGLYNELQKDKMAEAYSEIGMK | |
| | GERRRGKGHDGLYQGLSTATKDTYDALHMQALPPR | |
| 16 | RKVCNGIGIGEFKDSLSINATNIKHFKNCTSISGDLHILPVA | tEGFR |
| | FRGDSFTHTPPLDPQELDILKTVKEITGFLLIQAWPENRTDL | |
| | HAFENLEIIRGRTKQHGQFSLAVVSLNITSLGLRSLKEISDG | |
| | DVIISGNKNLCYANTINWKKLFGTSGQKTKIISNRGENSCK | |
| | ATGQVCHALCSPEGCWGPEPRDCVSCRNVSRGRECVDKC | |
| | NLLEGEPREFVENSECIQCHPECLPQAMNITCTGRGPDNCI | |
| | QCAHYIDGPHCVKTCPAGVMGENNTLVWKYADAGHVCH | |
| | LCHPNCTYGCTGPGLEGCPTNGPKIPSIATGMVGALLLLLV | |
| | VALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | -PGGG-(SGGGG)₅-P- wherein P is proline, G is glycine and S is | Linker |
| | serine | |
| 23 | GSADDAKKDAAKKDGKS | Linker |
| 24 | atgcttctcctggtgacaagccttctgctctgtgagttaccacacccagcat | GMCSFR alpha |
| | tcctcctgatccca | chain signal |
| | | sequence |
| 25 | MLLLVTSLLLCELPHPAFLLIP | GMCSFR alpha |
| | | chain signal |
| | | sequence |
| 26 | MALPVTALLLPLALLLHA | CD8 alpha signal |
| | | peptide |
| 27 | Glu Pro Lys Ser Cys Asp Lys Thr His Thr Cys Pro Pro Cys Pro | Hinge |
| 28 | Glu Arg Lys Cys Cys Val Glu Cys Pro Pro Cys Pro | Hinge |
| 29 | ELKTPLGDTHTCPRCPEPKSCDTPPPCPRCPEPKSCDTPPPC | Hinge |
| | PRCPEPKSCDTPPPCPRCP | |
| 30 | Glu Ser Lys Tyr Gly Pro Pro Cys Pro Ser Cys Pro | Hinge |
| 31 | Glu Ser Lys Tyr Gly Pro Pro Cys Pro Pro Cys Pro | Hinge |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro Cys Pro | Hinge |
| 35 | RASQDISKYLN | CDR L1 |
| 36 | SRLHSGV | CDR L2 |
| 37 | GNTLPYTFG | CDR L3 |
| 38 | DYGVS | CDR H1 |
| 39 | VIWGSETTYYNSALKS | CDR H2 |
| 40 | YAMDYWG | CDR H3 |
| 41 | EVKLQESGPGLVAPSQSLSVTCTVSGVSLPDYGVSWIRQPP | VH |
| | RKGLEWLGVIWGSETTYYNSALKSRLTIIKDNSKSQVFLK | |
| | MNSLQTDDTAIYYCAKHYYYGGSYAMDYWGQGTSVTVS | |
| | S | |
| 42 | DIQMTQTTSSLSASLGDRVTISCRASQDISKYLNWYQQKP | VL |
| | DGTVKLLIYHTSRLHSGVPSRFSGSGSGTDYSLTISNLEQE | |
| | DIATYFCQQGNTLPYTFGGGTKLEIT | |
| 43 | DIQMTQTTSSLSASLGDRVTISCRASQDISKYLNWYQQKP | scFv |
| | DGTVKLLIYHTSRLHSGVPSRFSGSGSGTDYSLTISNLEQE | |
| | DIATYFCQQGNTLPYTFGGGTKLEITGSTSGSGKPGSGEGS | |
| | TKGEVKLQESGPGLVAPSQSLSVTCTVSGVSLPDYGVSWI | |
| | RQPPRKGLEWLGVIWGSETTYYNSALKSRLTIIKDNSKSQ | |
| | VFLKMNSLQTDDTAIYYCAKHYYYGGSYAMDYWGQGTS | |
| | VTVSS | |
| 44 | KASQNVGTNVA | CDR L1 |
| 45 | SATYRNS | CDR L2 |
| 46 | QQYNRYPYT | CDR L3 |
| 47 | SYWMN | CDR H1 |
| 48 | QIYPGDGDTNYNGKFKG | CDR H2 |
| 49 | KTISSVVDFYFDY | CDR H3 |
| 50 | EVKLQQSGAELVRPGSSVKISCKASGYAFSSYWMNWVKQ | VH |
| | RPGQGLEWIGQIYPGDGDTNYNGKFKGQATLTADKSSST | |
| | AYMQLSGLTSEDSAVYFCARKTISSVVDFYFDYWGQGTT | |
| | VTVSS | |
| 51 | DIELTQSPKFMSTSVGDRVSVTCKASQNVGTNVAWYQQK | VL |
| | PGQSPKPLIYSATYRNSGVPDRFTGSGSGTDFTLTITNVQS | |
| | KDLADYFCQQYNRYPYTSGGGTKLEIKR | |
| 52 | GGGGSGGGGSGGGGS | Linker |
| 53 | EVKLQQSGAELVRPGSSVKISCKASGYAFSSYWMNWVKQ | scFv |
| | RPGQGLEWIGQIYPGDGDTNYNGKFKGQATLTADKSSST | |
| | AYMQLSGLTSEDSAVYFCARKTISSVVDFYFDYWGQGTT | |
| | VTVSSGGGGSGGGGSGGGGSDIELTQSPKFMSTSVGDRVS | |
| | VTCKASQNVGTNVAWYQQKPGQSPKPLIYSATYRNSGVP | |
| | DRFTGSGSGTDFTLTITNVQSKDLADYFCQQYNRYPYTSG | |
| | GGTKLEIKR | |
| 54 | HYYYGGSYAMDY | HC-CDR3 |
| 55 | HTSRLHS | LC-CDR2 |
| 56 | QQGNTLPYT | LC-CDR3 |
| 57 | gacatccagatgacccagaccacctccagcctgagcgccagcctgggcgaccgggtgac | Sequence |
| | catcagctgccgggccagccaggacatcagcaagtacctgaactggtatcagcagaagcc | encoding scFv |
| | cgacggcaccgtcaagctgctgatctaccacaccagccggctgcacagcggcgtgcccag | |
| | ccggtttagcggcagcggctccggcaccgactacagcctgaccatctccaacctggaaca | |
| | ggaagatatcgccacctacttttgccagcagggcaacacactgccctacacctttggcggcg | |
| | gaacaaagctggaaatcaccggcagcacctccggcagcggcaagcctggcagcggcga | |
| | gggcagcaccaagggcgaggtgaagctgcaggaaagcggccctggcctggtggccccc | |
| | agccagagcctgagcgtgacctgcaccgtgagcggcgtgagcctgcccgactacggcgt | |
| | gagctggatccggcagccccccaggaagggcctggaatggctgggcgtgatctggggca | |
| | gcgagaccacctactacaacagegccctgaagagccggctgaccatcatcaaggacaaca | |
| | gcaagagccaggtgttcctgaagatgaacagcctgcagaccgacgacaccgccatctacta | |
| | ctgcgccaagcactactactacggcggcagctacgccatggactactggggccagggcac | |
| | cagcgtgaccgtgagcagc | |
| 58 | X₁PPX₂P | Hinge |
| | X₁ is glycine, cysteine or arginine | |
| | X₂ is cysteine or threonine | |
| 59 | GSTSGSGKPGSGEGSTKG | Linker |

1. A method of treating a cancer expressing CD19, the method comprising administering to a subject having a cancer expressing CD19 a combination therapy comprising:
(i) a T cell therapy comprising a dose of engineered cells comprising T cells expressing a chimeric antigen receptor (CAR) directed against the cancer, wherein the T cell therapy is administered on Day 1 of the combination therapy; and
(ii) a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof.

2. A method of treating a lymphoma, the method comprising administering to a subject having a cancer that is a lymphoma a combination therapy comprising:
(i) a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
(ii) a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof.

3. The method of claim 1 or claim 2, wherein the administration of the compound is initiated subsequently to the administration of the T cell therapy.

4. A method of treating a cancer expressing CD19, the method comprising administering to a subject having a cancer expressing CD19 a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) directed against the cancer, wherein the T cell therapy is administered on Day 1 of the combination therapy, and the administration of the compound is initiated subsequently to the administration of the T cell therapy.

5. A method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy, and the administration of the compound is initiated subsequently to the administration of the T cell therapy.

6. The method of any one of claims 1-5, wherein the administration of the compound is initiated between Day 1 and Day 29, inclusive, of the combination therapy.

7. The method of any one of claims 1-6, wherein the compound is administered as a plurality of doses, wherein each dose is between at or about 0.1 mg and at or about 0.6 mg, inclusive.

8. The method of any one of claims 1-7, wherein the compound is administered as a plurality of intermittent doses administered no more than once weekly.

9. A method of treating a lymphoma, the method comprising administering to a subject having a cancer that is a lymphoma a combination therapy comprising:
(i) a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
(ii) a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein the compound is administered as a plurality of intermittent doses administered no more than once weekly, wherein each dose is between at or about 0.1 mg and at or about 0.6 mg, inclusive, wherein the administration of the compound is initiated between Day 1 and Day 29, inclusive, of the combination therapy.

10. A method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered no more than once weekly, wherein each dose is between at or about 0.1 mg and at or about 0.6 mg, inclusive, and wherein the administration of the compound is initiated between Day 1 and Day 29, inclusive, of the combination therapy.

11. The method of any one of claims 1-10, wherein the administration of the compound is initiated between Day 1 and Day 22, inclusive.

12. The method of any one of claims 1-11, wherein the administration of the compound is initiated between Day 1 and Day 15, inclusive.

13. The method of any one of claims 1-12, wherein the administration of the compound is initiated between Day 8 and Day 15, inclusive.

14. The method of any one of claims 1-12, wherein the administration of the compound is initiated at or about Day 1.

15. The method of any one of claims 1-13, wherein the administration of the compound is initiated at or about Day 8.

16. The method of any one of claims 1-13, wherein the administration of the compound is initiated at or about Day 15.

17. The method of any one of claims 1-16, wherein each dose of the plurality of intermittent doses is the same.

18. The method of any one of claims 1-17, wherein the compound is administered once weekly.

19. The method of any one of claims 1-18, wherein the compound is administered once every 7 days (Q7D).

20. The method of any one of claims 1-17, wherein the compound is administered once every two weeks.

21. The method of any one of claims 1-17 and 20, wherein the compound is administered once every 14 days (Q14D).

22. The method of any one of claims 1-21, wherein the compound is administered for at least 12 weeks after the administration of the T cell therapy.

23. The method of any one of claims 1-21, wherein the compound is administered for up to 12 weeks after the administration of the T cell therapy.

24. The method of any one of claims 1-12, 14, 17-19, 22, and 23, wherein the compound is administered on Days 1, 8, 15, 22, 29, 36, 43, 50, 57, 64, 71, 78, and 85.

25. The method of any one of claims 1-13, 15, 17-19, 22, and 23, wherein the compound is administered on Days 8, 15, 22, 29, 36, 43, 50, 57, 64, 71, 78, and 85.

26. The method of any one of claims 1-13, 16-19, 22, and 23, wherein the compound is administered on Days 15, 22, 29, 36, 43, 50, 57, 64, 71, 78, and 85.

27. The method of any one of claims 1-13, 15, 17, and 20-23, wherein the compound is administered on Days 8, 22, 36, 50, 64, and 78.

28. The method of any one of claims 1-27, wherein the dose of the compound is between at or about 0.3 mg and at or about 0.6 mg, inclusive.

29. The method of any one of claims 1-28, wherein the dose of the compound is at or about 0.6 mg.

30. The method of any one of claims 1-27, wherein the dose of the compound is between at or about 0.2 mg and at or about 0.4 mg, inclusive.

31. The method of any one of claims 1-28 and 30, wherein the dose of the compound is at or about 0.4 mg.

32. The method of any one of claims 1-28 and 30, wherein the dose of the compound is less than 0.4 mg.

33. The method of any one of claims 1-28, 30, and 32, wherein the dose of the compound is at or about 0.3 mg.

34. The method of any one of claims 1-28, 30, and 32, wherein the dose of the compound is at or about 0.2 mg.

35. A method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 8, 15, 22, 29, 36, 43, 50, 57, 64, 71, 78, and 85, wherein each dose is 0.3 mg.

36. A method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 1, 8, 15, 22, 29, 36, 43, 50, 57, 64, 71, 78, and 85, wherein each dose is 0.3 mg.

37. A method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 15, 22, 29, 36, 43, 50, 57, 64, 71, 78, and 85, wherein each dose is 0.3 mg.

38. A method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 8, 15, 22, 29, 36, 43, 50, 57, 64, 71, 78, and 85, wherein each dose is 0.4 mg.

39. A method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every 14 days (Q14D) and is administered on Days 8, 22, 36, 50, 64, and 78, wherein each dose is 0.3 mg.

40. A method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 8, 15, 22, 29, 36, 43, 50, 57, 64, 71, 78, and 85, wherein each dose is 0.2 mg.

41. A method of treating a lymphoma, the method comprising administering to a subject having a cancer that is a lymphoma a compound that is (S)-2-(2,6-dioxopiperidin-3-yl)-4-((2-fluoro-4-((3-morpholinoazetidin-1-yl)methyl)benzyl)amino)isoindoline-1,3-dione or a pharmaceutically acceptable salt thereof, wherein:
the compound is administered in a combination therapy with a T cell therapy comprising a dose of engineered cells comprising T cells expressing a chimeric antigen receptor (CAR) that binds cluster of differentiation 19 (CD19), wherein the T cell therapy is administered on Day 1 of the combination therapy; and
the compound is administered as a plurality of intermittent doses administered once every seven days (Q7D) and is administered on Days 8, 15, 22, 29, 36, 43, 50, 57, 64, 71, 78, and 85, wherein each dose is 0.6 mg.

42. The method of any one of claims 1-41, wherein the compound is or comprises a pharmaceutically acceptable salt of (S)-2-(2,6-dioxopiperidin-3-yl)-4-((2-fluoro-4-((3-morpholinoazetidin-1-yl)methyl)benzyl)amino)isoindoline-1,3-dione.

43. The method of any one of claims 1-41, wherein the compound is or comprises (S)-2-(2,6-dioxopiperidin-3-yl)-4-((2-fluoro-4-((3-morpholinoazetidin-1-yl)methyl)benzyl)amino)isoindoline-1,3-dione.

44. The method of any one of claims 1-43, wherein at the time of the initiation of the administration of the compound, the subject does not exhibit a severe toxicity following the administration of the T cell therapy.

45. The method of claim 44, wherein:
the severe toxicity is severe cytokine release syndrome (CRS), optionally grade 3 or higher CRS, prolonged grade 3 or higher CRS, grade 4 CRS, or grade 5 CRS; and/or
the severe toxicity is severe neurotoxicity, optionally grade 3 or higher neurotoxicity, prolonged grade 3 or higher neurotoxicity, grade 4 neurotoxicity, or grade 5 neurotoxicity.

46. The method of any one of claims 1-45, wherein the administration of the compound is suspended and/or the dose of the compound is modified, optionally lowered, if the subject exhibits a toxicity following the administration of the compound, optionally a hematologic toxicity.

47. The method of claim 46, wherein the toxicity is severe thrombocytopenia, optionally grade 4 thrombocytopenia or prolonged grade 4 thrombocytopenia.

48. The method of claim 46, wherein the toxicity is severe neutropenia, optionally grade 4 neutropenia, prolonged grade 4 neutropenia, or febrile neutropenia, optionally grade 3 or higher febrile neutropenia or prolonged grade 3 or higher febrile neutropenia.

49. The method of any one of claims 46-48, wherein the administration of the compound is restarted after the subject no longer exhibits the toxicity.

50. The method of any one of claims 1-49, wherein the cancer is a B-cell malignancy.

51. The method of any one of claims 1-50, wherein the cancer is a non-Hodgkin lymphoma (NHL), optionally wherein the NHL comprises aggressive NHL; diffuse large B cell lymphoma (DLBCL); DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally follicular lymphoma Grade 3B (FL3B); and/or high-grade B-cell lymphoma with MYC and BCL2 and/or BCL6 rearrangements with DLBCL histology (double/triple hit).

52. The method of any one of claims 1-51, wherein the CD19 is a human CD19.

53. The method of any one of claims 1-52, wherein the chimeric antigen receptor (CAR) comprises an extracellular antigen-recognition domain that specifically binds to the CD19 and an intracellular signaling domain comprising an ITAM.

54. The method of claim 53, wherein the intracellular signaling domain comprises a signaling domain of a CD3-zeta (CD3ζ) chain, optionally a human CD3-zeta chain.

55. The method of claim 53 or claim 54, wherein the chimeric antigen receptor (CAR) further comprises a costimulatory signaling region.

56. The method of claim 55, wherein the costimulatory signaling region comprises a signaling domain of CD28 or 4-1BB, optionally human CD28 or human 4-1BB.

57. The method of claim 55 or claim 56, wherein the costimulatory signaling region comprises a signaling domain of human 4-1BB.

58. The method of any one of claims 1-57, wherein the CAR comprises an scFv specific for the CD19; a transmembrane domain; a cytoplasmic signaling domain derived from a costimulatory molecule, which optionally is or comprises a 4-1BB, optionally human 4-1BB; and a cytoplasmic signaling domain derived from a primary signaling ITAM-containing molecule, which optionally is or comprises a CD3zeta signaling domain, optionally a human CD3zeta signaling domain; and optionally wherein the CAR further comprises a spacer between the transmembrane domain and the scFv.

59. The method of any one of claims 1-57, wherein the CAR comprises, in order, an scFv specific for the CD19; a transmembrane domain; a cytoplasmic signaling domain derived from a costimulatory molecule, which optionally is or comprises a 4-1BB signaling domain, optionally a human 4-1BB signaling domain; and a cytoplasmic signaling domain derived from a primary signaling ITAM-containing molecule, which optionally is a CD3zeta signaling domain, optionally human CD3zeta signaling domain; and optionally wherein the CAR further comprises a spacer between the transmembrane domain and the scFv.

60. The method of any one of claims 1-57, wherein the CAR comprises, in order, an scFv specific for the CD19; a spacer; a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.

61. The method of any one of claims 58-60, wherein the spacer is a polypeptide spacer that comprises or consists of all or a portion of an immunoglobulin hinge or a modified version thereof or comprises about 15 amino acids or less.

62. The method of any one of claims 58-61, wherein the spacer comprises or consists of all or a portion of an immunoglobulin hinge, optionally an IgG4 hinge, or a modified version thereof and/or comprises about 15 amino acids or less.

63. The method of any one of claims 58-62, wherein the spacer is at or about 12 amino acids in length and/or comprises or consists of all or a portion of an immunoglobulin hinge, optionally an IgG4, or a modified version thereof

64. The method of any one of claims 58-63, wherein the spacer has or consists of the sequence of SEQ ID NO: 1; a sequence encoded by SEQ ID NO: 2, SEQ ID NO: 30, SEQ ID NO: 31, SEQ ID NO: 32, SEQ ID NO: 33, SEQ ID NO: 34; or a variant of any of the foregoing having at least 85%, 86%, 87%, 88%, 89%, 90%, 91%, 92%, 93%, 94%, 95%, 96%, 97%, 98%, 99% or more sequence identity thereto.

65. The method of any one of claims 58-64, wherein the cytoplasmic signaling domain derived from a costimulatory molecule comprises the sequence set forth in SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto.

66. The method of any one of claims 58-65, wherein the cytoplasmic signaling domain derived from a primary signaling ITAM-containing molecule comprises the sequence of any of SEQ ID NO: 13-15 or a variant thereof having at least 85%, 86%, 87%, 88%, 89%, 90%, 91%, 92%, 93%, 94%, 95%, 96%, 97%, 98%, 99% or more sequence identity thereto.

67. The method of any one of claims 58-66, wherein the scFv comprises:
a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37); and/or
a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40).

68. The method of any one of claims 58-67, wherein the scFv comprises:
a variable heavy chain region of FMC63 and a variable light chain region of FMC63; and/or
a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63; and
optionally wherein the scFv comprises a VH comprising the amino acid sequence set forth in SEQ ID NO: 41, and a VL comprising the amino acid sequence set forth in SEQ ID NO: 42.

69. The method of any one of claims 58-68, wherein the scFv has the sequence of amino acids set forth in SEQ ID NO: 43.

70. The method of any one of claims 1-69, wherein the dose of engineered cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, or 5×10⁷ to 1×10⁸ total CAR-expressing T cells, each inclusive.

71. The method of any one of claims 1-70, wherein the dose of engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

72. The method of any one of claims 1-71, wherein the dose of engineered cells comprises at or about 1×10⁸ CAR-expressing T cells.

73. The method of any one of claims 1-72, wherein the dose of engineered cells is administered parenterally, optionally intravenously.

74. The method of any one of claims 1-73, wherein the T cells are primary T cells obtained from the subject.

75. The method of any one of claims 1-74, wherein the T cells are autologous to the subject.

76. The method of any one of claims 1-73, wherein the T cells are allogeneic to the subject.

77. The method of any one of claims 1-76, wherein the dose of engineered cells comprises CD4+ T cells expressing the CAR and CD8+ T cells expressing the CAR and the administration of the dose comprises administering a plurality of separate compositions, said plurality of separate compositions comprising a first composition comprising one of the CD4+ T cells and the CD8+ T cells and a second composition comprising the other of the CD4+ T cells and the CD8+ T cells.

78. The method of claim 77, wherein:
the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or
the initiation of administration of the first composition and the initiation of administration of the second composition are carried out between about 1 minute and about 1 hour apart or between about 5 minutes and about 30 minutes apart.

79. The method of claim 77 or claim 78, wherein the first composition and second composition are administered no more than 2 hours, no more than 1 hour, no more than 30 minutes, no more than 15 minutes, no more than 10 minutes or no more than 5 minutes apart.

80. The method of any one of claims 77-79, wherein the first composition comprises the CD4+ T cells.

81. The method of any one of claims 77-79, wherein the first composition comprises the CD8+ T cells.

82. The method of any one of claims 77-81, wherein the first composition is administered prior to the second composition.

83. The method of any one of claims 1-82, wherein, prior to the administration of the T cell therapy, the subject has been preconditioned with a lymphodepleting therapy comprising the administration of fludarabine and/or cyclophosphamide.

84. The method of any one of claims 1-82, further comprising, immediately prior to the administration of the T cell therapy, administering a lymphodepleting therapy to the subject comprising the administration of fludarabine and/or cyclophosphamide.

85. The method of claim 83 or claim 84, wherein the lymphodepleting therapy comprises administration of cyclophosphamide at about 200-400 mg/m², inclusive, optionally at or about 300 mg/m², and/or fludarabine at about 20-40 mg/m², optionally 30 mg/m², daily for 2-4 days, optionally for 3 days, or wherein the lymphodepleting therapy comprises administration of cyclophosphamide at about 500 mg/m².

86. The method of any one of claims 83-85, wherein:
the lymphodepleting therapy comprises administration of cyclophosphamide at or about 300 mg/m² and fludarabine at about 30 mg/m² daily for 3 days; and/or
the lymphodepleting therapy comprises administration of cyclophosphamide at or about 500 mg/m² and fludarabine at about 30 mg/m² daily for 3 days.

87. The method of any one of claims 1-86, wherein the subject is a human.

88. The method of any one of claims 1-87, wherein:
at least 35%, at least 40% or at least 50% of subjects treated according to the method achieve a complete response (CR) that is durable, or is durable in at least 60, 70, 80, 90, or 95% of subjects achieving the CR, for at or greater than 6 months or at or greater than 9 months; and/or
at least 60, 70, 80, 90, or 95% of subjects achieving a CR by six months remain in response, remain in CR, and/or survive or survive without progression, for at or greater than 3 months and/or at or greater than 6 months and/or at or greater than nine months; and/or
at least 50%, at least 60% or at least 70% of the subjects treated according to the method achieve objective response (OR), optionally wherein the OR is durable, or is durable in at least 60, 70, 80, 90, or 95% of subjects achieving the OR, for at or greater than 6 months or at or greater than 9 months; and/or
at least 60, 70, 80, 90, or 95% of subjects achieving an OR by six months remain in response or surviving for at or greater than 3 months and/or at or greater than 6 months.

89. The method of any one of claims 1-88, wherein at or immediately prior to the time of the administration of the dose of engineered cells, the subject has relapsed following remission after treatment with, or become refractory to, one or more prior therapies for the lymphoma, optionally one, two, or three prior therapies other than another dose of engineered cells expressing the CAR.

90. The method of any one of claim 1-89, wherein at or prior to the administration of the dose of engineered cells:
the subject is or has been identified as having a double/triple hit lymphoma;
the subject is or has been identified as having a chemorefractory lymphoma, optionally a chemorefractory DLBCL; and/or
the subject has not achieved complete remission (CR) in response to a prior therapy.

91. The method of any one of claims 1-90, wherein the administration of the compound:
reverses an exhaustion phenotype in CAR-expressing T cells in the subject;
prevents, inhibits, or delays the onset of an exhaustion phenotype in CAR-expressing T cells in the subject;
reduces the level or degree of an exhaustion phenotype in CAR-expressing T cells in the subject; or
reduces the percentage or the total number of CAR-expressing T cells in the subject that have an exhaustion phenotype.

92. The method of any one of claims 1-91, wherein the initiation of the administration of the compound is carried out subsequently to the administration of the T cell therapy and, following administration of the compound or initiation thereof, the subject exhibits a restoration or rescue of an antigen- or tumor-specific activity or function of the CAR-expressing T cells in said subject, optionally wherein said restoration, rescue, and/or initiation of administration of said compound is at a point in time after CAR-expressing T cells in the subject or in the blood of the subject have exhibited an exhausted phenotype.

93. The method of any one of claims 1-92, wherein the administration of the compound comprises administration at an amount, frequency and/or duration effective to:
(a) effect an increase in antigen-specific or antigen receptor-driven activity of naïve or non-exhausted T cells in the subject, which optionally comprise T cells expressing said CAR, following exposure of the T cells to CD19 antigen or to an antigen receptor-specific agent, as compared to the absence of said administration of said compound; or
(b) prevent, inhibit or delay the onset of an exhaustion phenotype in naïve or non-exhausted T cells in the subject, which optionally comprise T cells expressing said CAR, following exposure of the T cells to CD19 antigen or to an antigen receptor-specific agent, as compared to the absence of said administration of said compound; or
(c) reverse an exhaustion phenotype in exhausted T cells, optionally comprising T cells expressing said CAR, in the subject, as compared to the absence of said administration of said compound.

94. The method of claim 93, wherein the administration of the compound comprises administration at an amount, frequency and/or duration effective (i) to effect said increase in activity and (ii) to prevent, inhibit or delay said onset of said exhaustion phenotype and/or reverse said exhaustion phenotype.

95. The method of claim 93 or claim 94, wherein the T cells in the subject comprise T cells expressing said CAR and/or the exposure is to CD19 antigen.

96. The method of any one of claims 91-95, wherein the exhaustion phenotype, with reference to a T cell or population of T cells, comprises:
an increase in the level or degree of surface expression on the T cell or T cells, or in the percentage of said population of T cells exhibiting surface expression, of one or more exhaustion markers, optionally 2, 3, 4, 5 or 6 exhaustion markers, compared to a reference T cell population under the same conditions; or
a decrease in the level or degree of an activity exhibited by said T cells or population of T cells upon exposure to a CD19 antigen or antigen receptor-specific agent, compared to a reference T cell population under the same conditions.

97. The method of claim 96, wherein the increase in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

98. The method of claim 96, wherein the decrease in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

99. The method of any one of claims 96-98, wherein the reference T cell population is a population of T cells known to have a non-exhausted phenotype, is a population of naïve T cells, is a population of central memory T cells, or is a population of stem central memory T cells, optionally from the same subject, or of the same species as the subject, from which the T cell or T cells having the exhaustion phenotype are derived.

100. The method of any one of claims 96-99, wherein the reference T cell population (a) is a subject-matched population comprising bulk T cells isolated from the blood of the subject from which the T cell or T cells having the exhaustion phenotype is derived, optionally wherein the bulk T cells do not express the CAR and/or (b) is obtained from the subject from which the T cell or T cells having the exhaustion phenotype is derived, prior to receiving administration of a dose of T cells expressing the CAR.

101. The method of any one of claims 96-100, wherein the reference T cell population is a composition comprising a sample of T cells expressing the CAR, or pharmaceutical composition comprising T cells expressing the CAR, prior to its administration to the subject, optionally wherein the composition is a cryopreserved sample.

102. The method of any one of claims 96-101, wherein the one or more exhaustion markers is an inhibitory receptor.

103. The method of any one of claims 96-102, wherein the one or more exhaustion markers is selected from among PD-1, CTLA-4, TIM-3, LAG-3, BTLA, 2B4, CD160, CD39, VISTA, and TIGIT.

104. The method of any one of claims 96-103, wherein the activity is one or more of proliferation, cytotoxicity or production of one or a combination of inflammatory cytokines, optionally wherein the one or a combination of cytokines is selected from the group consisting of IL-2, IFN-gamma and TNF-alpha.

105. The method of any one of claims 96-104, wherein the exposure to said CD19 antigen or antigen receptor-specific agent comprises incubation with the CD19 antigen or antigen receptor-specific agent, optionally an agent that binds the antigen-binding domain of the CAR.

106. The method of claim 105, wherein the exposure to the CD19 antigen or antigen receptor-specific agent comprises exposing the T cells to CD19 antigen-expressing target cells, optionally cells of the B cell malignancy.


SEQUENCE LISTING (59 sequences)

SEQ ID NO: 1 - protein, 12 aa, Homo sapiens
  spacer (IgG4hinge)
ESKYGPPCPPCP

SEQ ID NO: 2 - DNA, 36 bp, Homo sapiens
  spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28 (amino acids 153-179 of Accession No. P10747)
FWVLVVVGGVLACYSLLVTVAFIIFWV

SEQ ID NO: 9 - protein, 66 aa, Homo sapiens
  CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28 (amino acids 180-220 of P10747)
RSKRSRLLHSDYMNMTPRRPGPTRKHYQPYAPPRDFAAYRS

SEQ ID NO: 11 - protein, 41 aa, Homo sapiens
  CD28 (LL to GG)
RSKRSRGGHSDYMNMTPRRPGPTRKHYQPYAPPRDFAAYRS

SEQ ID NO: 12 - protein, 42 aa, Homo sapiens
  4-1BB (amino acids 214-255 of Q07011.1)
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 10 aa, Artificial Sequence
  Linker
  REPEAT (5)...(9): SGGGG is repeated 5 times
PGGGSGGGGP

SEQ ID NO: 23 - protein, 17 aa, Artificial Sequence
  Linker
GSADDAKKDAAKKDGKS

SEQ ID NO: 24 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 25 - protein, 22 aa, Artificial Sequence
  GMCSFR alpha chain signal sequence
MLLLVTSLLLCELPHPAFLLIP

SEQ ID NO: 26 - protein, 18 aa, Artificial Sequence
  CD8 alpha signal peptide
MALPVTALLLPLALLLHA

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 12 aa, Artificial Sequence
  Hinge
ESKYGPPCPPCP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  CDR L1
RASQDISKYLN

SEQ ID NO: 36 - protein, 7 aa, Artificial Sequence
  CDR L2
SRLHSGV

SEQ ID NO: 37 - protein, 9 aa, Artificial Sequence
  CDR L3
GNTLPYTFG

SEQ ID NO: 38 - protein, 5 aa, Artificial Sequence
  CDR H1
DYGVS

SEQ ID NO: 39 - protein, 16 aa, Artificial Sequence
  CDR H2
VIWGSETTYYNSALKS

SEQ ID NO: 40 - protein, 7 aa, Artificial Sequence
  CDR H3
YAMDYWG

SEQ ID NO: 41 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 5 aa, Artificial Sequence
  CDR H1
SYWMN

SEQ ID NO: 48 - protein, 17 aa, Artificial Sequence
  CDR H2
QIYPGDGDTNYNGKFKG

SEQ ID NO: 49 - protein, 13 aa, Artificial Sequence
  CDR H3
KTISSVVDFYFDY

SEQ ID NO: 50 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, sArtificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  HC-CDR3
HYYYGGSYAMDY

SEQ ID NO: 55 - protein, 7 aa, Artificial Sequence
  LC-CDR2
HTSRLHS

SEQ ID NO: 56 - protein, 9 aa, Artificial Sequence
  LC-CDR3
QQGNTLPYT

SEQ ID NO: 57 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 58 - protein, 5 aa, Artificial Sequence
  Hinge
  VARIANT (1)...(1): Xaa is glycine, cysteine or arginine
  VARIANT (4)...(4): Xaa is cysteine or threonine
XPPXP

SEQ ID NO: 59 - protein, 18 aa, Artificial Sequence
  Linker
GSTSGSGKPGSGEGSTKG